(19) United States
(12) Patent Application Publication
(10) Pub. No.: US 2011/0061116 A1
(43) Pub. Date: Mar. 10, 2011

(54) Animal Model of Synovial Sarcoma

(75) Inventors: Malay Haldar, Salt Lake City, UT (US); Mario R. Capecchi, Salt Lake City, UT (US)

(73) Assignee: UNIVERSITY OF UTAH RESEARCH FOUNDATION

(21) Appl. No.: 12/666,268

(22) PCT Filed: Jul. 22, 2008
(86) PCT No.: PCT/US08/70769

§ 371 Date: May 17, 2010

Related U.S. Application Data
(60) Provisional application No. 60/952,979, filed on Jul. 31, 2007.

(51) Int. Cl. A01K 67/00; C12N 5/10

(52) U.S. Cl. 800/10; 435/354

(57) ABSTRACT

Synovial sarcoma is an aggressive soft-tissue malignancy. Disclosed herein is an animal model of synovial sarcoma wherein one or more myogenic cells of the animal express recombinant SYT-SSX fusion polypeptide. Using this model, myoblasts were identified as a source of synovial sarcoma. Remarkably, within the skeletal muscle lineage, while expression of the oncoprotein in immature myoblasts leads to induction of synovial sarcoma with 100% penetrance, its expression in more differentiated cells induces myopathy without tumor induction. In addition, early widespread expression of the disclosed fusion protein disrupts normal embryogenesis, causing lethality.

### CROSS-REFERENCE TO RELATED APPLICATIONS

This application claims benefit of U.S. Provisional Application No. 60/952,979, filed Jul. 31, 2007, which is hereby incorporated herein by reference in its entirety.

### STATEMENT REGARDING FEDERALLY SPONSORED RESEARCH

This invention was made with government support under Grant RO1 GM21168-33 awarded by the National Institutes of Health. The government has certain rights in the invention.

### BACKGROUND

Synovial sarcoma accounts for 7%-10% of all soft-tissue sarcomas, frequently affecting adolescents and young adults. Metastasis is common and usually targeted to lungs, lymph nodes, and bone marrow (Weiss and Goldblum, 2001). The name “synovial sarcoma” was initially coined for tumors arising near joints and having some microscopic resemblance to synovial tissue. However, this tumor can arise, although rarely, in sites away from joints such as head and neck, pharynx, lungs, and heart, which contradicts this nomenclature. Several studies have demonstrated a lack of synovial differentiation in synovial sarcoma tumor cells and showed that these tumors express markers of both epithelial and mesenchymal differentiation, although they do not resemble any specific tissue type (Fisher, 1986; Smith et al., 1995). Synovial sarcoma is now regarded as a neoplasm of “uncertain differentiation.” Based on histopathology, synovial sarcomas are divided into biphasic, monophasic, and poorly differentiated subtypes. The presence of epithelioid cells often arranged in whorls or primitive gland-like structures along with the presence of spindle-shaped cells is a hallmark of the biphasic subtype, while the monophasic subtype is marked by a predominance of spindle cells. The poorly differentiated subtype comprises primitive small round cells similar to Ewing's sarcoma. Immunohistochemistry plays an important role in diagnosis, the hallmark being expression of both epithelial markers (cytokeratins) and mesenchymal markers (vimentin). Bcl-2 overexpression is also frequently observed in these tumors (Hibshoosh and Lattes, 1997; Pelmus et al., 2002).

Current methods to investigate synovial sarcoma and evaluate new therapies generally involve in vitro cell culture methods and xenograft modles that involve transplanting transformed cancer cells of another species such as humans into mouse that lacks functional immune system (e.g., nude mice). These immunodeficient mice then develop tumors since the lack of immunity prevents these mice from rejecting the transplanted cells. However, tumors developed in such a way do not recapitulate the human case, especially since the immune system that plays a very vital role in tumor induction, progression and drug response is not functional in these mice. Thus, needed in the art are improved animal models of synovial sarcoma to identify and evaluate new therapies.

### BRIEF SUMMARY

In accordance with the purpose of this invention, as embodied and broadly described herein, this invention relates to animal models of synovial sarcoma and methods of making and using same.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows generation of targeted mouse lines. As shows in FIG. 1A, the SSM2 targeting vector comprises: LoxP flanked Neomycin resistance gene (Neo) with 3′ polyadenylation signal (PolyA) and a 5′ Pgk promoter. This was followed by SYT-SSX2 cDNA and an encephalomyocarditis virus internal ribosomal entry site (IRES) fused to enhanced green fluorescent protein (EGFP) cDNA. This entire construct was flanked by ROSA26 homology region. A negative selection cassette comprising of a Pgk promoter driving expression of diphtheria toxin A (DTA) was placed after the ROSA homology region. SSM1 targeting vector had the same components except IRES-EGFP. Presence of CRE led to recombination between the two LoxP sites thereby removing the Neomycin gene along with its polyA allowing transcription of SYT-SSX2 and EGFP from the endogenous ROSA promoter. As shown in FIG. 1B, the Myf5 targeting vector contained an encephalomyocarditis virus internal ribosomal entry site (IRES) fused to the CRE recombinase cDNA (CRE). This was followed by neomycin resistance gene (Neo) expressed by MC1 promoter and flanked by two FRT sequence to allow removal of MC1-Neo by breeding to flippase expressing mouse if desired. This entire construct was flanked by sequences homologus region to he Myf5 3′UTR. A thymidine kinase 1 (TK1) negative selection cassette was placed after Myf5 homology region. This strategy enables transcription of a bicistronic Myf5-IRES-CRE RNA leading to expression of CRE recombinase without interfering with Myf5 expression.

FIG. 2 shows tumor induction within Myf5 lineage. FIG. 2A shows Myf5-lineage in somites of E 9.5 Myf5-CRE/ROSA-YFP embryos based on whole-mount fluorescence (Aa). FIG. 2Ab shows a wild type littermate embryo. Adult Myf5-CRE/ROSA-YFP skeletal muscle sections demonstrated YFP expression (Ac) within skeletal muscle fibers expressing skeletal muscle specific myosin (Ad). Nuclei were stained by DAPI. FIG. 2B shows tumors within skeletal musculature of limbs (Ba and Bd) and intercostal region (Bb, arrows). Tumors were fluorescent due to EGFP expression (Bc, arrows and Be). Figure Bf shows a fluorescent potential metastatic lesion in brain.

FIG. 3 shows tumor histology and immunohistochemistry. FIG. 3A shows by biphasic histology (Aa and Ab) epithelioid components arranged in glandular pattern (Ab, arrow). Also shown is a biphasic tumor with a cystic space (Ac, arrow) and hemorrhage (Ac, arrow). Also shown is a monophasic tumor composed of spindle cells (Ad) showing trapped skeletal muscle fibers (Ad, arrow). Tumors show myxoid change detected by alcian blue staining (Ae) and fibrosis detected by Masson's trichrome staining (Af, arrow). As shown in FIG. 3B, tumors were positive for vimentin (Ba), CAM 5.2 (Bb), Cytokeratin AE1/AE2 (Bc) and Bcl-2 (Bd). Tumors expressed proliferation marker Mib (Be) and were usually negative for myogenin (Bf).

FIG. 4 shows microarray analysis of mouse tumors. As shown in FIG. 4A, hierarchical clustering shows expected segregation of tumors and normal skeletal muscle. FIG. 4B shows strategy for gene set enrichment analyses. The initial GSEA is shown in FIG. 4Ba. The SYT-SSX model synovial subset derived from this initial GSEA is represented in FIG. 4Bb. The human synovial sarcoma control gene set is represented by FIG. 4Bc. All three gene sets were compared in sequence to the phenotypes present in the remaining human tumor data sets (FIG. 4Bd).

FIG. 5 shows SYT Expression within Myf5 Lineage and Myf5 Lineage Restriction by SYT-SSX2. As shown in FIG. 5A, SYT expression (nuclear) was detected within YFP (cytoplasmic) expressing Myf5-lineage in Myf5-CRE/ROSA-YFP E 15.5 embryos (left panel) and adult skeletal muscle (right panel). FIG. 5B shows a tumor arising near ribs (FIG. 5Ba and Bb) where the all tumor cells were expressing EGFP (cytoplasmic) while surrounding and trapped muscle fibers expressing myosin (cytoplasmic staining in myofibers) were negative for EGFP. Nuclei were stained by DAPI. FIG. 5C shows Myf5-lineage in Myf5-CRE/ROSA-YFP E11.5 embryos (left panel) was marginally reduced in E11.5 Myf5-CRE/SSM2 embryo (middle panel) based on whole-mount fluorescence. Insets show dermomyotomal regions at a higher magnification. Right panel shows wild type littermate embryo. FIG. 5D shows Myf5-lineage was robust within Myf5-CRE/ROSA-YFP E15.5 embryos (FIG. 5Da) but undetectable in E15.5 Myf5-CRE/SSM2 embryos (FIG. 5Db). Figure Dc shows wild type littermate embryo. This is also apparent by comparing sections through Myf5-CRE/ROSA-YFP E15.5 embryos (FIG. 5Dd) to Myf5-CRE/SSM2 E15.5 embryos (FIG. 5De). In Dd, lineage marker YFP (cytoplasmic) co-localized with skeletal muscle specific MyoD (nuclear) compared to near absence of the lineage marker EGFP (cytoplasmic) in the presence of normal MyoD expression (nuclear) in De. FIG. 5E shows significant apoptosis (nuclear staining) within somitic lineage of Myf5 expressing EGFP (cytoplasmic) in E11.5 Myf5-CRE/SSM2 embryos (left panel). This is in contrast to absence of apoptosis (no stained nuclei) within EGFP expressing Myf-5 lineage (cytoplasmic) near mesenchymal condensations of future ribs (Right panel, arrow) of the same embryo.

FIG. 6 shows SYT-SSX2 disrupts normal development. FIG. 6A shows disorganized Hprt-Cre/SSM2 fetal tissue (right panel) expressing EGFP (left panel). FIG. 6B shows Pax3 lineage within E10.5 Pax3-Cre-KI/SSM2 embryo was detectable (left panel). FIG. 6B, right panel shows wild-type (WT) littermate embryo. FIG. 6C, left panel shows Pax7 lineage within Pax7-Cre/ROSA-YFP E15.5 embryos, while the middle panel shows significantly reduced Pax7 lineage within Pax7-Cre/SSM2 embryo based on whole-mount fluorescence. The maxillary and nasal regions of Pax7-Cre/SSM2 embryo showed a larger proportion of surviving Pax7 lineage compared to other regions (white arrows). Right panel shows wild-type (WT) littermate embryos. As shown in FIG. 6D, myopathic skeletal muscle of Myf6-CRE/SSM adult mice showed abnormal wavy fibers, myonuclear chain (FIG. 6Dc, arrow) and significant variations in cross-sectional diameter between fibers (FIG. 6Dd) with occasional vaccuolation (FIG. 6Dd, arrow) and central nuclei (FIG. 6Dd, arrow). FIGS. 6Da and 6Db show skeletal muscle sections from wild type (WT) littermate. The myopathic skeletal muscle expresses EGFP detected in whole mount (FIG. 6De) and sections (FIG. 6Df). Apoptosis within myopathic muscles was detected by TUNEL assay (FIG. 6Dh, nuclear stainingi). Figure Dg shows TUNEL-negative control skeletal muscle section from wild type (WT) littermate. FIGS. 6Di and 6Dj correspond to the same field in FIGS. 6Dg and 6Dh and show nuclei stained by DAPI.

FIG. 7 shows results of expressing SYT-SSX2 fusion protein within various cells of skeletal muscle lineage. The box highlights the suspected cell of origin: myoblasts arising from postnatal satellite cells.

FIG. 8A shows Southern blot strategy for SSM1 and SSM2 mice. Shown is a schematic of the ROSA locus targeted with SSM2 with relevant restriction enzyme sites used for Southern blotting. Except for the absence of IRES-EGFP, SSM1 has the same architecture. DNA extracted from R1 embryonic stem (R1ES) cells targeted with the SSM2 or SSM1 constructs were digested with EcoRV restriction enzyme, run on a 0.8% agarose gel, transferred to a supported nitrocellulose membrane (Optitran—BA-S 85 from Schleischer & Scheuell), and hybridized with a radioactive probe to the 5′ end of the ROSA locus outside the targeted region. The radioactive probe, generated by random priming, detected a 4.1 Kb targeted band and an 11.5 Kb wild type band (FIG. 8Aa). An internal probe to the neomycin resistance gene was also designed and used against AvrII restriction enzyme digested DNA from the targeted R1ES cells that detected a 10.4 Kb targeted band for SSM1 and a 7 Kb targeted band for SSM2 (FIG. 8Ab). A radioactive probe against the EGFP coding region was also designed that detected an 11.6 Kb band on EcoRV digested DNA from R1 cell targeted with SSM2 (FIG. 8Ac). A 3′ external probe, outside the targeted area, was also designed that detected a 9.2 Kb targeted band and 11.5 Kb wild type band on EcoRV digested DNA from SSM1 targeted R1 cells (Ad). FIG. 8B shows Southern blot strategy for Myf5-Cre mice: DNA extracted from targeted R1ES cells were digested with EcoRV restriction enzyme, run on a 0.8% agarose gel, transferred to a supported nitrocellulose membrane (Optitran—A-S 85 from Schleischer & Scheuell), and hybridized with a radioactive probe to the 3′ UTR outside the targeted region. The radioactive probe, generated by random priming, detected a targeted band of 11.7 Kb that is a 3.9 Kb downshift from the wild type band of 15.6 Kb (FIG. 8Ba). Another probe against Cre coding region was designed and used against AvrII restriction enzyme digested DNA from the targeted cells to detect a 4 Kb band from the targeted locus.

FIG. 9A shows phenotype of Myf5-Cre/ROSA-DTA mice. There was no difference in size between 10-week-old siblings of Myf5-Cre/ROSA-DTA and Control (WT) mice harboring only the uninduced allele of DTA (FIG. 9Aa). About 8%-10% of Myf5-Cre/DTA mice were significantly smaller than their siblings (FIG. 9Ab). These small Myf5-Cre/ROSA-DTA mice showed mild skeletal muscle anomalies (signs of regeneration) but otherwise normal skeletal muscle fibers apparent in cross sections (low magnification [FIG. 9Ac] and high magnification [FIG. 9Ad]) as well as longitudinal sections (low magnification [FIG. 9Ae] and high magnification [FIG. 9Af]). The small-sized Myf5-Cre/ROSA-DTA mice (similar to the small born Myf5-Cre/SSM mice) had smaller size at birth and perform poorly right from P0 compared to control siblings. This is unlike the Myf6-Cre/SSM myopathic mice that appeared normal at birth but gradually develop weakness and reduced vitality with age eventually leading to their death. FIG. 9B shows RTPCR on total RNA (treated with DNAse) from a tumor demonstrates the presence of SYT-SSX2 (FIG. 9B, left panel). A control PCR with the same set of primers without reverse transcription demonstrated the absence of any contaminating DNA in extracted RNA from tumor sample.

FIG. 10 shows SYT-SSX model synovial subset extraction. The genes in the murine tumor gene set to the left of the enrichment peak in the Detwiller et al. synovial sarcoma versus others comparison were designated as the SYT-SSX model synovial signature.

FIG. 11 shows CreER strategy for inducible conditional expression of the Synovial Sarcoma-Associated SYT-SSX2 fusion Oncogene. Triangle denotes heat-shock proteins interacting with the CreER thereby sequestering CreER in the cytoplasm and preventing its entry into the nucleus. Application of tamoxifen (“T”) leads to nuclear translocation of CreER. In the nucleus, CreER mediates genetic recombination between the two LoxP sequences and removes the transcriptional stop signal (STOP) allowing transcription of the SYT-SSX2 and EGFP bicistronic messenger RNA. This bicistronic RNA is translated into two individual proteins; SYT-SSX2 and EGFP in the cytoplasm.

FIG. 12 shows random sporadic expression of SYT-SSX2 in multiple tissue generates tumors in mice. The conditional SSM mouse line (A) was bred to the Rosa-CreER mouse line (B), which expresses the CreER fusion protein ubiquitously in all tissue/cell type. The CreER expression was driven by the mouse endogenous Rosa Promoter that is known to be ubiquitously active in all mouse tissue. In the presence of exogenously applied tamoxifen (intraperitoneal injection) or without it (“leaky” nuclear translocation of CreER) CreER mediated removal of the transcriptional stop signal leads to expression of SYT-SSX2 and EGFP in multiple tissue types within the progeny SSM/Rosa-CreER mice. Tumors were generated (C) expressing the enhanced green fluoroscent (EGFP) marker protein that was grossly visible under a fluoroscent scope (D) as well as in micrograph (E). The tumor histology was strikingly similar to human Synovial Sarcomas.

FIG. 13 shows mouse tumors mimic Synovial Sarcomas. Expression of the mesenchymal marker vimentin (FIG. 13Aa), epithelial marker cytokeratin (FIG. 13Ab) and the anti-apoptotic protein Bcl-2 (FIG. 13Ac) in the mouse tumors indicate a diagnosis of synovial sarcoma. FIG. 13B shows microarray comparison of mouse synovial sarcoma induced by SYT-SSX2 in myoblasts or randomly in multiple cell types with that of various mouse tumors.

### DETAILED DESCRIPTION

The disclosed method and compositions may be understood more readily by reference to the following detailed description of particular embodiments and the Example included therein and to the Figures and their previous and following description.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a peptide is disclosed and discussed and a number of modifications that can be made to a number of molecules including the peptide are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, is this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

Those skilled in the art will recognize, or be able to ascertain using no more than routine experimentation, many equivalents to the specific embodiments of the method and compositions described herein. Such equivalents are intended to be encompassed by the following claims.

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

### A. COMPOSITIONS

Provided herein is a non-human animal model of synovial sarcoma, wherein one or more myogenic cells of the animal express recombinant SYT-SSX fusion polypeptide.

1. Synovial Sarcoma

Synovial sarcoma is marked by a signature genetic event, the t(X; 18) translocation-mediated fusion of the SYT gene on chromosome 18q11 to either SSX1, SSX2, or, very rarely, the SSX4 gene located on chromosome Xpll (Clark et al., 1994; Crew et al., 1995; de Leeuw et al., 1995; dos Santos et al., 2001; Limon et al., 1986; Panagopoulos et al., 2001; Skytting et al., 1999; Smith et al., 1987). This translocation is specific to synovial sarcoma. While the presence of SYT-SSX transcript is considered diagnostic for synovial sarcoma, the reciprocal SSX-SYT transcripts are frequently absent within these tumors (Guillou et al., 2001; Hiraga et al., 1998; Ladanyi and Bridge, 2000; Panagopoulos et al., 2001; Poteat et al., 1995; Willeke et al., 1998). Studies have demonstrated that human SYT-SSX1 transforms rat fibroblasts, and the transformed cells formed tumors within nude mice (Nagai et al., 2001). The 50 translocation partner SYT is evolutionarily conserved, possesses promoter architecture of housekeeping genes, and is widely expressed in humans and mice (de Bruijn et al., 1996, 2001). It is a putative transcriptional coactivator and is thought to exert its effect by binding to chromatin remodelers (Perani et al., 2003; Thaete et al., 1999). The 30 translocation partner SSX is a family of closely related genes on the X chromosome and is believed to be transcriptional corepressors. SSX expression in adults is restricted to testes, although it is occasionally expressed in certain tumors as well (Clark et al., 1994; Crew et al., 1995; Gure et al., 1997; Lim et al., 1998). The t(X;18)-generated SYT-SSX fusion protein retains the activation domain of SYT along with the repressor domain of SSX, lacks a DNA binding domain, and probably acts via interaction with chromatin remodelers (dos Santos et al., 2000, 2001; Nagai et al., 2001). Several studies have also shown that the type of translocation has a bearing on the prognosis and histology of synovial sarcoma. The SYT-SSX1 fusion type has been shown to be associated with biphasic histology and a worse prognosis compared to the predominantly monophasic SYT-SSX2 subtype (de Leeuw et al., 1994; Kawai et al., 1998; Ladanyi et al., 2002; Renwick et al., 1995).

However, while the SYT-SSX2 translocation driven chimeric protein was associated with human synovial sarcoma, it was not clear whether presence the translocation product itself was sufficient to induce synovial sarcoma. Multiple genetic hits are frequently required for tumor induction and progression. In addition, the origin of synovial sarcoma was unknown. Further, as disclosed herein, the non-human, e.g., mouse, SSX family of genes was not previously characterized. Thus, it was not clear whether expressing human chimeric SYT-SSX protein would have an equivalent effect (tumor induction) in a non-human animal model.

Disclosed herein is the generation of an animal model of synovial sarcoma expressing the SYT-SSX2 fusion protein within myoblasts of skeletal muscle lineage. Taking into consideration that synovial sarcoma is a somatic genetic disease and tumorigenesis is dependent upon a permissive microenvironment, the herein disclosed animal model can conditionally express human SYT-SSX2 fusion protein in the presence of site-specific transactivator, such as Cre recombinase, thereby allowing the investigation of the transforming role of this protein in chosen tissues at specific times. The tissue or cell of origin for synovial sarcoma is unclear. However, its frequent occurrence within or in proximity to skeletal muscles indicates that transformation of an undifferentiated cell of skeletal muscle lineage gives rise to these tumors. Disclosed herein is the generation of synovial-sarcoma-like tumors in animals expressing SYT-SSX2 within skeletal-muscle-specific Myf5 lineage. The tumors can be generated at 20, 25, 30, 35, 40, 45, 50, 55, 60, 65, 70, 75, 80, 85, 90, 95, 100% penetrance. These tumors can recapitulate the histopathological, immunohistochemical, and transcriptional profile of human synovial sarcoma.

2. SYT-SSX Fusion Protein

Disclosed herein is an animal model comprising a SYT-SSX fusion protein. Fusion proteins, also know as chimeric proteins, are proteins created through the joining of two or more genes which originally coded for separate proteins. Translation of this fusion gene results in a single polypeptide with function properties derived from each of the original proteins. Recombinant fusion proteins can be created artificially by recombinant DNA technology for use in biological research or therapeutics. Chimeric mutant proteins occur naturally when a large-scale mutation, typically a chromosomal translocation, creates a novel coding sequence containing parts of the coding sequences from two different genes.

The functionality of fusion proteins is made possible by the fact that many protein functional domains are modular. In other words, the linear portion of a polypeptide which corresponds to a given domain, such as a tyrosine kinase domain, may be removed from the rest of the protein without destroying its intrinsic enzymatic capability. Thus, any of the herein disclosed functional domains can be used to design a fusion protein.

A recombinant fusion protein is a protein created through genetic engineering of a fusion gene. This typically involves removing the stop codon from a cDNA sequence coding for the first protein, then appending the cDNA sequence of the second protein in frame through ligation or overlap extension PCR. That DNA sequence will then be expressed by a cell as a single protein. The protein can be engineered to include the full sequence of both original proteins, or only a portion of either.

If the two entities are proteins, often linker (or “spacer”) peptides are also added which make it more likely that the proteins fold independently and behave as expected. Especially in the case where the linkers enable protein purification, linkers in protein or peptide fusions are sometimes engineered with cleavage sites for proteases or chemical agents which enable the liberation of the two separate proteins. This technique is often used for identification and purification of proteins, by fusing a GST protein, FLAG peptide, or a hexa-his peptide (aka: a 6xhis-tag) which can be isolated using nickel or cobalt resins (affinity chromatography). Chimeric proteins can also be manufactured with toxins or anti-bodies attached to them in order to study disease development.

Alternatively, internal ribosome entry sites (IRES) elements can be used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (U.S. Pat. Nos. 5,925,565 and 5,935,819; PCT/US99/05781). IRES sequences are known in the art and include those from encephalomycarditis virus (EMCV) (Ghattas, I. R. et al., Mol. Cell. Biol., 11:5848-5849 (1991); BiP protein (Macejak and Sarnow, Nature, 353:91 (1991)); the Antennapedia gene of drosophilia (exons d and e) [Oh et al., Genes & Development, 6:1643-1653 (1992)); those in polio virus [Pelletier and Sonenberg, Nature, 334:320325 (1988); see also Mountford and Smith, TIG, 11:179-184 (1985)).

Thus, the herein disclosed nucleic acids can further comprise a one or more additional nucleic acid sequences encoding one or more proteins, such as a marker, operably linked to the expression control sequence, wherein the nucleic acid sequences are separated by one or more internal ribosome entry sites (IRES). In addition, the expression control sequence of the above nucleic acids, such as the myogenic expression control sequences, can be substituted with full or partial genes, such as a myogenic gene, wherein the promoter for this gene is operably linked to the SYT-SSX transgenes or transactivators using one or more IRES.

For example, the second nucleic acid of the second example can comprises a Myf5 gene, or fragment thereof comprising at least the Myf5 promoter, that is 5′ to an internal ribosome entry site (IRES) that is 5′ to the nucleic acid encoding the transactivator polypeptide.

The IRES element is an internal ribosomal entry sequence (integrated) which can be iosolated from the encephalomyocarditis crius (ECMV). This element allows multiple genes to be expressed and correctly translated when the genes are on the same construct. IRES sequences are discussed in for example, U.S. Pat. No. 4,937,190, which is herein incorporated by reference at least for material related to IRES sequences and their use. The IRES sequence can be obtained from a number of sources including commercial sources, such as the pIRES expressing vector from Clonetech (Clontech, Palo Alto Calif. 94303-4230).

The SYT protein appears to act as a transcriptional coactivator and the SSX proteins as corepressors. Thus, the recombinant SYT-SSX fusion polypeptide of the herein disclosed non-human animal model of synovial sarcoma can comprise a first peptide sequence comprising at least the activation activity of SYT and a second peptide sequence having at least the repressor activity of SSX.

For example, the SYT-SSX fusion polypeptide can have at least about 60%, at least about 65%, at least about 70%, at least about 75%, at least about 80%, at least about 85%, at least about 90%, at least about 95%, at least about 96%, at least about 97%, at least about 98%, at least about 99% or at least about 100% sequence identity to the amino acid sequence SEQ ID NO:2, or a fragment thereof of at least 20, 30, 40, 50, 60, 70, 80, 90, 100, 150, 200, 250, 300, 350, 400, 450, or 500 amino acids in length.

i. SYT

The SYT protein (SS18) was found to contain a conserved 54-amino acid domain at the N terminus of the protein (the SNH domain) that is found in proteins from a wide variety of species, and a C-terminal domain, rich in glutamine, proline, glycine, and tyrosine (the QPGY domain), which contains the transcriptional activator sequences.

Thus, the SYT-SSX fusion polypeptide can comprise a SYT N-terminal (SNH) domain. The SNH domain can have at least about 60%, at least about 65%, at least about 70%, at least about 75%, at least about 80%, at least about 85%, at least about 90%, at least about 95%, at least about 96%, at least about 97%, at least about 98%, at least about 99% or at least about 100% sequence identity to the amino acid sequence SEQ ID NO:7, or a fragment thereof of at least 20, 25, 30, 35, 40, 45, 50, 55, or 60 amino acids in length.

Thus, the first peptide sequence comprising at least the activation domain of SYT can comprise a QPGY domain. The QPGY domain can have at least about 60%, at least about 65%, at least about 70%, at least about 75%, at least about 80%, at least about 85%, at least about 90%, at least about 95%, at least about 96%, at least about 97%, at least about 98%, at least about 99% or at least about 100% sequence identity to the amino acid sequence SEQ ID NO:10, or a fragment thereof of at least 20, 25, 30, 35, 40, 45, 50, 55, 60, 65, 70, 75, 80, 85, 90, 95, 100, 110, 120, 130, 140, 150, 160, 170, or 180 amino acids in length.

Deletion of the SNH domain resulted in a more active transcriptional activator, indicating that this domain acts as an inhibitor of the activation domain. The C-terminal SSX domain present in the SYT-SSX translocation protein contributes a transcriptional repressor domain to the protein. Thus, the fusion protein can have transcriptional activating and repressing domains.

The SYT of the first peptide sequence can be derived from any animal, including a mammal. For example, the animal can be selected from the group consisting of avian, bovine, canine, caprine, equine, feline, leporine, murine, ovine, porcine, primate. Thus, the SYT of the first peptide sequence can be human SYT.

The mouse homolog of SYT was isolated and sequenced in full by de Bruijn et al. (1996), who referred to the gene as syt. It was found to have been conserved during evolution and to be part of a region of synteny between human and mouse chromosomes 18. In early embryogenesis, mouse syt is ubiquitously expressed. In later stages, the expression becomes confined to cartilage tissues, specific neuronal cells, and some epithelium-derived tissues. In mature testis, expression was specifically observed in primary spermatocytes.

The syt gene contains 11 exons spanning approximately 70 kb. The promoter region lacks CAAT and TATA boxes but contains CpG islands, indicating that syt is a housekeeping gene.

ii. SSX

The SSX1 and SSX2 genes encode closely related proteins (81% identity) of 188 amino acids that are rich in charged amino acids. The N-terminal portion of each SSX protein exhibits homology to the Kruppel-associated box (KRAB), a transcriptional repressor domain previously found only in Kruppel-type zinc finger proteins, e.g., zinc finger protein-117 (ZNF117) and ZNF83.

In some aspects, the SYT-SSX fusion polypeptide does not comprise a transcriptional repressor domain, such as, for example, a KRAB domain.

The SYT-SSX fusion polypeptide can comprise an SSX Repression domain (SSXRD domain). The SSXRD domain can have at least about 60%, at least about 65%, at least about 70%, at least about 75%, at least about 80%, at least about 85%, at least about 90%, at least about 95%, at least about 96%, at least about 97%, at least about 98%, at least about 99% or at least about 100% sequence identity to the amino acid sequence SEQ ID NO:15, or a fragment thereof of at least 20, 25, 30, 35, 40, 45, 50, 55, 60, 65, 70, 75, or 80 amino acids in length.

There are at least 5 variants of the SSX gene, SSX1, SSX2, SSX3, SSX4, and SSX5, but only SSX1 and SSX2 had been shown to fuse with the SYT gene in the translocation t(X; 18) in synovial sarcoma. Skytting et al. (1999) analyzed the type of SYT-SSX fusion mRNA in biopsy specimens from 3 synovial sarcomas by nested RT-PCR amplification. Two of the tumors were positive with either the SSX1 or the SSX2 primers, and 1 was positive with both RT-PCR assays. The third specimen showed an 187-bp fragment with 100% homology to the SYT gene linked to a 246-bp fragment with 100% homology to the long splice variant of SSX4. The breakpoint on SSX4 was identical to that observed for SSX1 and SSX2; all of the SSX genes involved in the SYT-SSX fusion genes are split between the fourth and fifth exons. Thus, as disclosed herein, the SSX of the SYT-SSX fusion polypeptide can be SSX1, SSX2, or SSX4.

Not wishing to be bound by theory, the disclosed SYT-SSX fusion polypeptide can act via interaction with chromatin remodelers rather than by binding DNA. Thus, the SYT-SSX fusion polypeptide can lack a DNA binding domain.

The SYT of the first peptide sequence can be derived from any animal, including a mammal. For example, the animal can be selected from the group consisting of avian, bovine, canine, caprine, equine, feline, leporine, murine, ovine, porcine, primate. Thus, the SYT of the first peptide sequence can be human SSX.

3. SYT-SSX Transgenic Animals

By a “transgene” is meant a nucleic acid sequence that is inserted by artifice into a cell and becomes a part of the genome of that cell and its progeny. Such a transgene may be (but is not necessarily) partly or entirely heterologous (e.g., derived from a different species) to the cell. The term “transgene” broadly refers to any nucleic acid that is introduced into an animal's genome, including but not limited to genes or DNA having sequences which are perhaps not normally present in the genome, genes which are present, but not normally transcribed and translated (“expressed”) in a given genome, or any other gene or DNA which one desires to introduce into the genome. This may include genes which may normally be present in the nontransgenic genome but which one desires to have altered in expression, or which one desires to introduce in an altered or variant form or in a different chromosomal location. A transgene can include one or more transcriptional regulatory sequences and any other nucleic acid, such as introns, that may be useful or necessary for optimal expression of a selected nucleic acid. A transgene can be as few as a couple of nucleotides long, but is preferably at least about 50, 100, 150, 200, 250, 300, 350, 400, or 500 nucleotides long or even longer and can be, e.g., an entire genome. A transgene can be coding or non-coding sequences, or a combination thereof. A transgene usually comprises a regulatory element that is capable of driving the expression of one or more transgenes under appropriate conditions. By “transgenic animal” is meant an animal comprising a transgene as described above. Transgenic animals are made by techniques that are well known in the art. The disclosed nucleic acids, in whole or in part, in any combination, can be transgenes as disclosed herein.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein.

The disclosed transgenic animals can be any non-human animal, including a non-human mammal (e.g., mouse, rat, rabbit, squirrel, hamster, rabbits, guinea pigs, pigs, micro-pigs, prairie dogs, baboons, squirrel monkeys and chimpanzees, etc), bird or an amphibian, in which one or more cells contain heterologous nucleic acid introduced by way of human intervention, such as by transgenic techniques well known in the art. For example, the animal can be selected from the group consisting of avian, bovine, canine, caprine, equine, feline, leporine, murine, ovine, porcine, non-human primate. Thus, the animal can be a mouse, dog or cat. Thus, the animal can be a rodent.

Generally, the nucleic acid is introduced into the cell, directly or indirectly, by introduction into a precursor of the cell, such as by microinjection or by infection with a recombinant virus. The disclosed transgenic animals can also include the progeny of animals which had been directly manipulated or which were the original animal to receive one or more of the disclosed nucleic acids. This molecule may be integrated within a chromosome, or it may be extrachromosomally replicating DNA. For techniques related to the production of transgenic animals, see, inter alia, Hogan et al (1986) Manipulating the Mouse Embryo—A Laboratory Manual Cold Spring Harbor Laboratory, Cold Spring Harbor, N.Y., 1986).

Animals suitable for transgenic experiments can be obtained from standard commercial sources such as Charles River (Wilmington, Mass.), Taconic (Germantown, N.Y.), and Harlan Sprague Dawley (Indianapolis, Ind.). For example, if the transgenic animal is a mouse, many mouse strains are suitable, but C57BL/6 female mice can be used for embryo retrieval and transfer. C57BL/6 males can be used for mating and vasectomized C57BL/6 studs can be used to stimulate pseudopregnancy. Vasectomized mice and rats can be obtained from the supplier. Transgenic animals can be made by any known procedure, including microinjection methods, and embryonic stem cells methods. The procedures for manipulation of the rodent embryo and for microinjection of DNA are described in detail in Hogan et al., Manipulating the Mouse Embryo (Cold Spring Harbor Laboratory, Cold Spring Harbor, N.Y., 1986), the teachings of which are generally known and are incorporated herein.

Transgenic animals can be identified by analyzing their DNA. For this purpose, for example, when the transgenic animal is an animal with a tail, such as rodent, tail samples (1 to 2 cm) can be removed from three week old animals. DNA from these or other samples can then be prepared and analyzed, for example, by Southern blot, PCR, or slot blot to detect transgenic founder (F (0)) animals and their progeny (F (1) and F (2)). Thus, also provided are transgenic non-human animals that are progeny of crosses between a transgenic animal of the invention and a second animal. Transgenic animals can be bred with other transgenic animals, where the two transgenic animals were generated using different transgenes, to test the effect of one gene product on another gene product or to test the combined effects of two gene products.

The disclosed non-human animal and methods of making same obviate the need to immunocomprimise the animal. Thus, in some aspects, the disclosed non-human animal is not immunocompromised. Thus, in some aspects, the disclosed non-human animal is not a nude mouse.

i. Phenotype

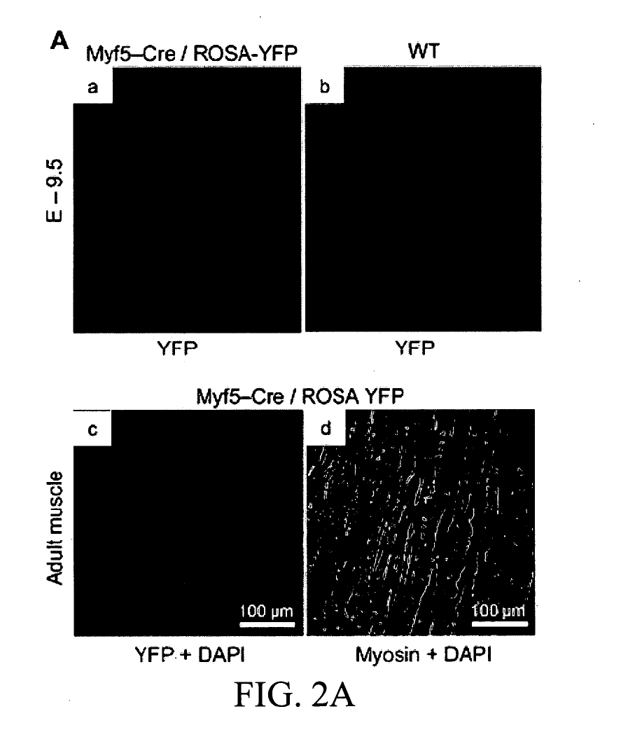

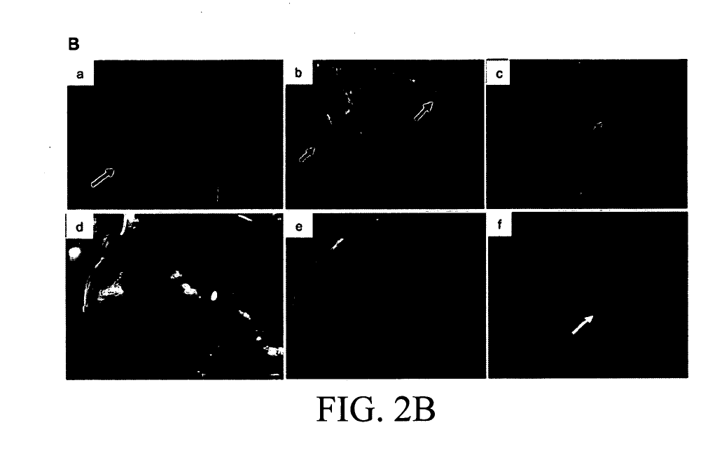

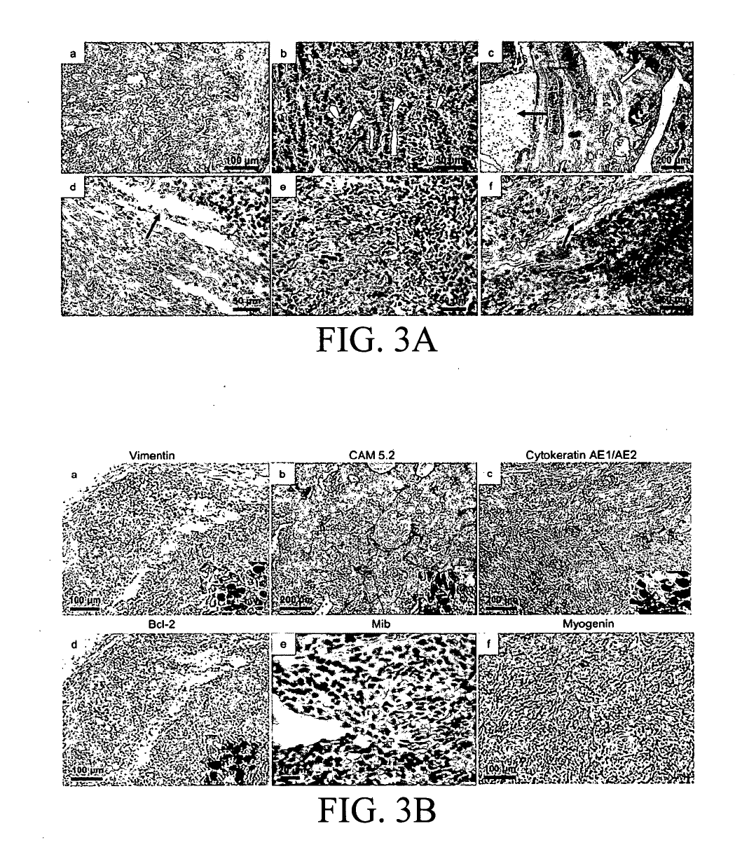

The herein disclosed non-human animal models can comprise vascularized tumors. These tumors can affect the musculatures of the limb near joints and the intercostal region (FIGS. 2Ba, 2Bb, and 2Bd and Table 3). The tumors can be hemorrhagic (FIG. 3Ac, white arrow), with cystic spaces often detected within larger tumors (FIG. 3Ac, black arrow). The tumors can have biphasic (FIGS. 3Aa-3Ac) and/or monophasic variants (FIG. 3Ad). For example, the animal can comprise monophasic tumors containing trapped skeletal muscle fibers (FIG. 3Ad, black arrow). The animal can comprise biphasic tumors with epithelioid cells arranged in a glandular pattern amid spindle cells (FIG. 3Ab, black arrow).

The tumors generated in the disclosed model can show expression of epithelial cytokeratins (positive for cytokeratin AE 1/AE2 cocktail and CAM5.2) as well as mesenchymal marker vimentin (FIGS. 3Ba-3Bd). The tumors generated in the disclosed model can show widespread expression of the proliferation marker Mib (FIG. 3Be). The tumors generated in the disclosed model can be negative for myogenin (FIG. 3Bf).

4. Expression Control

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

The disclosed SYT-SSX fusion polypeptide can be expressed in one or more cell/tissue types. Thus, the one or more cells of the non-human animal can comprise a nucleic acid encoding a SYT-SSX fusion polypeptide operably linked to an expression control sequence. For example, expression control sequence can be a constitutive promoter. The expression control sequence can be a heterologous promoter. The expression control sequence can be an inducible promoter. The expression control sequence can be tissue specific promoter.

The disclosed SYT-SSX fusion polypeptide can be expressed in myogenic cells. Thus, the one or more myogenic cells of the non-human animal can comprise a nucleic acid encoding a SYT-SSX fusion polypeptide operably linked to a myogenic-specific expression control sequence. For example, the myogenic-specific expression control sequence can be the Myf5 promoter, MyoD promoter, or MyoG (myogenin) promoter. Other potential myogenic promoters include Pax3, Pax7, and Myf6. In some aspects, the myogenic-specific expression control sequence is not Pax3, Pax7, and Myf6.

Activity of the expression control sequence can be regulated by a transactivator. For example, the cells of the non-human animal can comprise a first and second polynucleotide, wherein the first polynucleotide comprises a nucleic acid sequence encoding a SYT-SSX fusion polypeptide operably linked to a first expression control sequence and a transcriptional termination signal, wherein the transcription termination signal substantially prevents expression of the SYT-SSX fusion polypeptide, and the second polynucleotide comprises a nucleic acid encoding a transactivator polypeptide operably linked to an expression control sequence (such as a myogenic-specific expression control sequence), wherein expression of the transactivator polypeptide abolishes the effect of the transcription termination signal to substantially prevent expression of the SYT-SSX fusion polypeptide, wherein the non-human animal comprises synovial sarcomas. The first expression control sequence of the second example can be 5′ to the transcriptional termination signal, and wherein transcriptional termination signal is 5′ to the nucleic acid sequence encoding a SYT-SSX fusion polypeptide. For example, the first polynucleotides can have the structures:
1) Pr₁- [symbol] STOP [symbol] -SYT-SSX2, and
2) Pr₂-TA,
   wherein,

Pr₁ and Pr₂ are expression control sequences,

STOP is a transcriptional termination signal,

TA is a transactivator polypeptide, and

[symbol] is the target of the transactivator polypeptide to abolish the effect of the transcriptional termination signal.

Expression of the SYT-SSX fusion polypeptide in the disclosed non-human animal can be inducible. Thus, the cells of the non-human animal can comprise a first and second polynucleotide, wherein the first polynucleotide comprises a nucleic acid sequence encoding a SYT-SSX fusion polypeptide operably linked to an expression control sequence (such as a myogenic-specific expression control sequence) and a transcriptional termination signal, wherein the transcription termination signal substantially prevents expression of the SYT-SSX fusion polypeptide, and the second polynucleotide comprises a nucleic acid encoding a transactivator polypeptide operably linked to an inducible promoter wherein expression of the transactivator polypeptide abolishes the effect of the transcription termination signal to substantially prevent expression of the SYT-SSX fusion polypeptide, wherein the non-human animal comprises synovial sarcomas. Thus, for example, Pr₂ in the above system can be an inducible promoter.

Alternatively, the cells of the non-human animal can comprise a first, second, and third polynucleotide, wherein the first polynucleotide comprises a nucleic acid sequence encoding a SYT-SSX fusion polypeptide operably linked to a first expression control sequence and a first transcriptional termination signal, wherein the first transcription termination signal substantially prevents expression of the SYT-SSX fusion polypeptide; the second polynucleotide comprises a nucleic acid encoding a first transactivator polypeptide operably linked to an expression control sequence (such as a myogenic-specific expression control sequence) and a second transcriptional termination signal, wherein the second transcription termination signal substantially prevents expression of the first transactivator polypeptide; and the third polynucleotide comprises a nucleic acid encoding a second transactivator polypeptide operably linked to an inducible promoter wherein expression of the second transactivator polypeptide abolishes the effect of the second transcription termination signal to substantially prevent expression of the first transactivator protein, wherein the non-human animal comprises synovial sarcomas. For example, the first polynucleotides can have the structures:
1) Pr₁- [symbol] STOP [symbol] -SYT-SSX2, and
2) Pr₂- [symbol] STOP [symbol] -TA₁,
3) Pr₃-TA₂
   wherein,

Pr₁, and Pr₂ are expression control sequences,

wherein Pr₃ is an inducible expression control sequence,

STOP is a transcriptional termination signal,

TA is a transactivator polypeptide,

[symbol] is the target of the first transactivator polypeptide (TA₁), and

[symbol] is the target of the second transactivator polypeptide (TA₂).

In some aspects, the transactivator itself can be inducible. For example, the activity of the transactivator can be controlled by the presence or absence of a compound. For example, in order to control temporal activity of the Cre excision reaction, forms of Cre which take advantage of various ligand binding domains have been developed. One successful strategy for inducing temporally specific Cre activity involves fusing the enzyme with a mutated ligand-binding domain of the human estrogen receptor (ER). Upon the introduction of the drug tamoxifen (an estrogen receptor antagonist), the Cre-ER construct is able to penetrate the nucleus and induce targeted mutation. ER binds tamoxifen with greater affinity than endogenous estrogens, which allows Cre-ER to remain cytoplasmic in animals untreated with tamoxifen. The temporal control of site-specific recombinase activity by tamoxifen permits genetic changes to be induced later in embryogenesis and/or in adult tissues. This allows researchers to bypass embryonic lethality while still investigating the function of targeted genes.

i. Viral Promoters and Enhancers

The expression control sequence of the disclosed nucleic acids, such as the first expression control sequence of the above examples, can be any existing promoter that is sufficiently active in the target tissue. For example, the expression control sequence can mediate a high level of expression in the target tissue. Further, the expression control sequence can be active in most tissue/cell types.

Promoters controlling transcription from vectors in mammalian host cells may be obtained from various sources, for example, the genomes of viruses such as: polyoma, Simian Virus 40 (SV40), adenovirus, retroviruses, hepatitis-B virus and most preferably cytomegalovirus, or from heterologous mammalian promoters, e.g. beta actin promoter. The early and late promoters of the SV40 virus are conveniently obtained as an SV40 restriction fragment which also contains the SV40 viral origin of replication (Fiers et al., Nature, 273: 113 (1978)). The immediate early promoter of the human cytomegalovirus is conveniently obtained as a HindIII E restriction fragment (Greenway, P. J. et al., Gene 18: 355-360 (1982)). Of course, promoters from the host cell or related species also are useful herein.

Enhancer generally refers to a sequence of DNA that functions at no fixed distance from the transcription start site and can be either 5′ (Laimins, L. et al., Proc. Natl. Acad. Sci. 78: 993 (1981)) or 3′ (Lusky, M. L., et al., Mol. Cell. Bio. 3: 1108 (1983)) to the transcription unit. Furthermore, enhancers can be within an intron (Banerji, J. L. et al., Cell 33: 729 (1983)) as well as within the coding sequence itself (Osborne, T. F., et al., Mol. Cell. Bio. 4: 1293 (1984)). They are usually between 10 and 300 bp in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, α-fetoprotein and insulin), typically one will use an enhancer from a eukaryotic cell virus for general expression. Examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer region can act as a constitutive promoter and/or enhancer to maximize expression of the region of the transcription unit to be transcribed. In certain constructs the promoter and/or enhancer region be active in all eukaryotic cell types, even if it is only expressed in a particular type of cell at a particular time. A promoter of this type can be the CMV promoter (650 bases). Other promoters include SV40 promoters, cytomegalovirus (full length promoter), and retroviral vector LTR.

Thus, the first expression control sequence can be ROSA26 promoter. The first expression control sequence can be β-actin promoter. The first expression control sequence can be CMV promoter. The first expression control sequence can be CAG promoter. The first expression control sequence can be MC1 promoter. The first expression control sequence can be Ubc promoter. The first expression control sequence can be phosphoglycerate kinase (PGK) promoter.

ii. Myogenic Expression Control Sequence

Any expression control sequence, including promoters and enhancers, that are known or newly discovered to direct gene expression specifically in muscles, such as skeletal muscles, can be used in the disclosed compositions and methods.

For example, the myogenic-specific expression control sequence of the above examples can be the Myf5 promoter. The myogenic-specific expression control sequence of the above examples can be the MyoD promoter. The myogenic-specific expression control sequence of the above examples can be the MyoG (myogenin) promoter. The myogenic-specific expression control sequence of the above examples can be the Pax3 promoter Pax7. The myogenic-specific expression control sequence of the above examples can be the Myf6 promoter. In some aspects, the myogenic-specific expression control sequence is not Pax3 promoter. In some aspects, the myogenic-specific expression control sequence is not Pax7 promoter. In some aspects, the myogenic-specific expression control sequence is not Myf6 promoter.

In some aspects, expression can be targeted to mature myofibers via skeletal muscle specific promoters. Thus, for example, the myogenic-specific expression control sequence of the above examples can be a myosin heavy chain (MHC) promoter. The myogenic-specific expression control sequence of the above examples can be a myosin lich chain (MLC) promoter. The skilled artisan can select these promoters using routine skill based on their known expression patterns and use in expression vectors.

iii. Inducible Promoter

As their name says, the activity of inducible promoters is induced by the presence or absence of biotic or abiotic factors. Inducible promoters are a very powerful tool in genetic engineering because the expression of genes operably linked to them can be turned on or off at certain stages of development of an organism or in a particular tissue.

There are virtually hundreds of inducible promoters that vary according to the organism source and cells or tissues where they regulate gene transcription. Inducible promoters include chemically-regulated promoters, including promoters whose transcriptional activity is regulated by the presence or absence of alcohol, tetracycline, steroids, metal and other compounds, and physically-regulated promoters, including promoters whose transcriptional activity is regulated by the presence or absence of light and low or high temperatures.

The transcription activity of chemically-regulated promoters is modulated by chemical compounds that either turn off or turn on gene transcription. As prerequisites, the chemicals influencing promoter activity typically should not be naturally present in the organism where expression of the transgene is sought; should not be toxic; should affect only the expression of the gene of interest; should be of easy application or removal; and should induce a clear expression pattern of either high or very low gene expression. Preferably, chemically-regulated promoters should be derived from organisms distant in evolution to the organisms where its action is required. Thus, promoters to be used in plants are mostly derived from organisms such as yeast, E. coli, Drosophila or mammalian cells.

a. Chemically-regulated promoters

(A) Alcohol-Regulated

The promoter can be an alcohol dehydrogenase I (alcA) gene promoter and the transactivator protein can be AlcR. Different agricultural alcohol-based formulations are used to control the expression of a gene of interest linked to the alcA promoter.

(B) Tetracycline-regulated

The promoter can be a tetracycline-responsive promoter system, which can function either as an activating or repressing gene expression system in the presence of tetracycline. Some of the elements of the systems are a tetracycline repressor protein (TetR), a tetracycline operator sequence (tetO) a tetracycline transactivator fusion protein (tTA), which is the fusion of TetR and a herpes simplex virus protein 16 (VP16) activation sequence. Eukaryotic cells transformed with the transactivation systems including animal cells are part of the protected inventions.

Tetracycline Controlled Transcriptional Activation is a method of inducible expression where transcription is reversibly turned on or off in the presence of the antibiotic tetracycline or one of its derivatives (etc. doxycycline). In nature, pTet promotes TetR, the repressor, and TetA, the protein that pumps tetracycline antibiotic out of the cell. Two systems named Tet-off and Tet-on are used.

The Tet-off system for controlling expression of genes of interest in mammalian cells was developed by Professors Hermann Bujard and Manfred Gossen at the University of Heidelberg[1] This system makes use of the tetracycline transactivator (tTA) protein created by fusing one protein, TetR(tetracycline repressor), found in Escherichia coli bacteria with another protein, VP16, produced by the Herpes Simplex Virus. The tTA protein binds on DNA at a ‘tet’O operator. Once bound the ‘tet’O operator will activate a promoter coupled to the ‘tet’O operator, activating the transcription of nearby gene. Tetracycline derivatives bind tTA and render it incapable of binding to TRE sequences, therefore preventing transactivation of target genes. This expression system is also used in generation of transgenic mice, which conditionally express gene of interest.

The Tet-on system works in the opposite fashion. In that system the rtTA protein is only capable of binding the operator when bound by doxycycline. Thus the introduction of doxycyline to the system initiates the transcription of the genetic product. The tet-on system is sometimes preferred for the faster responsiveness.

Tet system has advantages over Cre, FRT and ER (estrogen receptor) conditional gene expression systems. In Cre and FRT systems, activation of knockout of the gene is irreversible once recombination is accomplished, while in Tet and ER systems it is reversible. Tet system has very tight control on expression, while ER system is somewhat leaky. However, Tet system, which depends on transcription and subsequent translation of target gene, is not as fast acting as ER system, which stabilizes the already expressed target protein upon hormone administration

Thus, disclosed is the use of a tetracycline inducible promter driving Cre expression such that tetracycline transactivator is expressed from one of the aforementioned myogenic control sequences.

(C) Steroid-regulated

The promoter can be a steroid-responsive promoter. Examples of these promoters include promoters responseive to: glucocorticoid receptor (GR); human estrogen receptor (ER); ecdysone receptors; and steroid/retinoid/thyroid receptor superfamily.

(D) Metal-regulated Promoters

The promoter can be derived from metallothionein (proteins that bind and sequester metal ionic) genes. DNA constructs having metal-regulated promoters and eukaryotic cells transformed with them are disclosed herein.

(E) Pathogenesis-related (PR)

The promoter can be pathogenesis-related. Pathogen-related (PR) proteins are induced in plants by the presence of exogenous chemicals besides pathogen infection. Salicylic acid, ethylene and benzothiadiazole (BTH) are some of the inducers of PR proteins. Promoters derived from Arabidopsis and maize PR genes are the subject matter of patents granted to Novartis and Pioneer Hi-Bred in the United States, Australia and Europe.

b. Physically-regulated promoters

The promoter can be physically-regulated. Physically-regulated promoters induced by environmental factors such as water or salt stress, anaerobiosis, temperature, illumination and wounding have potential for use in the development of plants resistant to various stress conditions. These promoters contain regulatory elements that respond to such environmental stimuli.

Temperature-induced promoters include cold- and heat-shock-induced promoters. In many cases, these promoters are able to operate under normal temperature conditions, which vary according to the organism, but when either cold or heat is applied, the promoters maintain activity. In addition, expression can be enhanced by the application of higher or lower temperature as compared to the normal temperature conditions. One of the best studied eukaryotic heat-shock systems is the one found in Drosophila (fruit fly).

5. Transcription Termination

A D is a sequence which can prevent the transcription of one or more gene sequences contained within the nucleic acid. For example, the transcription termination signal can be a stop codon. As another example, the transcription termination signal can be a nucleic acid comprising a polyadenylation signal (PolyA). Any polyadenylation signal effective as a transcriptional stop in mammalian cells can be used. For example, the transcription termination signal can be the trimer of SV40 polyA sequence as it has been shown in literature to work in this locus (ROSA locus) for insulating ROSA promoter from downstream coding sequences prior to removal by Cre. In addition, the transcription termination signal can comprise the open reading frame of a drug resistance gene that can be used as a selection marker, typically followed by a polyadenylation signal. For example, the nucleic acid comprising the PolyA can be a neomycin resistance coding sequence.

6. Transactivators

The transcription termination signal can be flanked by recombination sequences, such that in the presence of a cognate recombinase, the transcription termination signal is excised from the inflammation nucleic acid. Thus, the transactivator can be a recombinase, such as, for example, Cre recombinase or Flp recombinase, wherein the transcriptional termination signal is flanked by recombination sites, e.g., loxP-flanked “foxed” for Cre recombinase. Recombination sequences and their use are discussed herein.

U.S. Pat. No. 4,959,317 and U.S. Pat. No. 5,434,066 are incorporated herein by reference for their teaching of the use of Cre recombinase in the site-specific recombination of DNA in eukaryotic cells. The term “Cre” recombinase, as used herein, refers to a protein having an activity that is substantially similar to the site-specific recombinase activity of the Cre protein of bacteriophage P1 (Hamilton, D. L., et al., J. Mol. Biol. 178:481-486 (1984), herein incorporated by reference for its teaching of Cre recombinase). The Cre protein of bacteriophage P1 mediates site-specific recombination between specialized sequences, known as “loxP” sequences. Hoess, R., et al., Proc. Natl. Acad. Sci. USA 79:3398-3402 (1982) and Sauer, B. L., U.S. Pat. No. 4,959,317 are herein incorporated by reference for their teaching of the lox sequences. The loxP site has been shown to consist of a double-stranded 34 bp sequence:

(SEQ ID NO: 16)
5′ ATAACTTCGTATAATGTATGCTATACGAAGTTAT 3′

This sequence contains two 13 bp inverted repeat sequences which are separated from one another by an 8 bp spacer region. Other suitable lox sites include LoxB, LoxL and LoxR sites which are nucleotide sequences isolated from E. coli. These sequences are disclosed and described by Hoess et al., Proc. Natl. Acad. Sci. USA 79:3398-3402 (1982), herein incorporated by reference for the teaching of lox sites. Lox sites can also be produced by a variety of synthetic techniques which are known in the art. For example, synthetic techniques for producing lox sites are disclosed by Ito et al., Nuc. Acid Res., 10:1755 (1982) and Ogilvie et al., Science 214:270 (1981), the disclosures of which are incorporated herein by reference for their teaching of these synthetic techniques.

The Cre protein mediates recombination between two loxP sequences (Sternberg, N., et al., Cold Spring Harbor Symp. Quant. Biol. 45:297-309 (1981)). These sequences may be present on the same DNA molecule, or they may be present on different molecules. Because the internal spacer sequence of the loxP site is asymmetrical, two loxP sites can exhibit directionality relative to one another (Hoess, R. H., et al., Proc. Natl. Acad, Sci. 81:1026-1029 (1984)). Thus, when two sites on the same DNA molecule are in a directly repeated orientation, Cre will excise the DNA between the sites (Abremski, K., et al., Cell 32:1301-1311 (1983)). However, if the sites are inverted with respect to each other, the DNA between them is not excised after recombination but is simply inverted. Thus, a circular DNA molecule having two loxP sites in direct orientation will recombine to produce two smaller circles, whereas circular molecules having two loxP sites in an inverted orientation simply invert the DNA sequence flanked by the loxP sites.

Any site specific system, Cre-LoxP or Flp-FRT, comprises of two parts. The first part is the recombinase enzyme and the second part is the nucleotide recognition sequence for the recombinase. The recombinase mediates recombination between its recognition sequences. Flanking Loxp sites means that the transcriptional stop signal has the LoxP recognition sequence for Cre on either side of it. The LoxP sequences are directional in nature with two possible 5′ to 3′ orientation. In Cre-loxp system, the orientation of the Loxp sites is important. If both Loxp sites have the same orientation then after recombination, anything between the two LoxP sites will be removed. On the other hand if the LoxP sites have opposite orientation then anything in between the LoxP sites will simply be flipped instead of being removed.

Thus, the transactivator polypeptide can be Cre recombinase, wherein the transcription termination signal is flanked by LoxP. LoxP sites are considered to “flank” the transcription termination signal if they are positioned both 5′ and 3′ to the transcription termination signal. In some aspects, the LoxP sites are in the same 5′ or 3′ orientation.

Thus, also provided is a non-human mammal, wherein one or more cells of the mammal comprise a nucleic acid sequence encoding a SYT-SSX fusion polypeptide operably linked to a first expression control sequence and a transcriptional termination signal, wherein the transcription termination signal substantially prevents expression of the SYT-SSX fusion polypeptide, wherein expression of Cre recombinase by the cell alters the transcription termination signal whereby the SYT-SSX fusion polypeptide is expressed.

The expression of the transactivator can be inducible. Thus, the nucleic acid encoding the transactivator can be operably linked to an inducible expression control sequence. For example, the nucleic acid encoding the transactivator can be operably linked to a tetracycline-responsive promoter. As another example, the gene encoding the transactivator can be operably linked to the Mx1 promoter, which is activated by the presence of interferon.

The activity of the transactivator can be conditional. Thus, the transactivator can comprise a ligand binding domain, wherein the ligand for the ligand binding domain can control the activity of the trans activator.

For example, the transactivator can be CreER, which is a fusion protein comprising Cre recombinase and an estrogen receptor. Thus, translocation of CreER can be regulated by the drug tamoxifen.

The transactivator can be the CrePr fusion protein, which is a fusion protein comprising Cre recombinase and the mutated progesterone receptor hPR891. The mutated hPR891 receptor is highly sensitive to the synthetic progesterone compound mifepristone (RU486) but is unable to bind progesterone or other endogenous hormones. Thus, translocation of CrePR can be regulated by the drug Mifepristone (RU486).

Other such systems are known or can be designed to provide a transactivator, such as a site-specific recombinase, whose expression or activity is inducible.

In some aspects, the transactivator polypeptide can be under dual transcriptional (inducible) and post-translational (conditional) regulation. For example, gene expression of the transactivator (e.g., CreER or CrePR) can be inducible by the same ligand that binds the ligand binding domain of the transactivator and conditions its activity.

7. Markers

The herein disclosed nucleic acids can further comprise a nucleic acid sequence encoding a detection marker. This marker product is used to determine if the gene has been delivered to the cell and once delivered is being expressed. For example, the marker gene can be the E. Coli lacZ gene, which encodes β-galactosidase. The detection marker can be a fluorescent protein, such as green fluorescent protein.

The marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of such nutrients as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, (Southern P. and Berg, P., J. Molec. Appl. Genet. 1: 327 (1982)), mycophenolic acid, (Mulligan, R. C. and Berg, P. Science 209: 1422 (1980)) or hygromycin, (Sugden, B. et al., Mol. Cell. Biol. 5: 410-413 (1985)). The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

8. Cells

Also provided is a cell comprising a nucleic acid encoding a SYT-SSX fusion polypeptide operably linked to an expression control sequence, such as a myogenic-specific expression control sequence.

Also provided is a cell comprising a first and second polynucleotide, wherein the first polynucleotide comprises a nucleic acid sequence encoding a SYT-SSX fusion polypeptide operably linked to a first expression control sequence and a transcriptional termination signal, wherein the transcription termination signal substantially prevents expression of the SYT-SSX fusion polypeptide, and the second polynucleotide comprises a nucleic acid encoding a transactivator polypeptide operably linked to an expression control sequence, such as a myogenic-specific expression control sequence, wherein expression of the transactivator polypeptide abolishes the effect of the transcription termination signal to substantially prevent expression of the SYT-SSX fusion polypeptide.

Also provided is a cell comprising a first and second polynucleotide, wherein the first polynucleotide comprises a nucleic acid sequence encoding a SYT-SSX fusion polypeptide operably linked to an expression control sequence, such as a myogenic-specific expression control sequence and a transcriptional termination signal, wherein the transcription termination signal substantially prevents expression of the SYT-SSX fusion polypeptide, and the second polynucleotide comprises a nucleic acid encoding a transactivator polypeptide operably linked to an inducible promoter wherein expression of the transactivator polypeptide abolishes the effect of the transcription termination signal to substantially prevent expression of the SYT-SSX fusion polypeptide.

Also provided is a cell comprising a first, second, and third polynucleotide, wherein the first polynucleotide comprises a nucleic acid sequence encoding a SYT-SSX fusion polypeptide operably linked to a first expression control sequence and a first transcriptional termination signal, wherein the first transcription termination signal substantially prevents expression of the SYT-SSX fusion polypeptide; the second polynucleotide comprises a nucleic acid encoding a first transactivator polypeptide operably linked to an expression control sequence, such as a myogenic-specific expression control sequence and a second transcriptional termination signal, wherein the second transcription termination signal substantially prevents expression of the first transactivator polypeptide; and the third polynucleotide comprises a nucleic acid encoding a second transactivator polypeptide operably linked to an inducible promoter wherein expression of the second transactivator polypeptide abolishes the effect of the second transcription termination signal to substantially prevent expression of the first transactivator protein.

Cells of the human body include Keratinizing Epithelial Cells, Epidermal keratinocyte (differentiating epidermal cell), Epidermal basal cell (stem cell), Keratinocyte of fingernails and toenails, Nail bed basal cell (stem cell), Medullary hair shaft cell, Cortical hair shaft cell, Cuticular hair shaft cell, Cuticular hair root sheath cell, Hair root sheath cell of Huxley's layer, Hair root sheath cell of Henle's layer, External hair root sheath cell, Hair matrix cell (stem cell), Wet Stratified Barrier Epithelial Cells, Surface epithelial cell of stratified squamous epithelium of cornea, tongue, oral cavity, esophagus, anal canal, distal urethra and vagina, basal cell (stem cell) of epithelia of cornea, tongue, oral cavity, esophagus, anal canal, distal urethra and vagina, Urinary epithelium cell (lining bladder and urinary ducts), Exocrine Secretory Epithelial Cells, Salivary gland mucous cell (polysaccharide-rich secretion), Salivary gland serous cell (glycoprotein enzyme-rich secretion), Von Ebner's gland cell in tongue (washes taste buds), Mammary gland cell (milk secretion), Lacrimal gland cell (tear secretion), Ceruminous gland cell in ear (wax secretion), Eccrine sweat gland dark cell (glycoprotein secretion), Eccrine sweat gland clear cell (small molecule secretion), Apocrine sweat gland cell (odoriferous secretion, sex-hormone sensitive), Gland of Moll cell in eyelid (specialized sweat gland), Sebaceous gland cell (lipid-rich sebum secretion), Bowman's gland cell in nose (washes olfactory epithelium), Brunner's gland cell in duodenum (enzymes and alkaline mucus), Seminal vesicle cell (secretes seminal fluid components, including fructose for swimming sperm), Prostate gland cell (secretes seminal fluid components), Bulbourethral gland cell (mucus secretion), Bartholin's gland cell (vaginal lubricant secretion), Gland of Littre cell (mucus secretion), Uterus endometrium cell (carbohydrate secretion), Isolated goblet cell of respiratory and digestive tracts (mucus secretion), Stomach lining mucous cell (mucus secretion), Gastric gland zymogenic cell (pepsinogen secretion), Gastric gland oxyntic cell (HCl secretion), Pancreatic acinar cell (bicarbonate and digestive enzyme secretion), Paneth cell of small intestine (lysozyme secretion), Type II pneumocyte of lung (surfactant secretion), Clara cell of lung, Hormone Secreting Cells, Anterior pituitary cell secreting growth hormone, Anterior pituitary cell secreting follicle-stimulating hormone, Anterior pituitary cell secreting luteinizing hormone, Anterior pituitary cell secreting prolactin, Anterior pituitary cell secreting adrenocorticotropic hormone, Anterior pituitary cell secreting thyroid-stimulating hormone, Intermediate pituitary cell secreting melanocyte-stimulating hormone, Posterior pituitary cell secreting oxytocin, Posterior pituitary cell secreting vasopressin, Gut and respiratory tract cell secreting serotonin, Gut and respiratory tract cell secreting endorphin, Gut and respiratory tract cell secreting somatostatin, Gut and respiratory tract cell secreting gastrin, Gut and respiratory tract cell secreting secretin, Gut and respiratory tract cell secreting cholecystokinin, Gut and respiratory tract cell secreting insulin, Gut and respiratory tract cell secreting glucagon, Gut and respiratory tract cell secreting bombesin, Thyroid gland cell secreting thyroid hormone, Thyroid gland cell secreting calcitonin, Parathyroid gland cell secreting parathyroid hormone, Parathyroid gland oxyphil cell, Adrenal gland cell secreting epinephrine, Adrenal gland cell secreting norepinephrine, Adrenal gland cell secreting steroid hormones (mineralcorticoids and gluco corticoids), Leydig cell of testes secreting testosterone, Theca interna cell of ovarian follicle secreting estrogen, Corpus luteum cell of ruptured ovarian follicle secreting progesterone, Kidney juxtaglomerular apparatus cell (renin secretion), Macula densa cell of kidney, Peripolar cell of kidney, Mesangial cell of kidney, Epithelial Absorptive Cells (Gut, Exocrine Glands and Urogenital Tract), Intestinal brush border cell (with microvilli), Exocrine gland striated duct cell, Gall bladder epithelial cell, Kidney proximal tubule brush border cell, Kidney distal tubule cell, Ductulus efferens nonciliated cell, Epididymal principal cell, Epididymal basal cell, Metabolism and Storage Cells, Hepatocyte (liver cell), White fat cell, Brown fat cell, Liver lipocyte, Barrier Function Cells (Lung, Gut, Exocrine Glands and Urogenital Tract), Type I pneumocyte (lining air space of lung), Pancreatic duct cell (centroacinar cell), Nonstriated duct cell (of sweat gland, salivary gland, mammary gland, etc.), Kidney glomerulus parietal cell, Kidney glomerulus podocyte, Loop of Henle thin segment cell (in kidney), Kidney collecting duct cell, Duct cell (of seminal vesicle, prostate gland, etc.), Epithelial Cells Lining Closed Internal Body Cavities, Blood vessel and lymphatic vascular endothelial fenestrated cell, Blood vessel and lymphatic vascular endothelial continuous cell, Blood vessel and lymphatic vascular endothelial splenic cell, Synovial cell (lining joint cavities, hyaluronic acid secretion), Serosal cell (lining peritoneal, pleural, and pericardial cavities), Squamous cell (lining perilymphatic space of ear), Squamous cell (lining endolymphatic space of ear), Columnar cell of endolymphatic sac with microvilli (lining endolymphatic space of ear), Columnar cell of endolymphatic sac without microvilli (lining endolymphatic space of ear), Dark cell (lining endolymphatic space of ear), Vestibular membrane cell (lining endolymphatic space of ear), Stria vascularis basal cell (lining endolymphatic space of ear), Stria vascularis marginal cell (lining endolymphatic space of ear), Cell of Claudius (lining endolymphatic space of ear), Cell of Boettcher (lining endolymphatic space of ear), Choroid plexus cell (cerebrospinal fluid secretion), Pia-arachnoid squamous cell, Pigmented ciliary epithelium cell of eye, Nonpigmented ciliary epithelium cell of eye, Corneal endothelial cell, Ciliated Cells with Propulsive Function, Respiratory tract ciliated cell, Oviduct ciliated cell (in female), Uterine endometrial ciliated cell (in female), Rete testis cilated cell (in male), Ductulus efferens ciliated cell (in male), Ciliated ependymal cell of central nervous system (lining brain cavities), Extracellular Matrix Secretion Cells, Ameloblast epithelial cell (tooth enamel secretion), Planum semilunatum epithelial cell of vestibular apparatus of ear (proteoglycan secretion), Organ of Corti interdental epithelial cell (secreting tectorial membrane covering hair cells), Loose connective tissue fibroblasts, Corneal fibroblasts, Tendon fibroblasts, Bone marrow reticular tissue fibroblasts, Other (nonepithelial) fibroblasts, Blood capillary pericyte, Nucleus pulposus cell of intervertebral disc, Cementoblast/cementocyte (tooth root bonelike cementum secretion), Odontoblast/odontocyte (tooth dentin secretion), Hyaline cartilage chondrocyte, Fibrocartilage chondrocyte, Elastic cartilage chondrocyte, Osteoblast/osteocyte, Osteoprogenitor cell (stem cell of osteoblasts), Hyalocyte of vitreous body of eye, Stellate cell of perilymphatic space of ear, Contractile Cells, Red skeletal muscle cell (slow), White skeletal muscle cell (fast), Intermediate skeletal muscle cell, Muscle spindle—nuclear bag cell, Muscle spindle—nuclear chain cell, Satellite cell (stem cell), Ordinary heart muscle cell, Nodal heart muscle cell, Purkinje fiber cell, Smooth muscle cell (various types), Myoepithelial cell of iris, Myoepithelial cell of exocrine glands, Blood and Immune System Cells, Erythrocyte (red blood cell), Megakaryocyte, Monocyte, Connective tissue macrophage (various types), Epidermal Langerhans cell, Osteoclast (in bone), Dendritic cell (in lymphoid tissues), Microglial cell (in central nervous system), Neutrophil, Eosinophil, Basophil, Mast cell, Helper T lymphocyte cell, Suppressor T lymphocyte cell, Killer T lymphocyte cell, IgM B lymphocyte cell, IgG B lymphocyte cell, IgA B lymphocyte cell, IgE B lymphocyte cell, Killer cell, Stem cells and committed progenitors for the blood and immune system (various types), Sensory Transducer Cells, Photoreceptor rod cell of eye, Photoreceptor blue-sensitive cone cell of eye, Photoreceptor green-sensitive cone cell of eye, Photoreceptor red-sensitive cone cell of eye, Auditory inner hair cell of organ of Corti, Auditory outer hair cell of organ of Corti, Type I hair cell of vestibular apparatus of ear (acceleration and gravity), Type II hair cell of vestibular apparatus of ear (acceleration and gravity), Type I taste bud cell, Olfactory neuron, Basal cell of olfactory epithelium (stem cell for olfactory neurons), Type I carotid body cell (blood pH sensor), Type II carotid body cell (blood pH sensor), Merkel cell of epidermis (touch sensor), Touch-sensitive primary sensory neurons (various types), Cold-sensitive primary sensory neurons, Heat-sensitive primary sensory neurons, Pain-sensitive primary sensory neurons (various types), Proprioceptive primary sensory neurons (various types), Autonomic Neuron Cells, Cholinergic neural cell (various types), Adrenergic neural cell (various types), Peptidergic neural cell (various types), Sense Organ and Peripheral Neuron Supporting Cells, Inner pillar cell of organ of Corti, Outer pillar cell of organ of Corti, Inner phalangeal cell of organ of Corti, Outer phalangeal cell of organ of Corti, Border cell of organ of Corti, Hensen cell of organ of Corti, Vestibular apparatus supporting cell, Type I taste bud supporting cell, Olfactory epithelium supporting cell, Schwann cell, Satellite cell (encapsulating peripheral nerve cell bodies), Enteric glial cell, Central Nervous System Neurons and Glial Cells, Neuron cell (large variety of types, still poorly classified), Astrocyte glial cell (various types), Oligodendrocyte glial cell, Lens Cells, Anterior lens epithelial cell, Crystallin-containing lens fiber cell, Pigment Cells, Melanocyte, Retinal pigmented epithelial cell, Germ Cells, Oogonium/oocyte, Spermatocyte, Spermatogonium cell (stem cell for spermatocyte), Nurse Cells, Ovarian follicle cell, Sertoli cell (in testis), and Thymus epithelial cell.

9. Nucleic Acids

There are a variety of molecules disclosed herein that are nucleic acid based. The disclosed nucleic acids can be made up of for example, nucleotides, nucleotide analogs, or nucleotide substitutes. Non-limiting examples of these and other molecules are discussed herein. It is understood that for example, when a vector is expressed in a cell, the expressed mRNA will typically be made up of A, C, G, and U. Likewise, it is understood that if, for example, an antisense molecule is introduced into a cell or cell environment through for example exogenous delivery, it is advantagous that the antisense molecule be made up of nucleotide analogs that reduce the degradation of the antisense molecule in the cellular environment.

i. Nucleotides and related molecules

A nucleotide is a molecule that contains a base moiety, a sugar moiety and a phosphate moiety. Nucleotides can be linked together through their phosphate moieties and sugar moieties creating an internucleoside linkage. The base moiety of a nucleotide can be adenin-9-yl (A), cytosin-1-yl (C), guanin-9-yl (G), uracil-1-yl (U), and thymin-1-yl (T). The sugar moiety of a nucleotide is a ribose or a deoxyribose. The phosphate moiety of a nucleotide is pentavalent phosphate. An non-limiting example of a nucleotide would be 3′-AMP (3′-adenosine monophosphate) or 5′-GMP (5′-guanosine monophosphate). There are many varieties of these types of molecules available in the art and available herein.

A nucleotide analog is a nucleotide which contains some type of modification to either the base, sugar, or phosphate moieties. Modifications to nucleotides are well known in the art and would include for example, 5-methylcytosine (5-me-C), 5-hydroxymethyl cytosine, xanthine, hypoxanthine, and 2-aminoadenine as well as modifications at the sugar or phosphate moieties. There are many varieties of these types of molecules available in the art and available herein.

Nucleotide substitutes are molecules having similar functional properties to nucleotides, but which do not contain a phosphate moiety, such as peptide nucleic acid (PNA). Nucleotide substitutes are molecules that will recognize nucleic acids in a Watson-Crick or Hoogsteen manner, but which are linked together through a moiety other than a phosphate moiety. Nucleotide substitutes are able to conform to a double helix type structure when interacting with the appropriate target nucleic acid. There are many varieties of these types of molecules available in the art and available herein.

It is also possible to link other types of molecules (conjugates) to nucleotides or nucleotide analogs to enhance for example, cellular uptake. Conjugates can be chemically linked to the nucleotide or nucleotide analogs. Such conjugates include but are not limited to lipid moieties such as a cholesterol moiety. (Letsinger et al., Proc. Natl. Acad. Sci. USA, 1989, 86, 6553-6556). There are many varieties of these types of molecules available in the art and available herein.

A Watson-Crick interaction is at least one interaction with the Watson-Crick face of a nucleotide, nucleotide analog, or nucleotide substitute. The Watson-Crick face of a nucleotide, nucleotide analog, or nucleotide substitute includes the C2, N1, and C6 positions of a purine based nucleotide, nucleotide analog, or nucleotide substitute and the C2, N3, C4 positions of a pyrimidine based nucleotide, nucleotide analog, or nucleotide substitute.

A Hoogsteen interaction is the interaction that takes place on the Hoogsteen face of a nucleotide or nucleotide analog, which is exposed in the major groove of duplex DNA. The Hoogsteen face includes the N7 position and reactive groups (NH2 or O) at the C6 position of purine nucleotides.

ii. Sequences

There are a variety of sequences related to the protein molecules involved in the signaling pathways disclosed herein, all of which are encoded by nucleic acids or are nucleic acids. The sequences for the human analogs of these genes, as well as other anlogs, and alleles of these genes, and splice variants and other types of variants, are available in a variety of protein and gene databases, including Genbank. Those sequences available at the time of filing this application at Genbank are herein incorporated by reference in their entireties as well as for individual subsequences contained therein. Genbank can be accessed at www.ncbi.nih.gov/entrez/query.fcgi. Those of skill in the art understand how to resolve sequence discrepancies and differences and to adjust the compositions and methods relating to a particular sequence to other related sequences. Primers and/or probes can be designed for any given sequence given the information disclosed herein and known in the art.

10. Peptides

i. Protein variants

As discussed herein there are numerous variants and derivativesof peptides that are known and herein contemplated. Protein variants and derivatives are well understood to those of skill in the art and in can involve amino acid sequence modifications. For example, amino acid sequence modifications typically fall into one or more of three classes: substitutional, insertional or deletional variants. Insertions include amino and/or carboxyl terminal fusions as well as intrasequence insertions of single or multiple amino acid residues. Insertions ordinarily will be smaller insertions than those of amino or carboxyl terminal fusions, for example, on the order of one to four residues. Immunogenic fusion protein derivatives, such as those described in the examples, are made by fusing a polypeptide sufficiently large to confer immunogenicity to the target sequence by cross-linking in vitro or by recombinant cell culture transformed with DNA encoding the fusion. Deletions are characterized by the removal of one or more amino acid residues from the protein sequence. Typically, no more than about from 2 to 6 residues are deleted at any one site within the protein molecule. These variants ordinarily are prepared by site specific mutagenesis of nucleotides in the DNA encoding the protein, thereby producing DNA encoding the variant, and thereafter expressing the DNA in recombinant cell culture. Techniques for making substitution mutations at predetermined sites in DNA having a known sequence are well known, for example M13 primer mutagenesis and PCR mutagenesis. Amino acid substitutions are typically of single residues, but can occur at a number of different locations at once; insertions usually will be on the order of about from 1 to 10 amino acid residues; and deletions will range about from 1 to 30 residues. Deletions or insertions preferably are made in adjacent pairs, i.e. a deletion of 2 residues or insertion of 2 residues. Substitutions, deletions, insertions or any combination thereof may be combined to arrive at a final construct. The mutations must not place the sequence out of reading frame and preferably will not create complementary regions that could produce secondary mRNA structure. Substitutional variants are those in which at least one residue has been removed and a different residue inserted in its place. Such substitutions generally are made in accordance with the following Table 1 and are referred to as conservative substitutions.

TABLE 1
Amino Acid Substitutions
Original Residue Exemplary Conservative
Substitutions, others are known in the art.

| | |
|---|---|
| Ala | Ser |
| Arg | Lys; Gln |
| Asn | Gln; His |
| Asp | Glu |
| Cys | Ser |
| Gln | Asn, Lys |
| Glu | Asp |
| Gly | Pro |
| His | Asn; Gln |
| Ile | Leu; Val |
| Leu | Ile; Val |
| Lys | Arg; Gln |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Substantial changes in function or immunological identity are made by selecting substitutions that are less conservative than those in Table 1, i.e., selecting residues that differ more significantly in their effect on maintaining (a) the structure of the polypeptide backbone in the area of the substitution, for example as a sheet or helical conformation, (b) the charge or hydrophobicity of the molecule at the target site or (c) the bulk of the side chain. The substitutions which in general are expected to produce the greatest changes in the protein properties will be those in which (a) a hydrophilic residue, e.g. seryl or threonyl, is substituted for (or by) a hydrophobic residue, e.g. leucyl, isoleucyl, phenylalanyl, valyl or alanyl; (b) a cysteine or proline is substituted for (or by) any other residue; (c) a residue having an electropositive side chain, e.g., lysyl, arginyl, or histidyl, is substituted for (or by) an electronegative residue, e.g., glutamyl or aspartyl; or (d) a residue having a bulky side chain, e.g., phenylalanine, is substituted for (or by) one not having a side chain, e.g., glycine, in this case, (e) by increasing the number of sites for sulfation and/or glycosylation.

For example, the replacement of one amino acid residue with another that is biologically and/or chemically similar is known to those skilled in the art as a conservative substitution. For example, a conservative substitution would be replacing one hydrophobic residue for another, or one polar residue for another. The substitutions include combinations such as, for example, Gly, Ala; Val, Ile, Leu; Asp, Glu; Asn, Gln; Ser, Thr; Lys, Arg; and Phe, Tyr. Such conservatively substituted variations of each explicitly disclosed sequence are included within the mosaic polypeptides provided herein.

Substitutional or deletional mutagenesis can be employed to insert sites for N-glycosylation (Asn-X-Thr/Ser) or O-glycosylation (Ser or Thr). Deletions of cysteine or other labile residues also may be desirable. Deletions or substitutions of potential proteolysis sites, e.g. Arg, is accomplished for example by deleting one of the basic residues or substituting one by glutaminyl or histidyl residues.

Certain post-translational derivatizations are the result of the action of recombinant host cells on the expressed polypeptide. Glutaminyl and asparaginyl residues are frequently post-translationally deamidated to the corresponding glutamyl and asparyl residues. Alternatively, these residues are deamidated under mildly acidic conditions. Other post-translational modifications include hydroxylation of proline and lysine, phosphorylation of hydroxyl groups of seryl or threonyl residues, methylation of the o-amino groups of lysine, arginine, and histidine side chains (T. E. Creighton, Proteins: Structure and Molecular Properties, W.H. Freeman & Co., San Francisco pp 79-86 [1983]), acetylation of the N-terminal amine and, in some instances, amidation of the C-terminal carboxyl.

It is understood that one way to define the variants and derivatives of the disclosed proteins herein is through defining the variants and derivatives in terms of homology/identity to specific known sequences. For example, SEQ ID NO:2 sets forth a particular amino acid sequence of SYT-SSX for use in the disclosed non-human animals. Specifically disclosed are variants of these and other proteins herein disclosed which have at least, 70% or 75% or 80% or 85% or 90% or 95% homology to the stated sequence. Those of skill in the art readily understand how to determine the homology of two proteins. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment.

It is understood that the description of conservative mutations and homology can be combined together in any combination, such as embodiments that have at least 70% homology to a particular sequence wherein the variants are conservative mutations.

As this specification discusses various proteins and protein sequences it is understood that the nucleic acids that can encode those protein sequences are also disclosed. This would include all degenerate sequences related to a specific protein sequence, i.e. all nucleic acids having a sequence that encodes one particular protein sequence as well as all nucleic acids, including degenerate nucleic acids, encoding the disclosed variants and derivatives of the protein sequences. Thus, while each particular nucleic acid sequence may not be written out herein, it is understood that each and every sequence is in fact disclosed and described herein through the disclosed protein sequence. It is also understood that while no amino acid sequence indicates what particular DNA sequence encodes that protein within an organism, where particular variants of a disclosed protein are disclosed herein, the known nucleic acid sequence that encodes that protein in the particular species from which that protein arises is also known and herein disclosed and described.

It is understood that there are numerous amino acid and peptide analogs which can be incorporated into the disclosed compositions. For example, there are numerous D amino acids or amino acids which have a different functional substituent then the amino acids shown in Table 1. The opposite stereo isomers of naturally occurring peptides are disclosed, as well as the stereo isomers of peptide analogs. These amino acids can readily be incorporated into polypeptide chains by charging tRNA molecules with the amino acid of choice and engineering genetic constructs that utilize, for example, amber codons, to insert the analog amino acid into a peptide chain in a site specific way (Thorson et al., Methods in Molec. Biol. 77:43-73 (1991), Zoller, Current Opinion in Biotechnology, 3:348-354 (1992); Ibba, Biotechnology & Genetic Enginerring Reviews 13:197-216 (1995), Cahill et al., TIBS, 14(10):400-403 (1989); Benner, TIB Tech, 12:158-163 (1994); Ibba and Hennecke, Bio/technology, 12:678-682 (1994) all of which are herein incorporated by reference at least for material related to amino acid analogs).

Molecules can be produced that resemble peptides, but which are not connected via a natural peptide linkage. For example, linkages for amino acids or amino acid analogs can include CH₂NH—, —CH₂S—, —CH₂—CH₂—, —CH═CH—(cis and trans), —COCH₂—, —CH(OH)CH₂—, and —CHH₂SO— (These and others can be found in Spatola, A. F. in Chemistry and Biochemistry of Amino Acids, Peptides, and Proteins, B. Weinstein, eds., Marcel Dekker, New York, p. 267 (1983); Spatola, A. F., Vega Data (March 1983), Vol. 1, Issue 3, Peptide Backbone Modifications (general review); Morley, Trends Pharm Sci (1980) pp. 463-468; Hudson, D. et al., Int J Pept Prot Res 14:177-185 (1979) (—CH₂NH—, CH₂CH₂—); Spatola et al. Life Sci 38:1243-1249 (1986) (—CH H₂—S); Hann J. Chem. Soc Perkin Trans. 1307-314 (1982) (—CH—CH—, cis and trans); Almquist et al. J. Med. Chem. 23:1392-1398 (1980) (—COCH₂—); Jennings-White et al. Tetrahedron Lett 23:2533 (1982) (—COCH₂—); Szelke et al. European Appin, EP 45665 CA (1982): 97:39405 (1982) (—CH(OH)CH₂—); Holladay et al. Tetrahedron. Lett 24:4401-4404 (1983) (—C(OH)CH₂—); and Hruby Life Sci 31:189-199 (1982) (—CH₂—S—); each of which is incorporated herein by reference. An example non-peptide linkage is —CH₂NH—. It is understood that peptide analogs can have more than one atom between the bond atoms, such as b-alanine, g-aminobutyric acid, and the like.

Amino acid analogs and analogs and peptide analogs often have enhanced or desirable properties, such as, more economical production, greater chemical stability, enhanced pharmacological properties (half-life, absorption, potency, efficacy, etc.), altered specificity (e.g., a broad-spectrum of biological activities), reduced antigenicity, and others.

D-amino acids can be used to generate more stable peptides, because D amino acids are not recognized by peptidases and such. Systematic substitution of one or more amino acids of a consensus sequence with a D-amino acid of the same type (e.g., D-lysine in place of L-lysine) can be used to generate more stable peptides. Cysteine residues can be used to cyclize or attach two or more peptides together. This can be beneficial to constrain peptides into particular conformations. (Rizo and Gierasch Ann. Rev. Biochem. 61:387 (1992), incorporated herein by reference).

11. Sequence Similarities

It is understood that as discussed herein the use of the terms homology and identity mean the same thing as similarity. Thus, for example, if the use of the word homology is used between two non-natural sequences it is understood that this is not necessarily indicating an evolutionary relationship between these two sequences, but rather is looking at the similarity or relatedness between their nucleic acid sequences. Many of the methods for determining homology between two evolutionarily related molecules are routinely applied to any two or more nucleic acids or proteins for the purpose of measuring sequence similarity regardless of whether they are evolutionarily related or not.

In general, it is understood that one way to define any known variants and derivatives or those that might arise, of the disclosed genes and proteins herein, is through defining the variants and derivatives in terms of homology to specific known sequences. This identity of particular sequences disclosed herein is also discussed elsewhere herein. In general, variants of genes and proteins herein disclosed typically have at least, about 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99 percent homology to the stated sequence or the native sequence. Those of skill in the art readily understand how to determine the homology of two proteins or nucleic acids, such as genes. For example, the homology can be calculated after aligning the two sequences so that the homology is at its highest level.

Another way of calculating homology can be performed by published algorithms. Optimal alignment of sequences for comparison may be conducted by the local homology algorithm of Smith and Waterman Adv. Appl. Math. 2: 482 (1981), by the homology alignment algorithm of Needleman and Wunsch, J. Mol. Biol. 48: 443 (1970), by the search for similarity method of Pearson and Lipman, Proc. Natl. Acad. Sci. U.S.A. 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by inspection.

The same types of homology can be obtained for nucleic acids by for example the algorithms disclosed in Zuker, M. Science 244:48-52, 1989, Jaeger et al. Proc. Natl. Acad. Sci. USA 86:7706-7710, 1989, Jaeger et al. Methods Enzymol. 183:281-306, 1989 which are herein incorporated by reference for at least material related to nucleic acid alignment. It is understood that any of the methods typically can be used and that in certain instances the results of these various methods may differ, but the skilled artisan understands if identity is found with at least one of these methods, the sequences would be said to have the stated identity, and be disclosed herein.

For example, as used herein, a sequence recited as having a particular percent homology to another sequence refers to sequences that have the recited homology as calculated by any one or more of the calculation methods described above. For example, a first sequence has 80 percent homology, as defined herein, to a second sequence if the first sequence is calculated to have 80 percent homology to the second sequence using the Zuker calculation method even if the first sequence does not have 80 percent homology to the second sequence as calculated by any of the other calculation methods. As another example, a first sequence has 80 percent homology, as defined herein, to a second sequence if the first sequence is calculated to have 80 percent homology to the second sequence using both the Zuker calculation method and the Pearson and Lipman calculation method even if the first sequence does not have 80 percent homology to the second sequence as calculated by the Smith and Waterman calculation method, the Needleman and Wunsch calculation method, the Jaeger calculation methods, or any of the other calculation methods. As yet another example, a first sequence has 80 percent homology, as defined herein, to a second sequence if the first sequence is calculated to have 80 percent homology to the second sequence using each of calculation methods (although, in practice, the different calculation methods will often result in different calculated homology percentages).

12. Hybridization/Selective Hybridization

The term hybridization typically means a sequence driven interaction between at least two nucleic acid molecules, such as a primer or a probe and a gene. Sequence driven interaction means an interaction that occurs between two nucleotides or nucleotide analogs or nucleotide derivatives in a nucleotide specific manner. For example, G interacting with C or A interacting with T are sequence driven interactions. Typically sequence driven interactions occur on the Watson-Crick face or Hoogsteen face of the nucleotide. The hybridization of two nucleic acids is affected by a number of conditions and parameters known to those of skill in the art. For example, the salt concentrations, pH, and temperature of the reaction all affect whether two nucleic acid molecules will hybridize.

Parameters for selective hybridization between two nucleic acid molecules are well known to those of skill in the art. For example, selective hybridization conditions can be defined as stringent hybridization conditions. For example, stringency of hybridization is controlled by both temperature and salt concentration of either or both of the hybridization and washing steps. For example, the conditions of hybridization to achieve selective hybridization may involve hybridization in high ionic strength solution (6×SSC or 6×SSPE) at a temperature that is about 12-25° C. below the Tm (the melting temperature at which half of the molecules dissociate from their hybridization partners) followed by washing at a combination of temperature and salt concentration chosen so that the washing temperature is about 5° C. to 20° C. below the Tm. The temperature and salt conditions are readily determined empirically in preliminary experiments in which samples of reference DNA immobilized on filters are hybridized to a labeled nucleic acid of interest and then washed under conditions of different stringencies. Hybridization temperatures are typically higher for DNA-RNA and RNA-RNA hybridizations. The conditions can be used as described above to achieve stringency, or as is known in the art. (Sambrook et al., Molecular Cloning: A Laboratory Manual, 2nd Ed., Cold Spring Harbor Laboratory, Cold Spring Harbor, N.Y., 1989; Kunkel et al. Methods Enzymol. 1987:154:367, 1987 which is herein incorporated by reference for material at least related to hybridization of nucleic acids). A preferable stringent hybridization condition for a DNA:DNA hybridization can be at about 68° C. (in aqueous solution) in 6×SSC or 6×SSPE followed by washing at 68° C. Stringency of hybridization and washing, if desired, can be reduced accordingly as the degree of complementarity desired is decreased, and further, depending upon the G-C or A-T richness of any area wherein variability is searched for. Likewise, stringency of hybridization and washing, if desired, can be increased accordingly as homology desired is increased, and further, depending upon the G-C or A-T richness of any area wherein high homology is desired, all as known in the art.

Another way to define selective hybridization is by looking at the amount (percentage) of one of the nucleic acids bound to the other nucleic acid. For example, selective hybridization conditions can be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the limiting nucleic acid is bound to the non-limiting nucleic acid. Typically, the non-limiting primer is in for example, 10 or 100 or 1000 fold excess. This type of assay can be performed at under conditions where both the limiting and non-limiting primer are for example, 10 fold or 100 fold or 1000 fold below their k_{d}, or where only one of the nucleic acid molecules is 10 fold or 100 fold or 1000 fold or where one or both nucleic acid molecules are above their k_{d}.

Another way to define selective hybridization is by looking at the percentage of primer that gets enzymatically manipulated under conditions where hybridization is required to promote the desired enzymatic manipulation. For example, selective hybridization conditions can be when at least about, 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer is enzymatically manipulated under conditions which promote the enzymatic manipulation, for example if the enzymatic manipulation is DNA extension, then selective hybridization conditions would be when at least about 60, 65, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100 percent of the primer molecules are extended. Conditions can also include those suggested by the manufacturer or indicated in the art as being appropriate for the enzyme performing the manipulation.

Just as with homology, it is understood that there are a variety of methods herein disclosed for determining the level of hybridization between two nucleic acid molecules. It is understood that these methods and conditions may provide different percentages of hybridization between two nucleic acid molecules, but unless otherwise indicated meeting the parameters of any of the methods would be sufficient. For example if 80% hybridization was required and as long as hybridization occurs within the required parameters in any one of these methods it is considered disclosed herein.

It is understood that those of skill in the art understand that if a composition or method meets any one of these criteria for determining hybridization either collectively or singly it is a composition or method that is disclosed herein.

### B. METHODS OF USING THE COMPOSITIONS

The disclosed compositions can be used in a variety of ways as research tools. Other uses are disclosed, apparent from the disclosure, and/or will be understood by those in the art.

For example, provided is a method of screening for an agent for use in treating or preventing synovial sarcoma, comprising administering a candidate agent to a non-human animal disclosed herein and monitoring the animal for synovial sarcoma development or progression.

In general, candidate agents can be identified from large libraries of natural products or synthetic (or semi-synthetic) extracts or chemical libraries according to methods known in the art. Those skilled in the field of drug discovery and development will understand that the precise source of test extracts or compounds is not critical to the screening procedure(s) of the invention. Accordingly, virtually any number of chemical extracts or compounds can be screened using the exemplary methods described herein. Examples of such extracts or compounds include, but are not limited to, plant-, fungal-, prokaryotic- or animal-based extracts, fermentation broths, and synthetic compounds, as well as modification of existing compounds. Numerous methods are also available for generating random or directed synthesis (e.g., semi-synthesis or total synthesis) of any number of chemical compounds, including, but not limited to, saccharide-, lipid-, peptide-, polypeptide- and nucleic acid-based compounds. Synthetic compound libraries are commercially available, e.g., from Brandon Associates (Merrimack, N.H.) and Aldrich Chemical (Milwaukee, Wis.). Alternatively, libraries of natural compounds in the form of bacterial, fungal, plant, and animal extracts are commercially available from a number of sources, including Biotics (Sussex, UK), Xenova (Slough, UK), Harbor Branch Oceangraphics Institute (Ft. Pierce, Fla.), and PharmaMar, U.S.A. (Cambridge, Mass.). In addition, natural and synthetically produced libraries are produced, if desired, according to methods known in the art, e.g., by standard extraction and fractionation methods. Furthermore, if desired, any library or compound is readily modified using standard chemical, physical, or biochemical methods. In addition, those skilled in the art of drug discovery and development readily understand that methods for dereplication (e.g., taxonomic dereplication, biological dereplication, and chemical dereplication, or any combination thereof) or the elimination of replicates or repeats of materials already known for their effect on the activity of synovival sarcoma should be employed whenever possible.

When a crude extract is found to have a desired activity, further fractionation of the positive lead extract is necessary to isolate chemical constituents responsible for the observed effect. Thus, the goal of the extraction, fractionation, and purification process is the careful characterization and identification of a chemical entity within the crude extract having an activity that inhibits synovival sarcoma. The same assays described herein for the detection of activities in mixtures of compounds can be used to purify the active component and to test derivatives thereof. Methods of fractionation and purification of such heterogenous extracts are known in the art. If desired, compounds shown to be useful agents for treatment are chemically modified according to methods known in the art.

Candidate agents encompass numerous chemical classes, but are most often organic molecules, e.g., small organic compounds having a molecular weight of more than 100 and less than about 2,500 daltons. Candidate agents comprise functional groups necessary for structural interaction with proteins, particularly hydrogen bonding, and typically include at least an amine, carbonyl, hydroxyl or carboxyl group, for example, at least two of the functional chemical groups. The candidate agents often comprise cyclical carbon or heterocyclic structures and/or aromatic or polyaromatic structures substituted with one or more of the above functional groups. Candidate agents are also found among biomolecules including peptides, saccharides, fatty acids, steroids, purines, pyrimidines, derivatives, structural analogs or combinations thereof. In a further embodiment, candidate agents are peptides.

In some embodiments, the candidate agents are proteins. In some aspects, the candidate agents are naturally occurring proteins or fragments of naturally occurring proteins. Thus, for example, cellular extracts containing proteins, or random or directed digests of proteinaceous cellular extracts, can be used. In this way libraries of procaryotic and eucaryotic proteins can be made for screening using the methods herein. The libraries can be bacterial, fungal, viral, and vertebrate proteins, and human proteins.

### C. METHODS OF MAKING THE COMPOSITIONS

The compositions disclosed herein and the compositions necessary to perform the disclosed methods can be made using any method known to those of skill in the art for that particular reagent or compound unless otherwise specifically noted.

Also provided herein is a method of producing a non-human transgenic animal comprising introducing a nucleotide sequence encoding SYT-SSX operably linked to an expression control sequence into a fertilized animal oocyte; allowing the fertilized animal oocyte to develop to term; and identifying a transgenic animal whose genome comprises the SYT-SSX nucleotide sequence, wherein expression of the SYT-SSX results in synovial sarcoma in the animal. Also provided herein is a method for producing a non-human transgenic animal comprising providing a vector comprising a nucleotide sequence encoding SYT-SSX operably linked to an expression control sequence; introducing the expression vector into a fertilized animal oocyte; allowing said fertilized animal oocyte to develop to term; and identifying a transgenic animal whose genome comprises the SYT-SSX nucleotide sequence, wherein expression of said SYT-SSX results in synovial sarcoma in the animal.

Also provided herein is a method comprising administering a vector comprising a nucleotide sequence encoding SYT-SSX operably linked to an expression control sequence to an animal, wherein expression of said SYT-SSX results in synovial sarcoma in the animal.

1. Transgenic Mice Models

i. Methods of Producing Transgenic Animals

The nucleic acids and vectors provided herein can be used to produce transgenic animals. Various methods are known for producing a transgenic animal. In one method, an embryo at the pronuclear stage (a “one cell embryo”) is harvested from a female and the transgene is microinjected into the embryo, in which case the transgene will be chromosomally integrated into the germ cells and somatic cells of the resulting mature animal. In another method, embryonic stem cells are isolated and the transgene is incorporated into the stem cells by electroporation, plasmid transfection or microinjection; the stem cells are then reintroduced into the embryo, where they colonize and contribute to the germ line. Methods for microinjection of polynucleotides into mammalian species are described, for example, in U.S. Pat. No. 4,873,191, which is incorporated herein by reference. In yet another method, embryonic cells are infected with a retrovirus containing the transgene, whereby the germ cells of the embryo have the transgene chromosomally integrated therein. When the animals to be made transgenic are avian, microinjection into the pronucleus of the fertilized egg is problematic because avian fertilized ova generally go through cell division for the first twenty hours in the oviduct and, therefore, the pronucleus is inaccessible. Thus, the retrovirus infection method can be used for making transgenic avian species (see U.S. Pat. No. 5,162,215, which is incorporated herein by reference). If microinjection is to be used with avian species, however, the embryo can be obtained from a sacrificed hen approximately 2.5 hours after the laying of the previous laid egg, the transgene is microinjected into the cytoplasm of the germinal disc and the embryo is cultured in a host shell until maturity (Love et al., Biotechnology 12, 1994). When the animals to be made transgenic are bovine or porcine, microinjection can be hampered by the opacity of the ova, thereby making the nuclei difficult to identify by traditional differential interference-contrast microscopy. To overcome this problem, the ova first can be centrifuged to segregate the pronuclei for better visualization.

The transgene can be introduced into embryonal target cells at various developmental stages, and different methods are selected depending on the stage of development of the embryonal target cell. The zygote is the best target for microinjection. The use of zygotes as a target for gene transfer has a major advantage in that the injected DNA can incorporate into the host gene before the first cleavage (Brinster et al., Proc. Natl. Acad. Sci., USA 82:4438-4442, 1985). As a consequence, all cells of the transgenic non-human animal carry the incorporated transgene, thus contributing to efficient transmission of the transgene to offspring of the founder, since 50% of the germ cells will harbor the transgene.

A transgenic animal can be produced by crossbreeding two chimeric animals, each of which includes exogenous genetic material within cells used in reproduction. Twenty-five percent of the resulting offspring will be transgenic animals that are homozygous for the exogenous genetic material, 50% of the resulting animals will be heterozygous, and the remaining 25% will lack the exogenous genetic material and have a wild type phenotype.

In the microinjection method, the transgene is digested and purified free from any vector DNA, for example, by gel electrophoresis. The transgene can include an operatively associated promoter, which interacts with cellular proteins involved in transcription, and provides for constitutive expression, tissue specific expression, developmental stage specific expression, or the like. Such promoters include those from cytomegalovirus (CMV), Moloney leukemia virus (MLV), and herpes virus, as well as those from the genes encoding metallothionein, skeletal actin, Phosphenolpyruvate carboxylase (PEPCK), phosphoglycerate (PGK), dihydrofolate reductase (DHFR), and thymidine kinase (TK). Promoters from viral long terminal repeats (LTRs) such as Rous sarcoma virus LTR also can be employed. When the animals to be made transgenic are avian, promoters include those for the chicken [bgr]-globin gene, chicken lysozyme gene, and avian leukosis virus. Constructs useful in plasmid transfection of embryonic stem cells will employ additional regulatory elements, including, for example, enhancer elements to stimulate transcription, splice acceptors, termination and polyadenylation signals, ribosome binding sites to permit translation, and the like.

In the retroviral infection method, the developing non-human embryo can be cultured in vitro to the blastocyst stage. During this time, the blastomeres can be targets for retroviral infection (Jaenich, Proc. Natl. Acad. Sci. USA 73:1260-1264, 1976). Efficient infection of the blastomeres is obtained by enzymatic treatment to remove the zona pellucida (Hogan et al., Manipulating the Mouse Embryo (Cold Spring Harbor Laboratory Press, 1986). The viral vector system used to introduce the transgene is typically a replication-defective retrovirus carrying the transgene (Jahner et al., Proc. Natl. Acad. Sci., USA 82:6927-6931, 1985; Van der Putten et al., Proc. Natl. Acad. Sci. USA 82:6148-6152, 1985). Transfection is easily and efficiently obtained by culturing the blastomeres on a monolayer of virus producing cells (Van der Putten et al., supra, 1985; Stewart et al., EMBO J. 6:383-388, 1987). Alternatively, infection can be performed at a later stage. Virus or virus-producing cells can be injected into the blastocoele (Jahner et al., Nature 298:623-628, 1982). Most of the founders will be mosaic for the transgene since incorporation occurs only in a subset of the cells which formed the transgenic nonhuman animal. Further, the founder can contain various retroviral insertions of the transgene at different positions in the genome, which generally will segregate in the offspring. In addition, it is also possible to introduce transgenes into the germ line, albeit with low efficiency, by intrauterine retroviral infection of the mid-gestation embryo (Jahner et al., supra, 1982).

Embryonal stem cell (ES) also can be targeted for introduction of the transgene. ES cells are obtained from pre-implantation embryos cultured in vitro and fused with embryos (Evans et al. Nature 292:154-156, 1981; Bradley et al., Nature 309:255-258, 1984; Gossler et al., Proc. Natl. Acad. Sci., USA 83:9065-9069, 1986; Robertson et al., Nature 322:445-448, 1986). Transgenes can be efficiently introduced into the ES cells by DNA transfection or by retrovirus mediated transduction. Such transformed ES cells can thereafter be combined with blastocysts from a nonhuman animal. The ES cells thereafter colonize the embryo and contribute to the germ line of the resulting chimeric animal (see Jaenisch, Science 240:1468-1474, 1988).

“Founder” generally refers to a first transgenic animal, which has been obtained from any of a variety of methods, e.g., pronuclei injection. An “inbred animal line” is intended to refer to animals which are genetically identical at all endogenous loci.

ii. Crosses

It is understood that the animals provided herein can be crossed with other animals. For example, wherein the provided animals are mice, they can be crossed with mice expressing Cre recombinase in cells or tissues that can be a source of synovial sarcoma. Such mice could be those expressing Cre in, for example, cartilage, bone, and/or fibroblasts. One can also cross the disclosed mouse model to mice that harbor mutation in genes commonly disregulated/misexpressed/mutated in human synovial sarcomas such as, for example, p53 and/or Ink4, to investigate the effects of these second genetic hits on tumor progression or tumor prognosis.

2. Nucleic Acid Synthesis

For example, the nucleic acids, such as, the oligonucleotides to be used as primers can be made using standard chemical synthesis methods or can be produced using enzymatic methods or any other known method. Such methods can range from standard enzymatic digestion followed by nucleotide fragment isolation (see for example, Sambrook et al., Molecular Cloning: A Laboratory Manual, 2nd Edition (Cold Spring Harbor Laboratory Press, Cold Spring Harbor, N.Y., 1989) Chapters 5, 6) to purely synthetic methods, for example, by the cyanoethyl phosphoramidite method using a Milligen or Beckman System 1Plus DNA synthesizer (for example, Model 8700 automated synthesizer of Milligen-Biosearch, Burlington, Mass. or ABI Model 380B). Synthetic methods useful for making oligonucleotides are also described by Ikuta et al., Ann. Rev. Biochem. 53:323-356 (1984), (phosphotriester and phosphite-triester methods), and Narang et al., Methods Enzymol., 65:610-620 (1980), (phosphotriester method). Protein nucleic acid molecules can be made using known methods such as those described by Nielsen et al., Bioconjug. Chem. 5:3-7 (1994).

3. Peptide Synthesis

One method of producing the disclosed proteins, such as SEQ ID NO:2, is to link two or more peptides or polypeptides together by protein chemistry techniques. For example, peptides or polypeptides can be chemically synthesized using currently available laboratory equipment using either Fmoc (9-fluorenylmethyloxycarbonyl) or Boc (tert-butyloxycarbonoyl) chemistry. (Applied Biosystems, Inc., Foster City, Calif.). One skilled in the art can readily appreciate that a peptide or polypeptide corresponding to the disclosed proteins, for example, can be synthesized by standard chemical reactions. For example, a peptide or polypeptide can be synthesized and not cleaved from its synthesis resin whereas the other fragment of a peptide or protein can be synthesized and subsequently cleaved from the resin, thereby exposing a terminal group which is functionally blocked on the other fragment. By peptide condensation reactions, these two fragments can be covalently joined via a peptide bond at their carboxyl and amino termini, respectively, to form an antibody, or fragment thereof. (Grant GA (1992) Synthetic Peptides: A User Guide. W.H. Freeman and Co., N.Y. (1992); Bodansky M and Trost B., Ed. (1993) Principles of Peptide Synthesis. Springer-Verlag Inc., NY (which is herein incorporated by reference at least for material related to peptide synthesis). Alternatively, the peptide or polypeptide is independently synthesized in vivo as described herein. Once isolated, these independent peptides or polypeptides may be linked to form a peptide or fragment thereof via similar peptide condensation reactions.

For example, enzymatic ligation of cloned or synthetic peptide segments allow relatively short peptide fragments to be joined to produce larger peptide fragments, polypeptides or whole protein domains (Abrahmsen L et al., Biochemistry, 30:4151 (1991)). Alternatively, native chemical ligation of synthetic peptides can be utilized to synthetically construct large peptides or polypeptides from shorter peptide fragments. This method consists of a two step chemical reaction (Dawson et al. Synthesis of Proteins by Native Chemical Ligation. Science, 266:776-779 (1994)). The first step is the chemoselective reaction of an unprotected synthetic peptide—thioester with another unprotected peptide segment containing an amino-terminal Cys residue to give a thioester-linked intermediate as the initial covalent product. Without a change in the reaction conditions, this intermediate undergoes spontaneous, rapid intramolecular reaction to form a native peptide bond at the ligation site (Baggiolini M et al. (1992) FEBS Lett. 307:97-101; Clark-Lewis I et al., J. Biol. Chem., 269:16075 (1994); Clark-Lewis I et al., Biochemistry, 30:3128 (1991); Rajarathnam K et al., Biochemistry 33:6623-30 (1994)).

Alternatively, unprotected peptide segments are chemically linked where the bond formed between the peptide segments as a result of the chemical ligation is an unnatural (non-peptide) bond (Schnolzer, M et al. Science, 256:221 (1992)). This technique has been used to synthesize analogs of protein domains as well as large amounts of relatively pure proteins with full biological activity (deLisle Milton R C et al., Techniques in Protein Chemistry IV. Academic Press, New York, pp. 257-267 (1992)).

4. Process Claims for Making the Compositions

Disclosed are processes for making the compositions as well as making the intermediates leading to the compositions. There are a variety of methods that can be used for making these compositions, such as synthetic chemical methods and standard molecular biology methods. It is understood that the methods of making these and the other disclosed compositions are specifically disclosed.

Disclosed are nucleic acid molecules produced by the process comprising linking in an operative way a nucleic acid comprising the sequence set forth in SEQ ID NO:1 and a sequence controlling the expression of the nucleic acid.

Also disclosed are nucleic acid molecules produced by the process comprising linking in an operative way a nucleic acid molecule comprising a sequence having 80% identity to a sequence set forth in SEQ ID NO:1, and a sequence controlling the expression of the nucleic acid.

Disclosed are nucleic acid molecules produced by the process comprising linking in an operative way a nucleic acid molecule comprising a sequence that hybridizes under stringent hybridization conditions to a sequence set forth SEQ ID NO:1 and a sequence controlling the expression of the nucleic acid.

Disclosed are nucleic acid molecules produced by the process comprising linking in an operative way a nucleic acid molecule comprising a sequence encoding a peptide set forth in SEQ ID NO:2 and a sequence controlling an expression of the nucleic acid molecule.

Disclosed are nucleic acid molecules produced by the process comprising linking in an operative way a nucleic acid molecule comprising a sequence encoding a peptide having 80% identity to a peptide set forth in SEQ ID NO:3 and a sequence controlling an expression of the nucleic acid molecule.

Disclosed are nucleic acids produced by the process comprising linking in an operative way a nucleic acid molecule comprising a sequence encoding a peptide having 80% identity to a peptide set forth in SEQ ID NO:2, wherein any change is a conservative changes and a sequence controlling an expression of the nucleic acid molecule.

Disclosed are cells produced by the process of transforming the cell with any of the disclosed nucleic acids. Disclosed are cells produced by the process of transforming the cell with any of the non-naturally occurring disclosed nucleic acids.

Disclosed are any of the disclosed peptides produced by the process of expressing any of the disclosed nucleic acids. Disclosed are any of the non-naturally occurring disclosed peptides produced by the process of expressing any of the disclosed nucleic acids. Disclosed are any of the disclosed peptides produced by the process of expressing any of the non-naturally disclosed nucleic acids.

Disclosed are animals produced by the process of transfecting a cell within the animal with any of the nucleic acid molecules disclosed herein. Disclosed are animals produced by the process of transfecting a cell within the animal any of the nucleic acid molecules disclosed herein, wherein the animal is a mammal. Also disclosed are animals produced by the process of transfecting a cell within the animal any of the nucleic acid molecules disclosed herein, wherein the mammal is mouse, rat, rabbit, cow, sheep, pig, or primate.

Also disclose are animals produced by the process of adding to the animal any of the cells disclosed herein.

### D. DEFINITIONS

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents.

It must be noted that as used herein and in the appended claims, the singular forms “a,” “an,” and “the” include plural reference unless the context clearly dictates otherwise. Thus, for example, reference to “a peptide” includes a plurality of such peptides, reference to “the peptide” is a reference to one or more peptides and equivalents thereof known to those skilled in the art, and so forth.

“Optional” or “optionally” means that the subsequently described event, circumstance, or material may or may not occur or be present, and that the description includes instances where the event, circumstance, or material occurs or is present and instances where it does not occur or is not present.

Ranges can be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that when a value is disclosed that “less than or equal to” the value, “greater than or equal to the value” and possible ranges between values are also disclosed, as appropriately understood by the skilled artisan. For example, if the value “10” is disclosed the “less than or equal to 10” as well as “greater than or equal to 10” is also disclosed. It is also understood that the throughout the application, data is provided in a number of different formats, and that this data, represents endpoints and starting points, and ranges for any combination of the data points. For example, if a particular data point “10” and a particular data point 15 are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Throughout the description and claims of this specification, the word “comprise” and variations of the word, such as “comprising” and “comprises,” means “including but not limited to,” and is not intended to exclude, for example, other additives, components, integers or steps.

Throughout this application, various publications are referenced. The disclosures of these publications in their entireties are hereby incorporated by reference into this application in order to more fully describe the state of the art to which this pertains. The references disclosed are also individually and specifically incorporated by reference herein for the material contained in them that is discussed in the sentence in which the reference is relied upon.

### E. EXAMPLES

The following examples are put forth so as to provide those of ordinary skill in the art with a complete disclosure and description of how the compounds, compositions, articles, devices and/or methods claimed herein are made and evaluated, and are intended to be purely exemplary and are not intended to limit the disclosure. Efforts have been made to ensure accuracy with respect to numbers (e.g., amounts, temperature, etc.), but some errors and deviations should be accounted for. Unless indicated otherwise, parts are parts by weight, temperature is in ° C. or is at ambient temperature, and pressure is at or near atmospheric.

### 1. Example 1

### i. Results

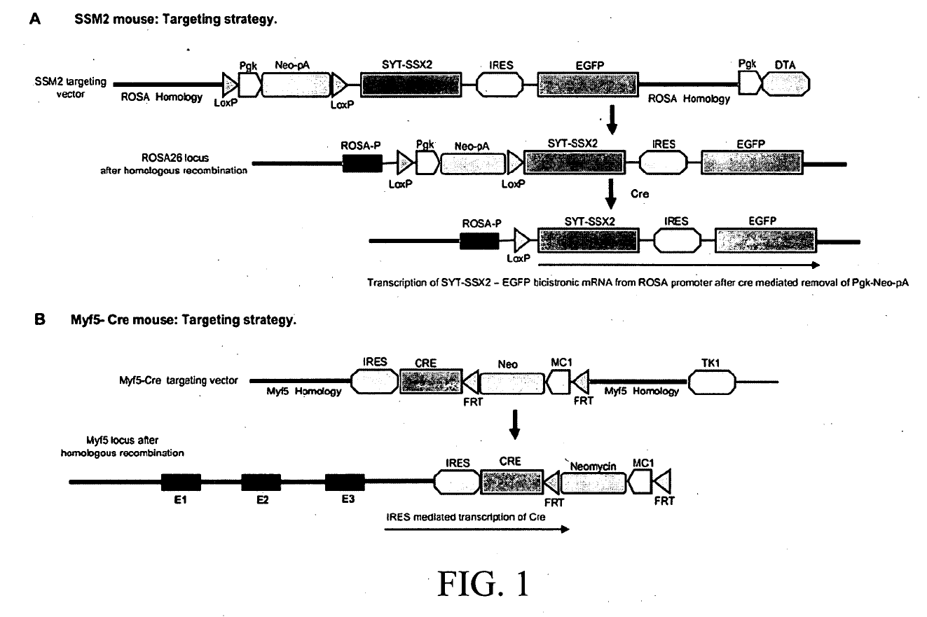

Generation of Targeted Mouse Lines: Human SYT-SSX2 cDNA generated from RNA isolated from a synovial sarcoma tumor sample was used to construct conditional SYT-SSX2 targeting vectors for targeting into the mouse ROSA26 locus on chromosome 6 following published procedures (Srinivas et al., 2001). The ROSA promoter is ubiquitously active, thereby allowing transcription of the fusion protein in any chosen tissue following Cre-dependent recombination (Soriano, 1999; Zambrowicz et al., 1997). Two variations of the targeting vector were designed and used to generate two mouse lines, SSM1 (synovial sarcoma mouse 1) and SSM2 (synovial sarcoma mouse 2). SSM2 mice express SYT-SSX2-IRES-EGFP bicistronic mRNA from the endogenous ROSA promoter that enables monitoring of SYT-SSX2 expression by detecting EGFP-mediated fluorescence (FIG. 1A). The SSM1-targeting construct is similar but lacks the IRES-EGFP marker gene. Between the ROSA26 promoter and the SYT-SSX2 cDNA is a strong transcriptional termination signal, NeoPA, flanked by loxP sites (FIG. 1A). In the absence of Cre, SYT-SSX2 is not transcribed. In the presence of Cre, Neo-pA is excised and SYT-SSX2 transcription commences. By controlling where and when Cre protein is produced, where and when SYT-SSX2 fusion protein is made is controled. In the absence of Cre, both homozygous and heterozygous SSM1 and SSM2 mice (SSM denoting either mouse lines) are normal, viable, and fertile with no expression of SYT-SSX2 or EGFP.

To induce Cre expression within committed myoblasts expressing the myogenic regulatory factor Myf5, a Myf5-Cre driver was generated such that Cre is expressed as a second cistron from an IRES placed within the 3′UTR of the Myf5 gene as shown in FIG. 1B (Jackson et al., 1990; Jang and Wimmer, 1990). Homozygous and heterozygous Myf5-Cre mice were viable and fertile. To confirm the correct expression domain of Cre recombinase in Myf5-Cre mice, they were bred to ROSA-YFP reporter mice that express the yellow fluorescent protein (YFP) within any Cre-expressing cell and its lineage (Srinivas et al., 2001). Myf5 is a myogenic regulatory factor that has an important role in the specification of skeletal muscle lineage. Its expression begins early in embryogenesis within immature myoblasts that eventually give rise to adult skeletal muscle (Chanoine et al., 2004; Pownall et al., 2002). Therefore, while early-stage Myf5-Cre/ROSA-YFP embryos should have YFP expression within dermomyotome component of somites, later-stage embryos and adults should have YFP expression within skeletal muscle. This was consistently observed in the disclosed Myf5 lineage experiments (FIGS. 2Aa-2Ad).

SYT-SSX2 Expression within Myf5 Lineage Induces Tumors: To express SYT-SSX2 within committed myoblasts and their lineage, the conditional SSM (SSM1 or SSM2) mice were bred to Myf5-Cre mice, and the resulting Myf5-Cre/SSM progenies were followed. About 8% of these mice were born significantly smaller than their siblings and usually died by 2 months of age. However, 100% of the surviving Myf5-Cre/SSM mice (18/18) developed tumors between the ages of 3-5 months, demonstrating complete penetrance in terms of tumor induction. Control littermates (>100) that included mice harboring only the SSM or Myf5-Cre alleles were followed for more than a year with no tumor induction or any other abnormalities. The Myf5-Cre/SSM mice that were born significantly smaller and died before 2 months did not harbor any apparent tumors and died of tumor-unrelated causes.

Multiple tumors (three to five per mouse) were detected within Myf5-Cre/SSM mice upon necropsy. Detection of very small tumors as well as potential metastasis was aided by expression of the EGFP marker protein incorporated in our design of SSM2 mice. All tumors detected within Myf5-Cre/SSM2 mice had intense green fluorescence, characteristic of EGFP expression (FIGS. 2Bc and 2Be). Expression of SYT-SSX2 was also confirmed by RTPCR on total RNA from tumors. Most tumors were located within skeletal muscle, a predicted outcome since the fusion protein was induced within the skeletal-muscle-specific Myf5 lineage (Table 3). However, a minority of small EGFP-positive tumors were observed in nonskeletal muscle tissue, such as the cerebellum (which is neither derived nor in close proximity to Myf5-derived tissue) indicative of potential metastasis (FIG. 2Bf).

Histological and Immunohistochemical Profiles of Mouse Tumors Recapitulate Human Synovial Sarcoma: On macroscopic examination, the mouse tumors were vascularized, often having a chalky white appearance distinct from the surrounding tissue (FIG. 2Bb, arrows). Most frequently affected were the musculatures of the limb near joints and the intercostal region (FIGS. 2Ba, 2Bb, and 2Bd and Table 3). The tumors were often hemorrhagic (FIG. 3Ac, white arrow), with cystic spaces often detected within larger tumors (FIG. 3Ac, black arrow). Histology revealed striking similarity to human synovial sarcoma, with both biphasic (FIGS. 3Aa-3Ac) and monophasic variants (FIG. 3Ad) identified. However, the monophasic variants greatly outnumbered biphasic (13 monophasic and 3 biphasic). This correlates well with data from human cases showing correlation of SYT-SSX2 with the monophasic subtype (Ladanyi et al., 2002). While smaller tumors were usually monophasic, often containing trapped skeletal muscle fibers (FIG. 3Ad, black arrow), the larger tumors usually showed biphasic histology with epithelioid cells arranged in a glandular pattern amid spindle cells (FIG. 3Ab, black arrow). Myxoid changes detected by alcian blue staining (FIG. 3Ae) and fibrous changes detected by Masson's trichrome staining (FIG. 3Af) also recapitulatde features of human synovial sarcoma.

Synovial sarcomas coexpress epithelial as well as mesenchymal markers and often overexpress Bcl-2. This is faithfully recapitulated in tumors generated in the disclosed mouse model that show expression of epithelial cytokeratins (positive for cytokeratin AE1/AE2 cocktail and CAM5.2) as well as mesenchymal marker vimentin (FIGS. 3Ba-3Bd). In humans, synovial sarcoma is considered a high-grade tumor, which is recapitulated in the disclosed model based on widespread expression of the proliferation marker Mib (FIG. 3Be). Since the tumors were induced within skeletal muscle lineage, immunohistochemistry for myogenin was done to rule out other muscle tumors, such as rhabdomyosarcomas, that are usually positive for myogenin (while synovial sarcomas are usually myogenin negative). The tumors generated in the disclosed model were negative for myogenin (FIG. 3Bf). In summary, based on histopathology and immunohistochemistry, the mouse tumors strongly resemble humansynovial sarcoma.

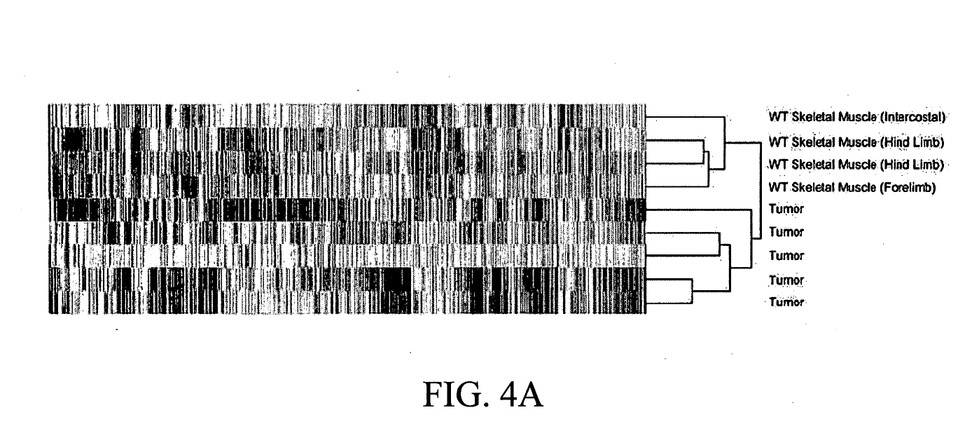

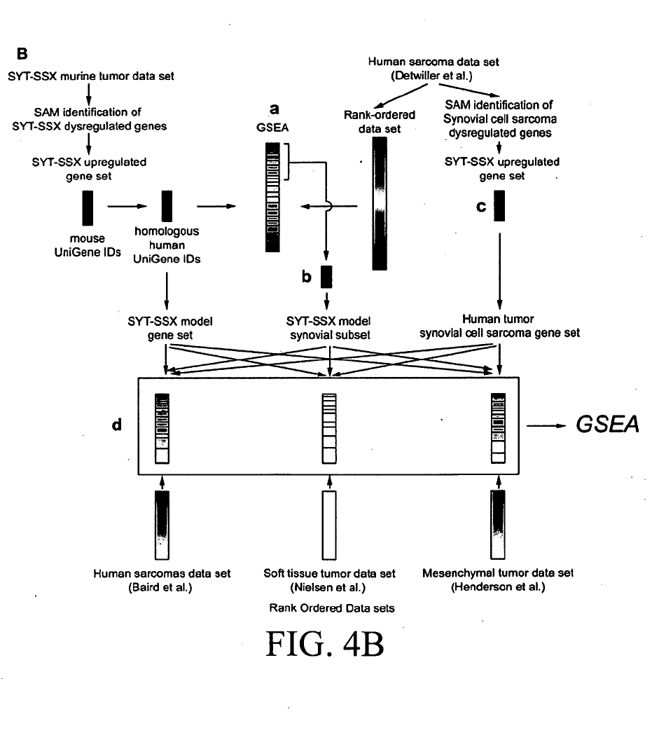

Transcriptional Profiles of the Mouse Tumors Recapitulate Human Synovial Sarcoma Profiles: To further compare the murine tumors to their human counterparts, transcriptional profiling analysis using Affymetrix mouse genome 430 2.0 gene chip was performed on five independent tumors and compared to four skeletal muscle samples from wild-type control mice. Hierarchical clustering of preprocessed and normalized expression profiles showed segregation of tumors and normal muscle samples (FIG. 4A). Subsequent significance analysis of microarrays (SAM) identified 1736 upregulated and 2341 downregulated genes at a false discovery rate (FDR) of <0.01. To determine if the expression profile of these mouse tumors simulated human synovial sarcoma, the disclosed gene expression pattern was compared to synovial sarcoma expression profiles present within several published human tumor expression data sets (Baird et al., 2005; Detwiller et al., 2005; Henderson et al., 2005; Nielsen et al., 2002). The genes were first rank-ordered in each data set according to their correlation to synovial sarcoma and a list of significantly correlating genes extracted as determined by permutation testing at a p value of <0.01 (Golub et al., 1999) extracted. The murine tumor data were similarly processed and converted to homologous human genes. The murine and human rank-ordered lists were compared via Spearman correlation testing. The results of the Spearman correlation showed small but significant similarities between murine SYT-SSX2-induced tumors and human synovial sarcomas (Table 4). Thus, the murine tumors were indeed synovial sarcomas.

It was reasoned that the small correlations observed in the Spearman analysis were due to a previously unidentified SYT-SSX gene expression signature and that one could identify this signature by comparisons between the murine and human tumor data sets. To test this hypothesis, a more sensitive statistical method, gene set enrichment analysis (GSEA), was employed. GSEA offers a straightforward means of measuring the “enrichment” of one gene set against an ordered data set in which genes are ranked according to their correlation to the phenotype of interest. GSEA has been used previously to compare data sets derived from different microarray platforms and from different species (Sweet-Cordero et al., 2005). An “SYT-SSX model gene set” was first derived from the murine tumor data and the murine genes mapped to their human counterparts. An initial GSEA comparison between this murine gene set and a human synovial sarcoma data set (Detwiller et al., 2005) did not demonstrate a statistically significant enrichment (ES 0.2, p=0.48). This lack of enrichment does not necessarily indicate a strong dissimilarity between the murine model and the human disease but instead could be due to the mode of selection of the model signature or difficulties in comparing data sets across species or platforms. This initial comparison was therefore used as a “training set” to derive a common signature between the murine model and the Detwiller data set. 55 genes were identified that were enriched between the murine tumors and the training data set and this list was named “SYT-SSX model synovial subset.” Importantly, this gene set was largely distinct from a set of 249 synovial-sarcoma-specific genes derived from the Detwiller data set using SAM (only four genes overlapped between these two gene sets). To determine if this “SYTSSX model synovial subset” of genes truly represented a new group of synovial sarcoma genes, the performance of this gene set was compared with the performance of a SAM-derived Detwiller synovial sarcoma gene set (designated as “human tumor synovial cell sarcoma gene set”) using GSEA against three unique sarcoma “test data sets” (Baird, Nielsen, and Henderson data sets). The entire process is schematically represented in FIG. 4B. The “SYT-SSX model synovial subset” is significantly represented in only human synovial sarcomas and not in other similar human sarcomas across all human tumor data sets (Table 2). This demonstrates that comparisons between the murine and human tumor data sets can reveal synovial sarcoma-specific genes that could not otherwise be detected and further highlights the similarity between our murine tumors and human synovial sarcoma.

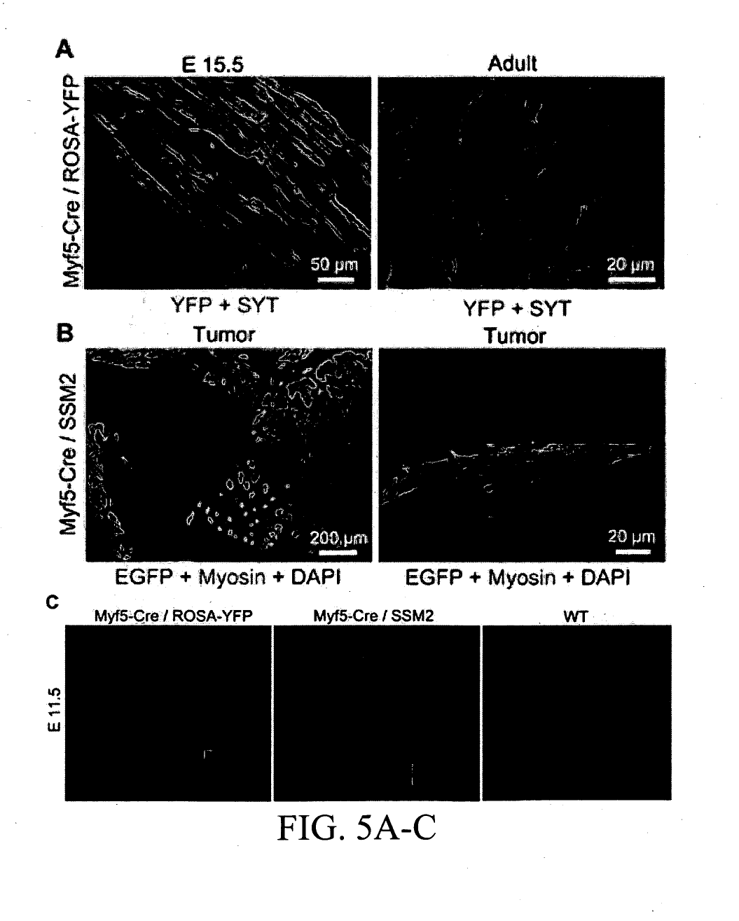

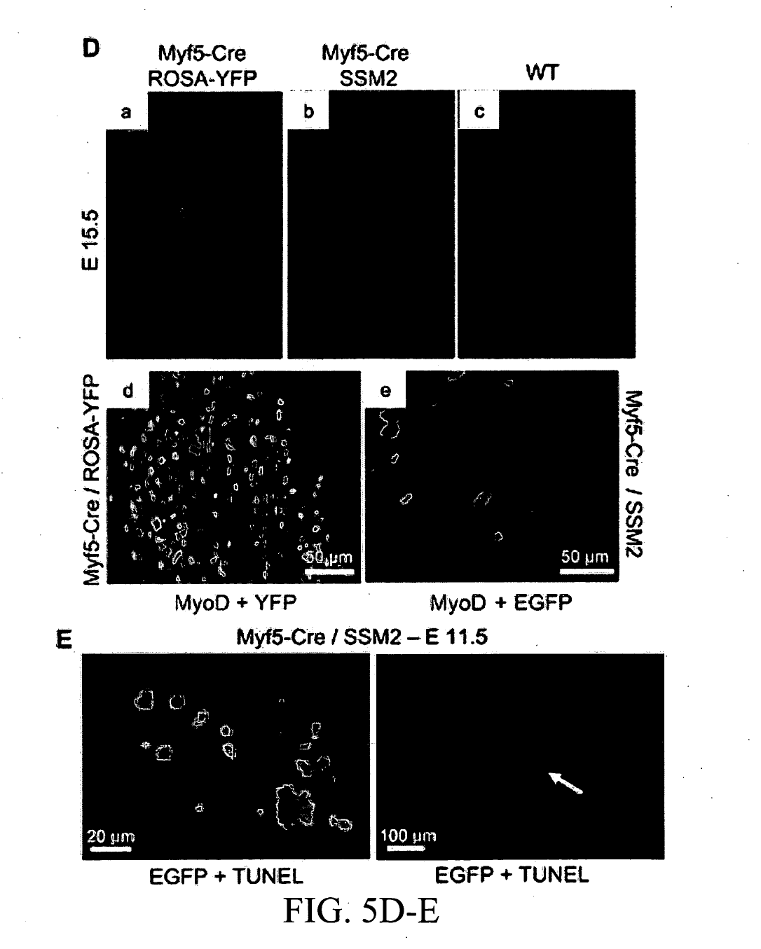

Myf5 Lineage Is a Potential Source of Synovial Sarcoma: In human synovial sarcomas, expression of SYT-SSX takes place from the endogenous SYT promoter, whereas in the disclosed model it takes place from the ROSA promoter within Myf5 lineage. Thus, expression of SYT was evaluated within Myf5 lineage as supporting evidence that Myf5 lineage could be a bona fide source of this tumor. The Myf5-Cre mice were bred to ROSA-YFP reporter mice. The embryos as well as in adult skeletal muscle of Myf5-Cre/ROSA-YFP progenies were evaluated for coexpression of YFP and SYT. SYT was widely expressed within embryos that included almost all YFP-positive Myf5 lineages (FIG. 5A, left panel). Interestingly, SYT expression was also observed in a significant proportion of adult myonuclei (FIG. 5A, right panel). To determine if SYT is expressed in muscle satellite cells, double immunostaining was performed for Pax7 and SYT that did not show any convincing expression of SYT within Pax7-expressing satellite cells.

SYT-SSX2 Expression Restricts Myf5 Lineage Expansion: Synovial-sarcoma-like tumors within Myf5-Cre/SSM2 mice were intensely fluorescent due to expression of EGFP. Surprisingly, however, the surrounding skeletal muscle fibers were mostly GFP negative (FIG. 5B). This observation could have a relatively trivial explanation, such as the IRES driving EGFP expression being inefficient within skeletal muscle as opposed to tumor cells. Alternatively, the explanation could be more biologically profound: the tumors were derived from Myf5-expressing cells, whereas the majority of the skeletal muscle in Myf5-Cre/SSM2 mice, though of normal appearance, were not derived from Myf5-expressing cells.

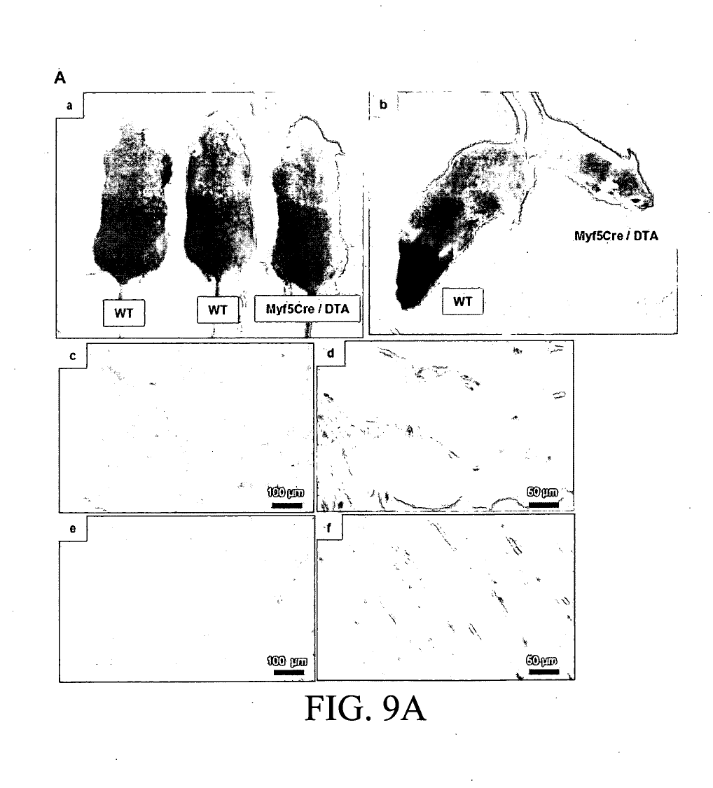

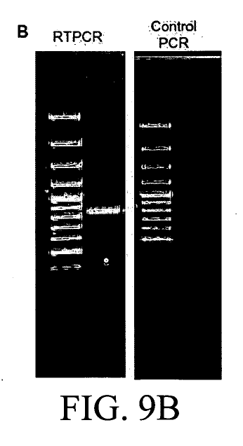

A conditional Hox gene was placed in the ROSA locus followed by the same IRES-EGFP sequence used to construct the SSM2 mice. This mouse was bred to the Myf5-Cre mouse, and the skeletal muscle of the progeny showed readily detectable IRES-driven EGFP expression. Therefore, lack of EGFP expression within skeletal muscle of Myf5-Cre/SSM2 mice was not due to the IRES being inefficient in skeletal muscle, but rather was due to the absence or reduction of the Myf5-derived cell lineage in these mice. This indicates that abrogating or severely restricting the Myf5 lineage is compatible with normal development. A very efficient conditional cell ablation system has been generated in which an attenuated form of the diphtheria toxin gene (DTA-176) is targeted to the ROSA locus and dependent on Cre for its activation (Wu et al., 2006). This mouse was bred to the Myf5-Cre mouse, thereby killing all Myf5-expressing cells in the progeny containing both alleles. Remarkably, these pups were born with apparently normal musculature (FIG. 9A).

Comparative analysis of Myf5-Cre/SSM2 with Myf5-Cre/ROSA-YFP embryos was carried out to analyze Myf5 lineage restriction by SYT-SSX2. This revealed that EGFP-mediated fluorescence within Myf5-Cre/SSM2 embryos at E11.5 (FIG. 5C, middle panel) was comparable to stage-matched Myf5-Cre/ROSA-YFP embryos (FIG. 5C, left panel). However, Myf5 lineage in Myf5-Cre/SSM2 embryos was almost absent by E15.5 (FIG. 5Db) when compared to stage-matched Myf5-Cre/ROSA-YFP embryos (FIG. 5Da). This indicated that SYT-SSX2 expressing Myf5 lineage was not viable. A TUNEL assay was next performed on serial sections of Myf5-Cre/SSM2 embryos at E11.5 (when Myf5 lineage is still detectable), and apoptosis was found within Myf5 lineage (FIG. 5E, left panel). An intriguing observation was that Myf5 lineage near condensing mesenchyme of future rib cartilages did not show apoptosis (FIG. 5E, right panel). This indicates that the microenvironment near cartilages has survival factors preventing apoptosis by SYT-SSX2 that could partly explain the predilection of this tumor to arise near joints containing articular cartilages. Although the Myf5 lineage was restricted by SYT-SSX2 expression, MyoD expression (marker for early skeletal muscle lineage) was normal within Myf5-Cre/SSM2 E15.5 embryos, demonstrating that apoptosis within Myf5 lineage does not compromise skeletal muscle genesis (FIGS. 5Dd and 5De).

Taken together, the above results indicate that SYT-SSX2 induces apoptosis within Myf5 lineage and, only because Myf5-expressing cells are largely dispensable with respect to the formation of skeletal muscle, that Myf5-Cre/SSM mice have the opportunity to develop tumors at later times. Synovial sarcoma induction in the disclosed mouse model appears to be postnatal with a short latency period.

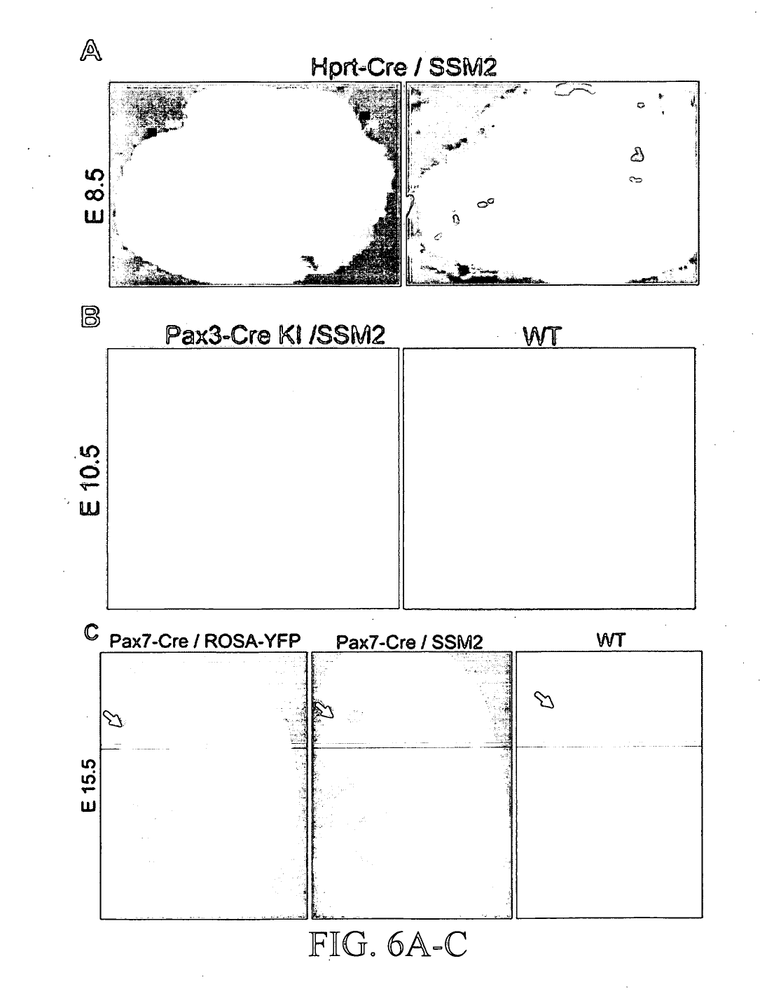

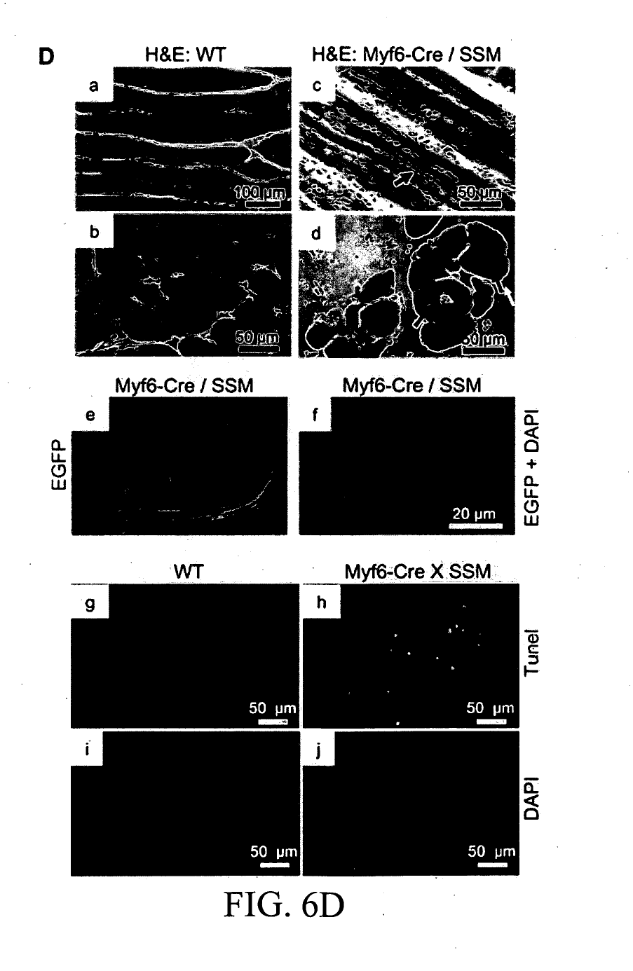

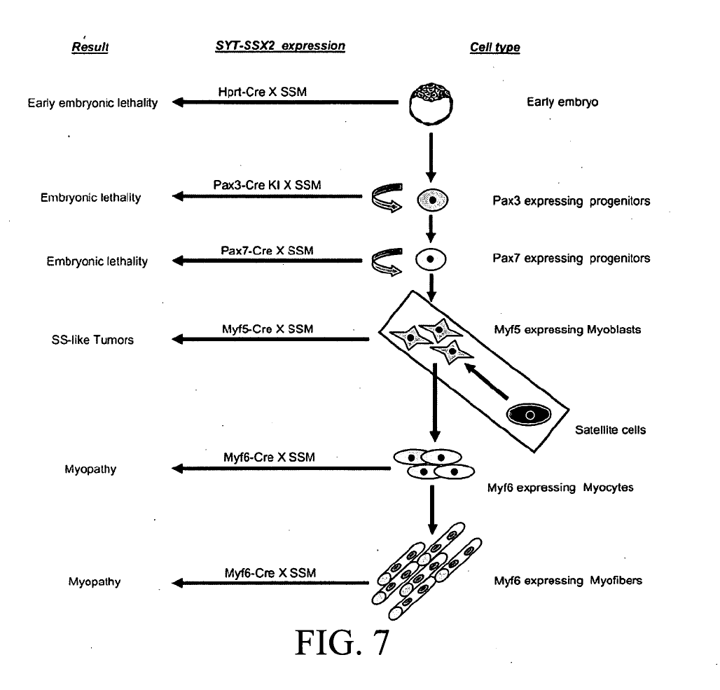

Early Embryonic Expression of SYT-SSX2 Induces Lethality: Next investigated was whether SYT-SSX2 expression within any lineage, if induced early, could also generate synovial sarcoma. To investigate this, conditional SSM mice were bred to Hprt-Cre mice that express Cre in very early-cleavage-stage embryos (Tang et al., 2002). No Hprt-Cre/SSM pups were ever recovered, indicating embryonic lethality. Although attempts to recover Hprt-Cre/SSM embryos failed, highly disorganized EGFP-expressing fetal tissue was detected at E8.5, indicating a dominant disruptive effect of SYT-SSX2 on normal embryonic development (FIG. 6A).

It was next investigated if SYT-SSX2 expression within skeletal muscle progenitors genetically upstream of Myf5, such as those expressing Pax3 and Pax7, leads to synovial sarcoma induction. The conditional SSM mice were bred to Pax3-Cre knockin mice that express Cre recombinase from the endogenous Pax3 locus (Engleka et al., 2005). Pax3 is a transcription factor playing an important role in premigratory neural crest cells, as well as in early precursors of skeletal muscle (Epstein, 2000). No Pax3-Cre/SSM pups were obtained. However, embryos expressing EGFP in Pax3 lineage pattern were recovered at E10.5 (FIG. 6B) but not at E13.5, indicating that embryonic lethality occurred within this developmental time frame.

Next, the SSM mice were bred to mice expressing Cre from Pax7 locus via an IRES placed in the 3′UTR of Pax7 (Keller et al., 2004b). Pax7 is a transcriptional factor that has important functions in skeletal muscle progenitors, particularly in the formation of muscle stem cells (satellite cells) that contribute to postnatal skeletal muscle formation and skeletal muscle regeneration (Jostes et al., 1990; Oustanina et al., 2004). Although no live progenies were obtained, Pax7-Cre/SSM2 embryos were recovered at E15.5, indicating either late-embryonic or perinatal lethality (FIG. 6C, middle panel). Comparison of Pax7-Cre/ROSA-YFP (FIG. 6C, left panel) and Pax7-Cre/SSM2

(FIG. 6C, middle panel) embryos at E15.5 revealed significantly reduced Pax7 lineage within Pax7-Cre/SSM2 based on EGFP fluorescence. However, compared to other regions, a greater proportion of Pax7 lineage was detected in the maxillary and nasal regions of Pax7-Cre/SSM2 embryos (FIG. 6C, arrows). This indicates that SYT-SSX2-expressing Pax7 lineage is mostly not viable, except in proximity to the cartilaginous regions of developing maxilla and nasal turbinates. These observations are in good agreement with those made within Myf5-Cre/SSM2 embryos.

Expression of SYT-SSX2 within Differentiated Cells of Skeletal Muscle Lineage Leads to Myopathy: The effect of inducing SYT-SSX2 expression within more differentiated cells of skeletal muscle lineage were next investigated. Myf6 (Mrf4) is a myogenic regulatory factor expressed within myocytes and myofibers, a population more differentiated than and genetically downstream of Myf5-expressing myoblasts (Chanoine et al., 2004; Pownall et al., 2002). The generation of mice expressing Cre from the Myf6 locus via IRES have been described (Keller et al., 2004a). These Myf6-Cre mice were bred to SSM mice. Although none of the resulting Myf6-Cre/SSM progenies developed tumors, all of them ( 8/8) developed myopathy and eventually died by 6 months of age. No human myopathy has previously been reported to be associated with SYT-SSX.

The myopathy within Myf6-Cre/SSM mice is characterized by abnormal wavy fibers and limited rhabdomyolysis (FIG. 6Dc). Intrafiber vacuolation (FIG. 6Dd, black arrow) and central nuclei (FIG. 6Dd, white arrow) further indicate skeletal muscle damage. Skeletal muscle regeneration was indicated by the presence of myonuclear chains (FIG. 6Dc, arrow). These abnormal skeletal muscle fibers expressed EGFP, indicating expression of SYT-SSX2 (FIGS. 6De and 6Df). Significant apoptosis within these myopathic fibers was detected by TUNEL assay (FIG. 6Dh), indicating that SYT-SSX2 induces apoptosis within differentiated skeletal muscle fibers that leads to myopathy.

### ii. Experimental Procedures

Targeted Mouse Line Production and Genotyping Human SYT-SSX2 cDNA was obtained by RTPCR on total RNA from a synovial sarcoma tumor that was obtained as a de-identified patient sample through an approved University of Utah Institutional Review Board Protocol. This was used to generate targeting vectors.

A clone consisting of an 8.4 kb segment of the Myf5 region including the 3′UTR was isolated from a λ bacteriophage library of mouse strain SvJ-129 (Stratagene). This was used to generate the Myf5-Cre-targeting vector.

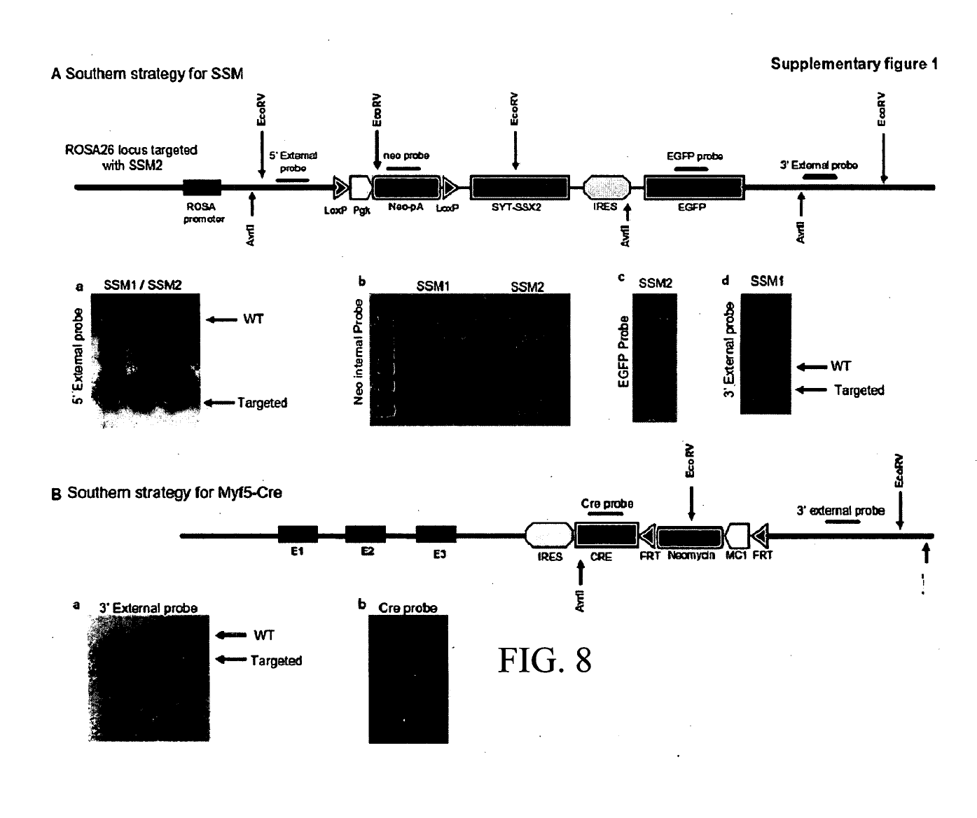

Genotyping was carried out using PCR protocols and Southern blotting outlined in FIG. 8.

Histology and Immunohistochemistry: For histology, specimens were fixed overnight in 4% paraformaldehyde and embedded in paraffin wax following standard procedure. Four to eight micrometer sections were cut and mounted on slides for standard hematoxylin and eosin (H&E) staining, Masson's trichrome staining, or alcian blue staining

Immunohistochemistry on 4 mm sections of paraffin-embedded samples was performed. Counterstaining was done with hematoxylin. For fluorescence-based detection, immunohistochemistry was performed on 8-12 mm frozen sections on samples fixed in 4% paraformaldehyde at 4° C. for 3 hr.

TUNEL Assay: TUNEL assay was performed using a fluorescein In Situ Cell Death Detection Kit (Roche) according to the manufacturer's instructions.

Microarray Analysis: RNA was extracted from tumors of female Myf5-Cre/SSM mice as well as from the skeletal muscle of wild-type age-matched female mice using TRIzol (Invitrogen) and purified using an RNeasy kit (Qiagen). A complete description of microarray analysis is provided in the Supplemental Data. The microarray data have been deposited in NCBI's Gene Expression Omnibus (GEO; www.ncbi.nlm.nih.gov/geo/) and are accessible through GEO Series accession number GSE6461.

Generating Conditional SSM Mice: SYT-SSX2 cDNA was obtained by carrying out RTPCR on RNA from human synovial sarcoma sample using the primers

| | |
|---|---|
| Forward: TGGATGGGCGGCAACATGTCTGTGG | (SEQ ID NO: 17) |
| and | |
| Reverse: GTGAGGGGGGCTTGACCAGGACGCA. | (SEQ ID NO: 18) |

The cDNA was subcloned into NheI-NotI sites of pBIGT plasmid (Srinivas et al., 2001). The PacI-AscI fragement containing Loxp-pgk-Neo-tPA-Loxp-SYT-SSX2-bPA was moved into PacI-AscI cut Prosa26 plasmid to obtain the final SSM1 targeting vector.

To generate the SSM2 targeting vector, the SYT-SSX2 cDNA was moved into SacI-AccI digested pIRES2-EGFP plasmid from BD biosciences. The resulting SYT-SSX2—IRES-EGFP fragment was then moved into NheI-NotI digested pBIGT vector. Subsequently, the Loxp-pgk-Neo-tPA-Loxp-SYT-SSX2—IRES-EGFP-bPA fragment from the pBIGT backbone was removed by digesting with AscI and Pad and placed within AscI-PacI cut Prosa26 vector to obtain the final SSM2 targeting vector.

SSM1 and SSM2 targeting vectors were electroporated into R1 embryonic stem cells and the cells were subjected to positive and negative selection. 76 colonies from each electroporation were analyzed by Southern hybridization using appropriate 5′ external probe, 3′ external probe and two different internal probes (FIG. 8). Seven clones for SSM1 and seven for SSM2 were identified as correctly targeted and cells from one of these were microinjected into C57BL/6 blastocysts to generate chimeric mice. Chimeric mice were mated to C57BL/6 females and their agouti offspring were tested by PCR and Southern hybridization to confirm germ-line transmission of the conditional allele.

Myf5-Cre Mouse Lines: A clone consisting of an 8.4-kb segment of the Myf5 region including the 3′UTR was isolated from a λ bacteriophage library of mouse strain SvJ-129 (Stratagene). An IRES-Cre-FRT-NEO-FRT cassette was then introduced into an engineered AscI site within the 3′UTR of Myf5. A negative selective cassette comprising of thymidine Kinase 1 (TK1) with its own promoter was included in the design (FIG. 1B). The targeting vector was electroporated into R1 embryonic stem cells and the cells were subjected to positive and negative selection followed by southern blot analysis with appropriate 3′ external probe and internal probe (FIG. 8) that identified five out of 152 clones as correctly targeted. One of these was microinjected into C57BL/6 blastocysts to generate chimeric mice. Chimeric mice were mated to C57BL/6 females, and their agouti offspring were tested by PCR and Southern hybridization to confirm germ-line transmission of the conditional allele.

Genotyping: The SSM1 and SSM2 conditional mice were genotyped with the same set of primers:

(SEQ ID NO: 19)

| | |
|---|---|
| Forward-WT: | GTTATCAGTAAGGGAGCTGCAGTGG, |

(SEQ ID NO: 20)

| | |
|---|---|
| Reverse-targeted: | AAGACCGCGAAGAGTTTGTCCTC |
| and | |

(SEQ ID NO: 21)

| | |
|---|---|
| Reverse-WT: | GGCGGATCACAAGCAATAATAACC. |

These primers yielded a 302 bp band for targeted ROSA locus and a 415 bp band for wild type ROSA locus. The PCR conditions were 94° C. for 30 seconds, 59.5° C. for 30 seconds and 72° C. for 30 seconds, for 28-32 cycles.

The various Cre-expressing mice including the Myf5-Cre were genotyped using a pair of Cre specific primers:

| | | |
|---|---|---|
| Forward: | GGATTTCCGTCTCTGGTGTAGC | (SEQ ID NO: 22) |
| and | | |
| Reverse: | ACCATTGCCCCTGTTCACTATC | (SEQ ID NO: 23) |

that yielded a 320 bp band. The PCR conditions were 94° C. for 30 seconds, 60° C. for 30 seconds and 72° C. for 30 seconds, for 28-32 cycles.

A set of Myf5 locus specific primers were also designed to distinguish between heterozygous and homozygous Myf5-Cre that included

| (SEQ ID NO: 24) | |
|---|---|
| Forward-WT: | ACCCTCCAGCTCCAGACTTATC |
| (SEQ ID NO: 25) | |
| Reverse-WT: | CCCTGTAATGGATTCCAAGCTG |
| and | |
| (SEQ ID NO: 26) | |
| Reverse-targeted: | AAAGACCCCTAGGAATGCTC. |

These primers generated a 451 bp wild type product from wild type Myf5 locus and a 594 bp product from the targeted Myf5 locus.

Antibodies: The following primary antibodies were used: Rabbit anti-GFP (Invitrogen—A11122, 1:1000), monoclonal anti-MyoD (BD pharmigen—554130, 1:100), rabbit anti-SYT (Santa Cruz biotech—H-80, 1:100), monoclonal antiskeletal myosin (sigma—M4276, 1:100), monoclonal anti-vimentin (BD Pharmigen—550513, 1:150), monoclonal anti-Bcl-2 (BD Transduction lab—610538, 1:100). Secondary antibodies used were either goat anti-rabbit (alexa546, A11053) or goat anti-mouse (alexa 488, A21121) in various combinations and appropriate conjunction to primary antibodies.

Microarray Hybridization Gene expression was measured using Affymetrix oligonucleotide-based GeneChip® microarray technology (Affymetrix, Santa Clara, Calif.); labeled target RNA was prepared from total tissue RNA using the One-Cycle Target Labeling Kit and Control Reagents (Affymetrix P/N 900493). Total RNA (8 μg) was converted to double-stranded cDNA following priming with an oligo-dT-T7 primer. The resultant cDNA was purified over cDNA Cleanup Spin Columns (Qiagen, Valencia, Calif.). The purified cDNA was subjected to in vitro transcription using T7 RNA polymerase in the presence of biotinylated UTP. The resultant cRNA was purified with an RNeasy column (Qiagen, Valencia, Calif.), eluted in H2O, and quantified by UV spectrophotometry. cRNA (15 μg) was fragmented following the Affymetrix protocol, added to 270 μl of hybridization buffer, and hybridized to the Affymetrix GeneChip® Mouse Genome 430 2.0 arrays (P/N 900495). After 20 hr of hybridization at 45° C., the GeneChips® were washed, stained, and scanned according to the standard Affymetrix protocol. The arrays were scanned using an Affymetrix GeneChip Scanner 3000 enabled for High-Resolution Scanning, and the raw images were converted to CEL files using Affymetrix GCOS software.

Preprocessing and Rescaling: The raw expression data as obtained from Affymetrix's GeneChip were normalized and re-scaled to account for different chip intensities using GenePattern 2.0's Expression File Creator module. The MASS algorithm was used to accomplish the mean scaling normalization and scaling to 500. Following scaling, the data sets were converted into the .gct file format for further use in the GenePattern analysis programs. The complete data sets are available online at www.ncbi.nlm.nih.gov/geo/query/acc.cgi?acc=GSE6461.

Hierarchical Clustering Analysis was performed using the Hierarchical Clustering module available in GenePattern 2.0. The following parameters were used to derive the dendrogram. The data were row normalized, and then Euclidean distance via pairwise average-linkage was measured. The dedrogram was generated as described (M. B. Eisen, et al, PNAS 1998)

Human Sarcoma Data Set: This publicly available data set was generated from microarray analysis of 181 human sarcoma patient samples at the National Human Genome Research Institute (NHGRI). The data set includes 16 human sarcoma tumor types: 1 alveolar soft part sarcoma, 1 chondrosarcoma, 1 clear cell sarcoma, 5 dermatofibrosarcomas, 20 Ewing's sarcomas, 7 fibrosarcomas, 5 gastrointestinal stromal tumors, 6 leiomyosarcomas, 33 liposarcomas, 38 malignant fibrous histiocytomas, 6 malignant hemangiopericytomas, 6 malignant peripheral nerve sheath tumors, 2 mixed Mullerian tumors, 6 osteosarcomas, 6 rhabdomyosarcomas, 10 sarcomas (NOS), 3 benign schwannomas, and 18 synovial cell sarcomas. The microarray platform used was a cDNA array containing 12601 cDNA clones annotated with Integrated Molecular Analysis of Genomes and their Expression (IMAGE) CloneIDs. The complete data set was downloaded from www.ncbi.nlm.nih.gov/geo/gds/gds_browse.cgi?gds=1268. Baird et al. reported specific upregulated “gene sets” for several tumor types. These gene sets were used for analyses. These data were downloaded from watson.nhgri.nih.gov/sarcoma/Table %20A_AllTumor_GeneList.xls

Human Sarcoma Data Set: This publicly available data set was generated from microarray analysis of 39 human tumor samples. The data set includes 7 human sarcoma tumor types and 15 comparator normal human tissues: brain, stomach, colon, pancreas, prostate, skin, small intestine, adrenal, connective tissue, heart, kidney, liver, lung, skeletal muscle, spleen, 7 fibrosarcomas, 2 Gastrointestinal Stromal Tumors, 6 Leiomyosarcomas, 4 Liposarcoma dediffs, 3 Liposarcoma pleomorphics, 9 Malignant Fibrous Histiosarcomas, 4 Round cell sarcomas, 4 Synovial sarcomas. The profiling experiments were performed using Affymetrix HG U133A (human) oligonucleotide arrays. The complete data set was downloaded from www.ncbi.nlm.nih.gov/geo/query/acc.cgi?acc=GSE2719

Human Mesenchymal Tumor Data Set: This publicly available data set was generated from microarray analysis of 96 mesenchymal tumors, representing 19 different subtypes from specimens resected at the London Bone and Soft Tissue Tumour Service (Royal National Orthopaedic Hospital, Stanmore and University College London Hospitals, London), Great Ormond Street Hospital, London, or the Nuffield Orthopaedic Center, Headington, Oxford, in the UK. The data set includes 4 alveolar rhabdomyosarcomas (3 PAX3-FKHR, 1 NA), 4 chondroblastomas, 4 chondromyxoid fibromas, 7 chondrosarcomas, 4 chordomas, 3 dedifferentiated chondrosarcomas, 3 embryonal rhabdomyosarcomas, 5 Ewing's Sarcomas (all EWS-FLI), 5 fibromatoses, 8 leiomyosarcomas, 3 lipomas, 4 malignant peripheral nerve sheath tumors, 10 monophasic synovial sarcomas (1S)-T-SSX NOS, 1 SYT-SSX2, 2 SYT-SSX1, 6 NA), 7 myxoid liposarcomas (4 CHOP/FUS, 3 NA), 4 neurofibromas, 11 osteosarcomas, 3 undifferentiated sarcomas, 4 schwannomas, and 3 welldifferentiated liposarcomas. The profiling experiments were performed on Affymetrix HG-U133A Human GeneChips. The RMA algorithm was used for pre-processing, normalizing and calculation of expression values. The complete data set was downloaded from www.ebi.ac.uk/aerep/dataselection?expid=484703006.

Human Sarcoma Data Set: This publicly available data set was generated from microarray analysis of 41 tissue tumours from soft-tissue tumour specimens resected at the Vancouver Hospital and Health Sciences Center, the Stanford University Medical Center, and the Hospital of the University of Pennsylvania between 1993 and 2000. The data set includes eight gastrointestinal stromal tumours; eight monophasic synovial sarcomas; four liposarcomas (one dedifferentiated, one myxoid, two pleomorphic); 11 leiomyosarcomas (including one primary and metastatic pair); eight malignant fibrous histiocytomas; and two benign peripheral nerve-sheath tumours (schwannomas). Singular value decomposition and ANOVA were used to identify and correct for bias introduced by different array types. The profiling experiments were performed using NHGRI human 22 000 (22K) spotted cDNA microarrays. Five specimens for which adequate amounts of mRNA were available were analysed on both 22K and 42K gene arrays. The complete data set was downloaded from www.ebi.ac.uk/aerep/dataselection?expid=826112952 and genome-www.stanford.edu/sarcoma/.

Probe Matching across Data Sets: Because the data sets used in the disclosed analyses were generated using different microarray platforms, the annotation was converted to UniGene identifiers to facilitate direct comparison. For comparing between human and mouse data sets, human UniGene IDs were used as the common identifier. For mouse-to-mouse comparisons, mouse UniGene IDs were used.

Conversion of Affymetrix Accession Number to UniGenelD: The disclosed microarray data, as well as the Detwiller et al data sets were annotated by Affymetrix accession numbers (Detwiller et al., 2005). The Affymetrix accession numbers were matched to their corresponding human UniGeneIDs via the GeneCruiser ver.4 module available in GenePattern.

Conversion of Human IMAGE ID to UniGenelD: The human sarcoma and SRBCT data sets were annotated by IMAGE IDs. These IMAGE IDs were matched to their corresponding UniGeneIDs via the relations established by the NCBI in the UniGene repository data set (ftp.ncbi.nih.gov/repository/UniGene/Homo_{—}sapiens/Hs.data.gz, build date Jul. 18, 2006).

Annotation of Mouse Microarray Data with Human UniGeneIDs: To convert the mouse annotation of gene sets derived from our SAM data to human UniGeneIDs both mouse and human UniGene databases were used. The mouse database (ftp.ncbi.nih.gov/repository/UniGene/Mus_{—}musculus/Mm.data.gz, build date Jul. 18, 2006) was first used to match the NCBI accession numbers from the disclosed gene sets to their mouse UniGenelD and corresponding most homologous human ProteinID Conveniently, the mouse UniGene database contains a “best match” homologous human ProteinID for each mouse UniGene entry. This homologous human ProteinID was used to then find the appropriate human UniGenelD from the human UniGene database.

Supervised Analysis: As a preliminary analysis genes were rank-ordered using a supervised methodology to identify those genes which were differentially expressed between the disclosed MYF-5 CRE/SSM2 tumors and WT mouse skeletal muscle, the Detwiller et al data set (human synovial sarcoma tumor samples and “normal” human tissue (connective tissue, skeletal muscle and cardiac muscle)), and the Baird et al., Henderson et al., and Nielsen et al. data sets (human synovial sarcoma tumor samples versus other human tumor samples). The complete data sets were first filtered (after scaling, and applying threshold and ceiling values of 10 and 16000 respectively). Probe sets that had at least a 3-fold change across at least two samples were retained. This is similar to the filtering values that have previously used (Lessnick et al., 2002) and usually provides an appropriate balance between minimizing false-positive and false-negative expression changes when compared to other methods of gene expression detection. For each data set a reference pattern was chosen based on the presence of either the phenotype of interest (SYT/SSX model tumors or synovial cell sarcoma), or control (normal tissue or other sarcomas). A two class distinction was thus generated. The “synovial signature” class consists of genes whose expression levels are increased in the either the SYT/SSX model tumors or synovial cell sarcoma samples, and are thus presumptively “upregulated” by SYT/SSX. The reverse class consists of genes whose expression levels are decreased in the presence tumor samples, and are thus presumed to be “downregulated” by SYT/SSX.

The reference pattern utilizes all experimental runs. Thus, the different experimental samples and replicates are neither blended nor averaged, but rather treated separately. Genes correlated with the particular class distinctions (e.g. “synovial signature”) were identified by sorting all of the genes on the array according the signal-to-noise statistic (μclass 0-μclass 1)/(σ class 0+σclass1) where μ and σ represent the mean and standard deviation of expression, respectively, for each class (Golub et al., 1999). 1000 random permutations of the column (sample) labels were performed to compare these correlations to what would be expected by chance. Permutation testing allowed for selection of significantly correlated genes at a p<0.01. All analyses were performed using the Class Neighbors module in GenePattern 2.0.

Spearman Correlation of Rank-Ordered Lists: One difficulty in the comparison of microarray data generated in different experiments, or on different microarray platforms, is that the raw data may not be directly comparable. To overcome this difficulty in comparing the rankordered gene lists derived from the supervised analysis described above, Spearman's rank correlation coefficient testing was used. Ordered lists of genes significantly correlated (p<0.01) with the “synovial signature” were converted to ranks, and the differences D between the rank of each gene in the list were calculated as follows:

$$ρ = 1 - \frac{6 \sum {D}^{2}}{N ( {N}^{2} - 1 )}$$

where:

D=the difference between the ranks of corresponding values of X and Y, and

N=the number of pairs of values.

Permutation testing was accomplished as the lists were then randomly reordered 10,000 times, with resulting scores being compared to the experimental parameters ultimately yielding a p value. These permutations were performed to determine if the determined correlation coefficient was greater than would be expected by chance alone. The proportion of times that a correlation coefficient for the permuted gene list was equal to, or greater than, the correlation coefficient for the experimental data sets was reported as the p value. Table 4 also reports the number of genes that were used in the equation.

Significance Analysis of Microarray: SAM (Tusher et al., 2001) was used as instituted in the TM4 MeV software (Saeed et al., 2003) to identify the probes differentially regulated between MYF-5 CRE/SSM2 tumors and normal skeletal muscle on the microarray. The data was first limited to those genes showing a fold change of at least 3. Delta (tuning) values were then chosen for each analysis to identify clones at a false discovery rate (FDR) of <0.05 against 1000 random permutations. The genes identified as being upregulated between MYF-5 CRE/SSM2 tumors and normal skeletal muscle were designated as the “SYT/SSX model gene set.”

Extraction of Human Synovial Cell Sarcoma Gene Set: For the Baird et al data sets, a representative “synovial cell sarcoma up-regulated” gene lists has been published. However, this list was not published specifically for synovial cell sarcoma in the Detwiller et al publication. In order to test the hypothesis that a SAM derived synovial signature would be enriched across experiments when performing GSEA, similar parameters were used to extract a synovial cell sarcoma upregulated gene set from the Detwiller et al data set. As in the previously outlined analysis of MYF-5 CRE/SSM2 tumors, SAM was used with delta values for each analysis to select upregulated genelists with a FDR of <0.01 and fold change >2.

Gene Set Enrichment Analysis: GSEA is a statistical approach that analyzes the distribution of a defined set of genes (the “gene set”) against a separate rank-ordered gene list (Subramanian et al., 2005). The null hypothesis is that the genes in the gene set are randomly distributed through the rank-ordered list. Rejection of the null hypothesis indicates that the gene set is preferentially enriched near the top (or the bottom) of the rank-ordered list, indicating significant similarity (or dissimilarity) between the gene set and the ordering of the rank-ordered list (Subramanian et al., 2005).

Comparison of SYT/SSX Model Gene Set to Human Tumor Rank-Ordered List: GSEA was first used to test for enrichment of our SYT/SSX model signature in human synovial sarcoma. A signal-to-noise (SNR) analysis was used with 1000 random permutations of a human data set (Detwiller human sarcoma) as instituted in javaGSEA for the rank-ordered list. The rank-list analysis was classed to compare synovial sarcoma samples versus all others. The previously described SAM derived set of differentially upregulated genes from the Myf5-CRE/SSM2 tumor expression data (“SYT/SSX model gene set”) was used as the comparator gene set in the enrichment analyses. The GSEA showed enrichment of a number of the SYT/SSX model genes in human synovial cell sarcoma, but overall the gene set enrichment was not statistically significant.

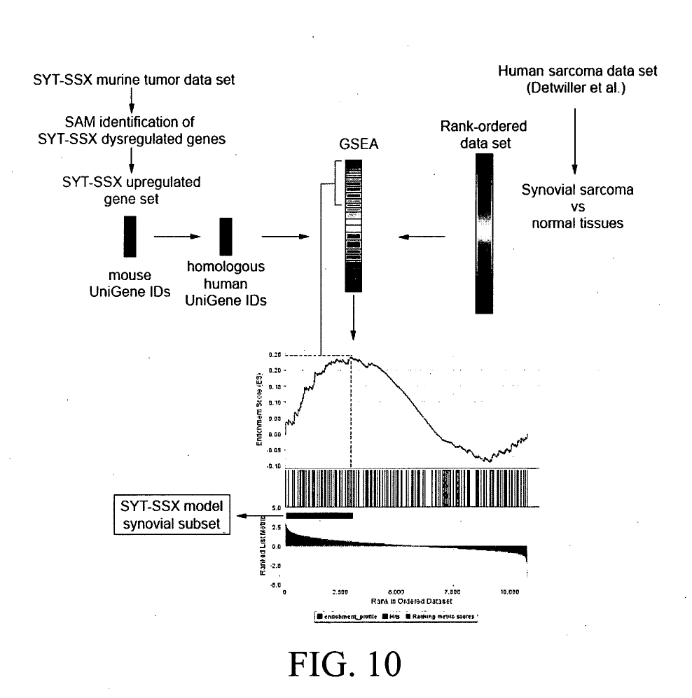

Extraction of the “SYT/SSX Model Synovial Subset”: Because the GSEA described above identified a subset of genes that were enriched in the “test” synovial sarcoma data set (Detwiller et al) but which was not statistically significant, these enriched genes can constitute a smaller human synovial cell tumor specific signature within the larger murine tumor signature. To test this, genes were first extracted from the murine tumor signature which showed enrichment in the Detwiller et al data set as identified by GSEA (FIG. 10). This smaller gene list was designated as the “SYT-SSX model synovial subset” and this set was used in along with the full gene set in subsequent analyses.

Comparison of SYT/SSX Model Gene Set, SYT/SSX Model Synovial Subset and Human Synovial Signature Gene Set to Human Tumor Rank-Ordered List: A signal-to-noise (SNR) analysis was again used with 1000 random permutations of the Baird et al. human sarcoma data set, Henderson et al. mesenchymal tumor data set, and Nielsen et al. human sarcoma data sets respectively as instituted in javaGSEA for the rank-ordered lists in this GSEA. Each rank-list analysis was classed individually according to tumor type (e.g. synovial sarcoma versus others). The previously described SAM derived set of SYT/SSX model gene set, the newly extracted SYT/SSX model synovial subset and the Detwiller data set were used as the comparator genes sets in the enrichment analyses. The enrichment scores (ES), normalized enrichment scores (NES—which corrects for multiple testing and the bias introduced by difference in gene set size) and family-wise error rate (p value) were derived as described (Subramanian et al., 2005).

TABLE 2
GSEA of Gene Sets Upregulated in SYT-SSX and Synovial Cell Sarcoma Tumors Compared to Human Sarcoma Data Sets

| | | | | | | | Human Tumor | | |
|---|---|---|---|---|---|---|---|---|---|
| | SYT-SSX Model | | | SYT-SSX Model | | | Synovial Cell | | |
| | Gene Set | | | Synovial Subset | | | Sarcoma Gene Set | | |
| | | | FWER | | | FWER | | | FWER |
| Human Cancer Phenotype Data Set | ES | NES | p Value | ES | NES | p Value | ES | NES | p Value |

Baird et al.

| | | | | | | | | | |
|---|---|---|---|---|---|---|---|---|---|
| Synovial cell sarcoma | 0.167 | 0.780 | 0.791 | 0.582* | 1.945* | 0.001* | 0.539* | 2.030* | 0.001* |
| Fibrosarcoma | −0.230 | −1.084 | 0.379 | 0.352 | 1.184 | 0.364 | −0.206 | −0.749 | 0.713 |
| Ewing's sarcoma | −0.160 | −0.731 | 0.739 | 0.213 | 0.706 | 0.790 | 0.314 | 1.151 | 0.368 |
| Osteosarcoma | −0.235 | −1.102 | 0.345 | −0.235 | −0.782 | 0.708 | −0.187 | −0.672 | 0.765 |
| Rhabdomyosarcoma | 0.182 | 0.854 | 0.739 | −0.254 | −0.848 | 0.627 | 0.209 | 0.756 | 0.799 |
| Leiomyosarcoma | 0.241 | 1.123 | 0.415 | −0.269 | −0.911 | 0.587 | −0.152 | −0.558 | 0.799 |
| Liposarcoma | 0.201 | 0.932 | 0.609 | −0.405 | −1.365 | 0.155 | −0.346 | −1.285 | 0.224 |
| Malignant fibrous histiosarcoma | −0.178 | −0.831 | 0.668 | −0.456 | −1.572 | 0.048 | −0.385 | −1.424 | 0.116 |

Henderson et al.

| | | | | | | | | | |
|---|---|---|---|---|---|---|---|---|---|
| Monophasic synovial cell sarcoma | −0.293 | −1.208 | 0.216 | 0.626* | 1.565* | 0.022* | 0.712* | 1.783* | <0.001* |
| Alveolar rhabdomyosarcoma | 0.272 | 1.106 | 0.372 | 0.493 | 1.236 | 0.224 | 0.526 | 1.301 | 0.168 |
| Ewing's sarcoma | 0.276 | 1.127 | 0.342 | 0.476 | 1.185 | 0.266 | 0.327 | 0.818 | 0.702 |
| Myxoid liposarcoma | −0.243 | −0.989 | 0.470 | 0.418 | 1.060 | 0.439 | −0.280 | −0.699 | 0.764 |
| Dedifferentiated chondrosarcoma | 0.369 | 1.497 | 0.039 | 0.378 | 0.955 | 0.571 | 0.331 | 0.817 | 0.736 |
| Embryonal rhabdomyosarcoma | 0.236 | 0.974 | 0.524 | 0.358 | 0.927 | 0.584 | 0.603 | 1.501 | 0.042 |
| Chondroblastoma | 0.259 | 1.057 | 0.433 | 0.307 | 0.766 | 0.740 | −0.393 | −0.968 | 0.513 |
| Fibromatosis | −0.253 | −1.037 | 0.421 | −0.303 | −0.764 | 0.743 | −0.354 | −0.882 | 0.609 |
| Well-differentiated liposarcoma | −0.242 | −0.982 | 0.525 | −0.327 | −0.812 | 0.701 | −0.535 | −1.321 | 0.121 |
| Lipoma | 0.226 | 0.925 | 0.598 | −0.330 | −0.843 | 0.680 | −0.572 | −1.375 | 0.102 |
| Osteosarcoma | 0.325 | 1.316 | 0.142 | −0.364 | −0.903 | 0.623 | −0.353 | −0.874 | 0.651 |
| Neurofibroma | −0.206 | −0.837 | 0.697 | −0.444 | −1.113 | 0.349 | −0.495 | −1.226 | 0.230 |
| Leiomyosarcoma | 0.277 | 1.142 | 0.318 | −0.504 | −1.265 | 0.163 | −0.379 | −0.976 | 0.492 |
| Chondrosarcoma | −0.237 | −0.966 | 0.529 | −0.511 | −1.300 | 0.189 | −0.442 | −1.094 | 0.380 |
| Chondromyxoid fibroma | −0.270 | −1.093 | 0.379 | −0.543 | −1.392 | 0.098 | −0.448 | −1.104 | 0.360 |
| Chordoma | −0.330 | −1.344 | 0.132 | −0.566 | −1.372 | 0.107 | −0.551 | −1.369 | 0.111 |
| Schwannoma | 0.281 | 1.159 | 0.296 | −0.598 | −1.495 | 0.048 | −0.555 | −1.358 | 0.121 |

Nielsen et al.

| | | | | | | | | | |
|---|---|---|---|---|---|---|---|---|---|
| Synovial cell sarcoma | −0.293 | −0.942 | 0.566 | 0.828* | 1.568* | 0.044* | 0.679* | 1.842* | 0.001* |
| Malignant fibrous histiosarcoma | 0.400 | 1.267 | 0.295 | 0.462 | 0.868 | 0.669 | −0.511 | −1.423 | 0.125 |
| Gastrointestinal stromal tumor | −0.292 | −0.962 | 0.517 | 0.444 | 0.817 | 0.695 | −0.357 | −0.985 | 0.498 |
| Schwannoma | 0.435 | 1.307 | 0.215 | −0.437 | −0.816 | 0.635 | −0.394 | −1.026 | 0.454 |
| Leiomyosarcoma | 0.244 | 0.793 | 0.648 | −0.634 | −1.152 | 0.406 | −0.275 | −0.753 | 0.785 |
| Liposarcoma | −0.283 | −0.916 | 0.600 | −0.745 | −1.376 | 0.166 | −0.173 | −0.480 | 0.807 |

Enrichment of genes upregulated in the mouse model and in human synovial sarcoma was analyzed by GSEA in human sarcoma data sets.
The mouse model “synovial subset” comprises genes from the model set that show enrichment in an independent human sarcoma data set.
Positive ES scores indicate enrichment in the cancer phenotype; negative ES scores indicate enrichment in the comparator class (“antienrichment”).
Asterisks indicate statistical significance.
As expected, the human synovial sarcoma gene set showed enrichment in human synovial sarcoma (positive ES score, significant FWER p value). Likewise, the mouse model “synovial subset” showed significant enrichment in human synovial sarcoma, while the full mouse model gene set showed nonsignificant enrichment. Neither the full mouse model gene set nor the synovial subset were significantly enriched in other human cancers.
ES, enrichment score;
NES, normalized enrichment score;
FWER, family-wise error rate.

TABLE 3
Frequency of tumors at different anatomical locations.

| Anatomical site | Frequency | Size | Notes |
|---|---|---|---|
| Intercostal region | ~100% | 0.1 to 0.5 cms. | Arise from intercostals |
| | | | musculature and not the |
| | | | ribs. |
| Limbs | ~95% | 0.5 to 1.0 cms | Always close to joints. |
| | | | Does not arise from |
| | | | the joint. |
| Other Locations | ~5% | 0.5 to 1.0 cms | Always arise from |
| Esophagus | | | musculature. |
| Back Musculature | | | |
| Neck Musculature | | | |

Tumor detection at necropsy was based on gross detection and EGFP based fluorescence detection. Tumors had chalky white appearance distinct from surrounding tissue making gross detection easy while the detection of smaller tumors were facilitated by EGFP mediated fluorescence. Multiple tumors were detected in all animals analyzed and the number of tumors detected was a function of time such older mice had more tumor load. Tumors arising from the intercostals musculature were small and more localized compared to tumors within limb musculature that were much larger with areas of hemorrhage and necrosis.

TABLE 4
Spearman's rank correlation of SYT-SSX model gene set to
human synovial cell sarcoma

| Human Cancer Phenotype data sets | n | overlap | ρ (rho)* | p |
|---|---|---|---|---|
| Nielsen et al Synovial sarcoma vs | 1554 | 33 | 0.278 | <0.0001 |
| soft tissue tumors | | | | |
| Baird et al Synovial sarcoma vs | 1832 | 201 | 0.164 | <0.0001 |
| other sarcomas | | | | |
| Detwiller et al Synovial sarcoma vs | 3874 | 475 | 0.056 | <0.0001 |
| normal tissues | | | | |
| Henderson et al Monophasic Synovial | 5851 | 140 | −0.018 | <0.0001 |
| sarcoma vs mesenchymal tumors | | | | |

All genes significantly correlated with the SYT-SSX murine tumor phenotype (n = 3572 p < 0.01) in our expression data were rank ordered as described in the text and compared to similarly rank ordered data from human tumor profiles (Baird et al., 2005, Detwiller et al., 2005, Henderson et al., 2005, Nielsen et al., 2002). The Spearman correlation coefficient (ρ) is shown. The p value was an empirically determined value based on 10,000 randomly generated rank orders of the same data. The number of common genes indicates how many genes were present in each pairwise comparison.

### 2. Example 2

As disclosed herein, sporadic random expression of SYT-SSX2 within multiple tissue types leads exclusively to generation of synovial sarcomas with evidence of non-myogenic origin in some tumors indicating multiple cell of origin. This new mouse model recapitulates the spectrum of human synovial sarcomas. Ubiquitous expression of SYT-SSX2 in most tissue proved lethal indicating requirement of more discreet expression than achieved with traditional Cre-expressing mice. The disclosed strategy of random expression circumvents this problem and can be adapted for rapidly generating other translocation-associated sarcomas in mice.

Disclosed herein is the generation of a mouse line (SSM2 mouse line) expressing human synovial sarcoma-associated SYT-SSX2 fusion protein conditionally from the ROSA locus. Within the skeletal muscle lineage, the biological effects of SYT-SSX2 expression were found to be dependent upon the position of the cell in the genetic hierarchy of muscle lineage. While its expression in immature but committed myoblasts generated synovial sarcomas, expression in genetically upstream progenitor cell populations led to embryonic lethality and expression in genetically downstream differentiated skeletal muscle fibers led to myopathy. Although this demonstrated that myoblasts could give rise to synovial sarcomas, it did not rule out other potential sources. If there are multiple sources, the cell of origin can influence tumor phenotype to various degrees; 1) the origin can influence the gene expression profile of histologically similar tumors and influence the clinical course/prognosis or 2) a more dramatic effect where SYT-SSX2 may give rise to histologically distinct tumors depending on the origin.

i. Results

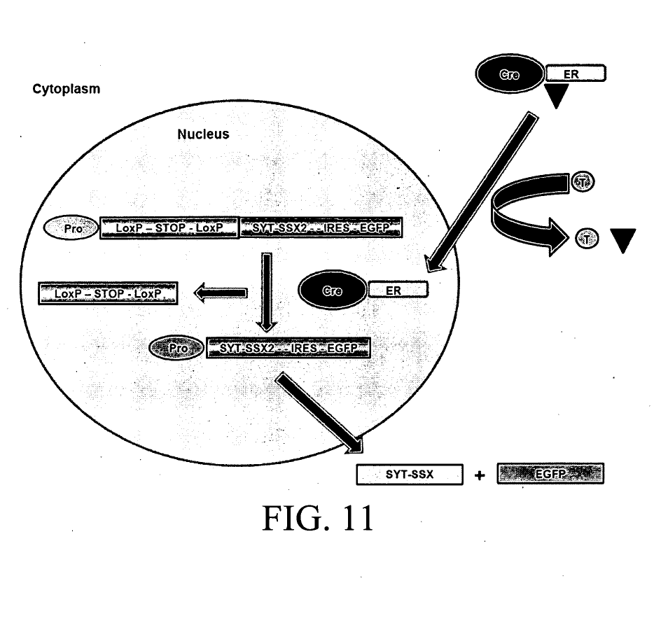

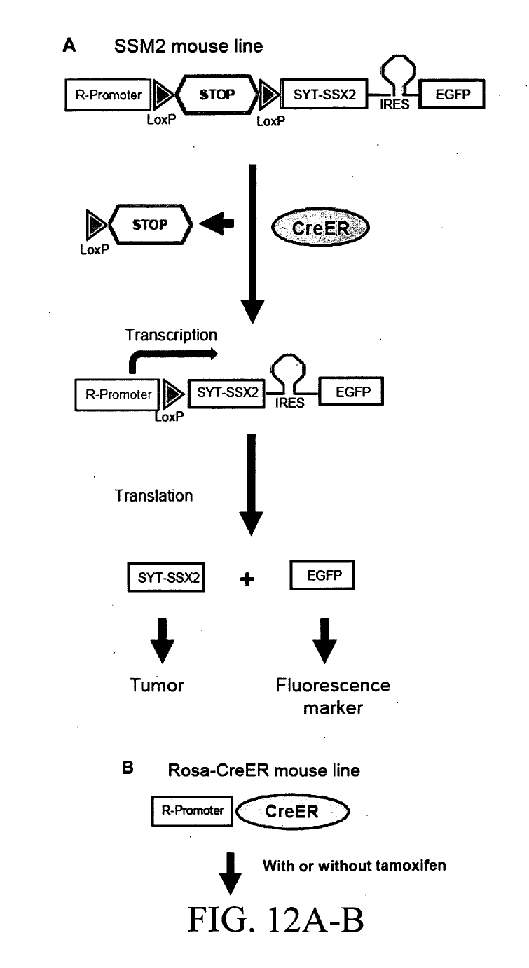

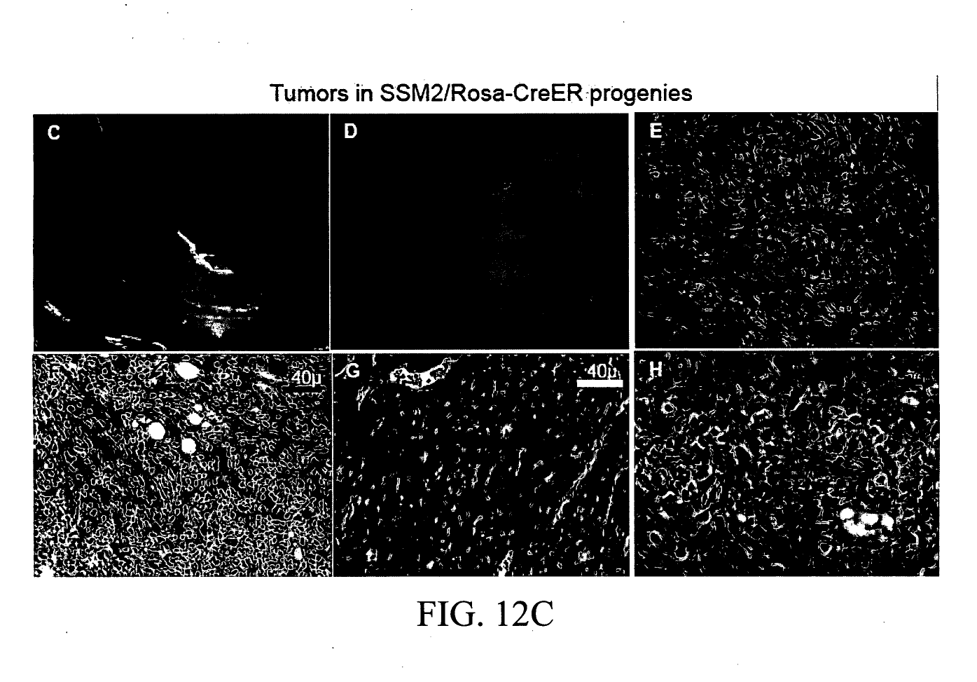

Early ubiquitous expression of SYT-SSX2 is not compatible with embryogenesis. A dominant pro-apoptotic effect of SYT-SSX2 expression leads to an early lethality in most breeding experiments with “straight Cre drivers” indicating a requirement of more discreet expression within specific tissue to uncover all potential origins of this tumor. SYT-SSX2 expression can be confined to a subset of Cre expressing cells by using the tamoxifen-inducible CreER system (FIG. 11). Expressing CreER fusion protein, instead of straight Cre, in specific tissues provides for an additional temporal control over Cre activity along with tissue specificity by allowing control over nuclear entry of Cre recombinase via exogenous application of tamoxifen. A ROSA-CreER mouse line that expresses CreER ubiquitously was obtained and bred to the conditional SSM2 mouse line (FIGS. 12A and 12B). The number of cells where Cre is translocated to the nucleus (and hence expressing SYT-SSX2) within the ROSA-CreER/SSM2 progenies can be controlled by the amount and frequency of exogenous application of tamoxifen. The ROSA-CreER/SSM2 progenies were divided into two groups; those exposed to a single injection of 150 μl of 20 mg/gm tamoxifen those not exposed to tamoxifen. While both tamoxifen exposed and unexposed ROSA-CreER/SSM2 mice harbored multiple tumors at various time after the age of 8 months, none of the control mice (ROSA-CreER, SSM2, and WT) showed the presence of any tumors or other phenotype. The ROSA-CreER mouse is known to be ‘leaky’, where some background level of nuclear translocation of Cre is observed even in the absence of any tamoxifen. This explains the development of tumors even in the absence of tamoxifen exposure.

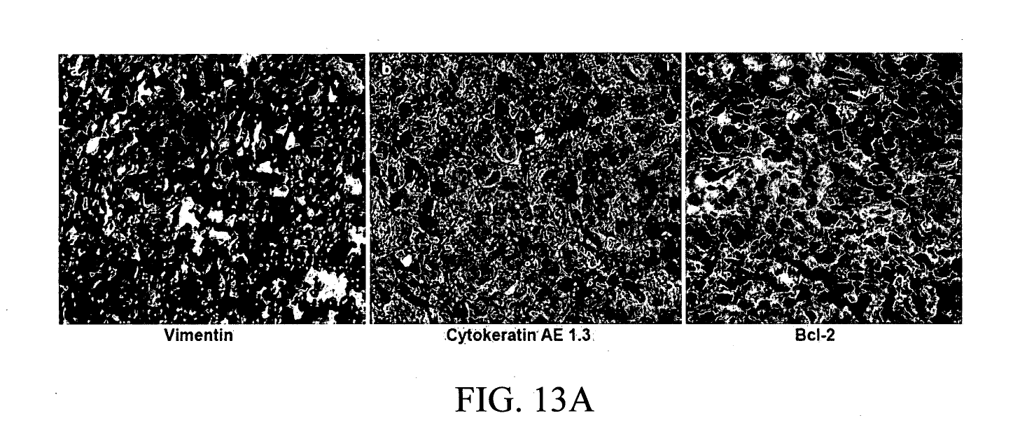

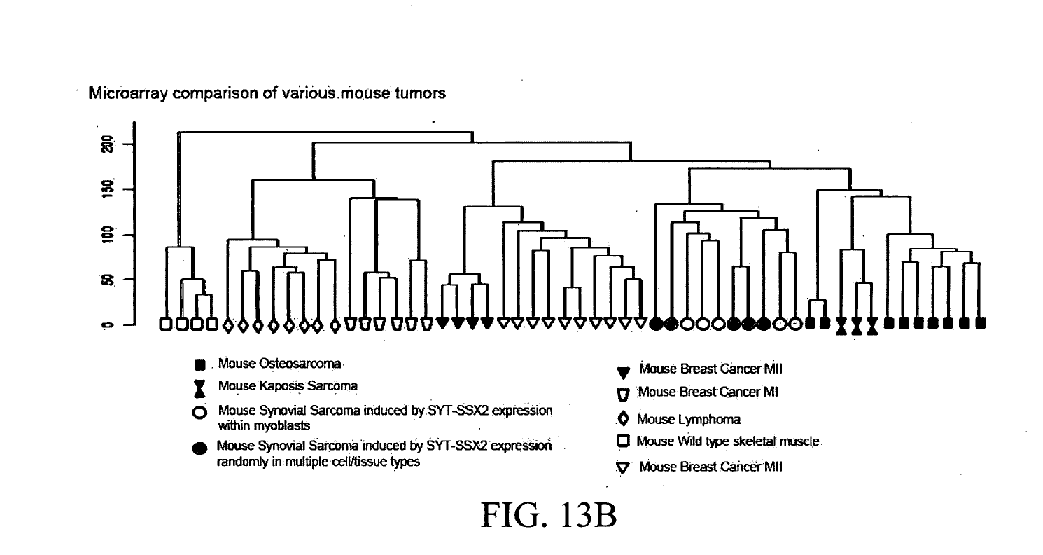

Tumors in ROSA-CreER/SSM2 mice were detected within different anatomic locations such as face, tail, limbs, subcutaneous, etc that suggest multiple cell of origin in this model in contrast to the herein disclosed model of synovial sarcomas induced only within skeletal musculature of mice. The tumors were intensely fluorescent demonstrating expression of the SYT-SSX2 fusion protein (FIGS. 12C and 12D). EGFP expression (and hence SYT-SSX2 expression) is further demonstrated at the cellular level in a micrograph (FIG. 12E). The tumor histology strikingly mimics the histology of human synovial sarcomas (FIG. 12F to 12H). Immunohistochemical analysis also demonstrated expression of antigens that are usually over-expressed in human synovial sarcomas (FIG. 13Aa to 13Ac) further highlighting the similarity of the mouse tumors to human synovial sarcomas.

To investigate more into the similarity of the newly generated mouse synovial sarcoma-like tumors with human synovial sarcomas in terms of transcriptional profile, total RNA was extracted from the newly generated tumors and microarray hybridization carried out using the Affymetrix mouse whole genome 430 2.0 gene chips. Published and freely available microarray data were downloaded using the same gene chip on various mouse tumors from the NCBI Gene Expression Omnibus (GEO) website and an expression based clustering carried out using the Genesifter Website (FIG. 13B). This revealed that the newly generated mouse sarcomas cluster tightly with the previously generated mouse synovial sarcomas and not with other mouse tumors such as osteosarcomas, breast cancer, multiple myeloma etc (FIG. 13B) thereby demonstrating that the newly generate mouse tumors are indeed synovial sarcomas.

In summary, disclosed herein is the successful generation of mouse synovial sarcomas using a strategy of random sporadic conditional induction of translocation-derived chimeric oncogene. Such a model has the great advantage of recapitulating multiple cell of origin within a single model system making it more robust for pre-clinical applications than the traditional single-origin conditional models. Furthermore, this simple strategy can be easily applied to model other translocation-associated sarcomas without a prior knowledge of the cell of origin.

### F. REFERENCES

Baird, K., Davis, S., Antonescu, C. R., Harper, U. L., Walker, R. L., Chen, Y., Glatfelter, A. A., Duray, P. H., and Meltzer, P. S. (2005). Gene expression profiling of human sarcomas: Insights into sarcoma biology. Cancer Res. 65, 9226-9235.
Chanoine, C., Della Gaspera, B., and Charbonnier, F. (2004). Myogenic regulatory factors: Redundant or specific functions? Lessons from Xenopus. Dev. Dyn. 231, 662-670.
Clark, J., Rocques, P. J., Crew, A. J., Gill, S., Shipley, J., Chan, A. M., Gusterson, B. A., and Cooper, C. S. (1994). Identification of novel genes, SYT and SSX, involved in the t(X; 18)(p11.2; q11.2) translocation found in human synovial sarcoma. Nat. Genet. 7, 502-508.
Crew, A. J., Clark, J., Fisher, C., Gill, S., Grimer, R., Chand, A., Shipley, J., Gusterson, B. A., and Cooper, C. S. (1995). Fusion of SYT to two genes, SSX1 and SSX2, encoding proteins with homology to the Kruppel-associated box in human synovial sarcoma. EMBO J. 14, 2333-2340.
de Bruijn, D. R., Baats, E., Zechner, U., de Leeuw, B., Balemans, M., Olde Weghuis, D., Hirning-Folz, U., and Geurts van Kessel, A. G. (1996). Isolation and characterization of the mouse homolog of SYT, a gene implicated in the development of human synovial sarcomas. Oncogene 13, 643-648.
de Bruijn, D. R., Kater-Baats, E., Eleveld, M., Merkx, G., and Geurts Van Kessel, A. (2001). Mapping and characterization of the mouse and human SS18 genes, two human SS18-like genes and a mouse Ss18 pseudogene. Cytogenet. Cell Genet. 92, 310-319.
de Leeuw, B., Suijkerbuijk, R. F., Olde Weghuis, D., Meloni, A. M., Stenman, G., Kindblom, L. G., Balemans, M., vandenBerg, E., Molenaar, W. M., Sandberg, A. A., et al. (1994). Distinct Xp11.2 breakpoint regions in synovial sarcoma revealed by metaphase and interphase FISH: Relationship to histologic subtypes. Cancer Genet. Cytogenet. 73, 89-94.
de Leeuw, B., Balemans, M., Olde Weghuis, D., and Geurts van Kessel, A. (1995). Identification of two alternative fusion genes, SYTSSX1 and SYT-SSX2, in t(X; 18)(p11.2; q11.2)-positive synovial sarcomas. Hum. Mol. Genet. 4, 1097-1099.
Detwiller, K. Y., Fernando, N. T., Segal, N. H., Ryeom, S. W., D'Amore, P. A., and Yoon, S. S. (2005). Analysis of hypoxia-related gene expression in sarcomas and effect of hypoxia on RNA interference of vascular endothelial cell growth factor A. Cancer Res. 65, 5881-5889.
dos Santos, N. R., de Bruijn, D. R., Kater-Baats, E., Otte, A. P., and van Kessel, A. G. (2000). Delineation of the protein domains responsible for SYT, SSX, and SYT-SSX nuclear localization. Exp. Cell Res. 256, 192-202.
dos Santos, N. R., de Bruijn, D. R., and van Kessel, A. G. (2001). Molecular mechanisms underlying human synovial sarcoma development. Genes Chromosomes Cancer 30, 1-14.
Engleka, K. A., Gitler, A. D., Zhang, M., Zhou, D. D., High, F. A., and Epstein, J. A. (2005). Insertion of Cre into the Pax3 locus creates a new allele of Splotch and identifies unexpected Pax3 derivatives. Dev. Biol. 280, 396-406.
Epstein, J. A. (2000). Pax3 and vertebrate development. Methods Mol. Biol. 137, 459-470.
Fisher, C. (1986). Synovial sarcoma: Ultrastructural and immunohistochemical features of epithelial differentiation in monophasic and biphasic tumors. Hum. Pathol. 17, 996-1008.
Frese, K. K., and Tuveson, D. A. (2007). Maximizing mouse cancer models. Nat Rev Cancer 7, 645-658.
Golub, T. R., Slonim, D. K., Tamayo, P., Huard, C., Gaasenbeek, M., Mesirov, J. P., Coller, H., Loh, M. L., Downing, J. R., Caligiuri, M. A., et al. (1999). Molecular classification of cancer: Class discovery and class prediction by gene expression monitoring. Science 286, 531-537.
Guillou, L., Coindre, J., Gallagher, G., Terrier, P., Gebhard, S., de Saint Aubain Somerhausen, N., Michels, J., Jundt, G., Vince, D. R., Collin, F., et al. (2001). Detection of the synovial sarcoma translocation t(X;18) (SYT;SSX) in paraffin-embedded tissues using reverse transcriptasepolymerase chain reaction: A reliable and powerful diagnostic tool for pathologists. A molecular analysis of 221 mesenchymal tumors fixed in different fixatives. Hum. Pathol. 32, 105-112.
Gure, A. O., Tureci, O., Sahin, U., Tsang, S., Scanlan, M. J., Jager, E., Knuth, A., Pfreundschuh, M., Old, L. J., and Chen, Y. T. (1997). SSX: A multigene family with several members transcribed in normal testis and human cancer. Int. J. Cancer 72, 965-971.
Haldar, M., Hancock, J. D., Coffin, C. M., Lessnick, S. L., and Capecchi, M. R. (2007). A conditional mouse model of synovial sarcoma: insights into a myogenic origin. Cancer Cell 11, 375-388.
Hayashi, S., and McMahon, A. P. (2002). Efficient recombination in diverse tissues by a tamoxifen-inducible form of Cre: A tool for temporally regulated gene activation/inactivation in the mouse. Dev. Biol. 244, 305-318.
Henderson, S. R., Guiliano, D., Presneau, N., McLean, S., Frow, R., Vujovic, S., Anderson, J., Sebire, N., Whelan, J., Athanasou, N., et al. (2005). A molecular map of mesenchymal tumors. Genome Biol. 6, R76.
Hibshoosh, H., and Lattes, R. (1997). Immunohistochemical and molecular genetic approaches to soft tissue tumor diagnosis: A primer. Semin. Oncol. 24, 515-525.
Hiraga, H., Nojima, T., Abe, S., Sawa, H., Yamashiro, K., Yamawaki, S., Kaneda, K., and Nagashima, K. (1998). Diagnosis of synovial sarcoma with the reverse transcriptase-polymerase chain reaction: Analyses of 84 soft tissue and bone tumors. Diagn. Mol. Pathol. 7, 102-110.
Jackson, R. J., Howell, M. T., and Kaminski, A. (1990). The novel mechanism of initiation of picornavirus RNA translation. Trends Biochem. Sci. 15, 477-483.
Jang, S. K., and Wimmer, E. (1990). Cap-independent translation of encephalomyocarditis virus RNA: Structural elements of the internal ribosomal entry site and involvement of a cellular 57-kD RNA-binding protein. Genes Dev. 4, 1560-1572.
Jostes, B., Walther, C., and Gruss, P. (1990). The murine paired box gene, Pax7, is expressed specifically during the development of the nervous and muscular system. Mech. Dev. 33, 27-37.
Kawai, A., Woodruff, J., Healey, J. H., Brennan, M. F., Antonescu, C. R., and Ladanyi, M. (1998). SYT-SSX gene fusion as a determinant of morphology and prognosis in synovial sarcoma. N. Engl. J. Med. 338, 153-160.
Keller, C., Arenkiel, B. R., Coffin, C. M., El-Bardeesy, N., DePinho, R. A., and Capecchi, M. R. (2004a). Alveolar rhabdomyosarcomas in conditional Pax3:Fkhr mice: Cooperativity of Ink4a/ARF and Trp53 loss of function. Genes Dev. 18, 2614-2626.
Keller, C., Hansen, M. S., Coffin, C. M., and Capecchi, M. R. (2004b). Pax3:Fkhr interferes with embryonic Pax3 and Pax7 function: Implications for alveolar rhabdomyosarcoma cell of origin. Genes Dev. 18, 2608-2613.
Ladanyi, M., and Bridge, J. A. (2000). Contribution of molecular genetic data to the classification of sarcomas. Hum. Pathol. 31, 532-538.
Ladanyi, M., Antonescu, C. R., Leung, D. H., Woodruff, J. M., Kawai, A., Healey, J. H., Brennan, M. F., Bridge, J. A., Neff, J. R., Barr, F. G., et al. (2002). Impact of SYT-SSX fusion type on the clinical behavior of synovial sarcoma: A multi-institutional retrospective study of 243 patients. Cancer Res. 62, 135-140.
Lim, F. L., Soulez, M., Koczan, D., Thiesen, H. J., and Knight, J. C. (1998). A KRAB-related domain and a novel transcription repression domain in proteins encoded by SSX genes that are disrupted in human sarcomas. Oncogene 17, 2013-2018.
Limon, J., Dal Cin, P., and Sandberg, A. A. (1986). Translocations involving the X chromosome in solid tumors: Presentation of two sarcomas with t(X;18)(q13;p 11). Cancer Genet. Cytogenet. 23, 87-91.
Nagai, M., Tanaka, S., Tsuda, M., Endo, S., Kato, H., Sonobe, H., Minami, A., Hiraga, H., Nishihara, H., Sawa, H., and Nagashima, K. (2001). Analysis of transforming activity of human synovial sarcomaassociated chimeric protein SYT-SSX1 bound to chromatin remodeling factor hBRM/hSNF2 alpha. Proc. Natl. Acad. Sci. USA 98, 3843-3848.
Nielsen, T. O., West, R. B., Linn, S. C., Alter, O., Knowling, M. A., O'Connell, J. X., Zhu, S., Fero, M., Sherlock, G., Pollack, J. R., et al. (2002). Molecular characterisation of soft tissue tumours: A gene expression study. Lancet 359, 1301-1307.
Oustanina, S., Hause, G., and Braun, T. (2004). Pax7 directs postnatal renewal and propagation of myogenic satellite cells but not their specification. EMBO J. 23, 3430-3439.
Panagopoulos, I., Mertens, F., Isaksson, M., Limon, J., Gustafson, P., Skytting, B., Akerman, M., Sciot, R., Dal Cin, P., Samson, I., et al. (2001). Clinical impact of molecular and cytogenetic findings in synovial sarcoma. Genes Chromosomes Cancer 31, 362-372.
Pelmus, M., Guillou, L., Hostein, I., Sierankowski, G., Lussan, C., and Coindre, J. M. (2002). Monophasic fibrous and poorly differentiated synovial sarcoma: Immunohistochemical reassessment of 60 t(X;18)(SYT-SSX)-positive cases. Am. J. Surg. Pathol. 26, 1434-1440.
Perani, M., Ingram, C. J., Cooper, C. S., Garrett, M. D., and Goodwin, G. H. (2003). Conserved SNH domain of the proto-oncoprotein SYT interacts with components of the human chromatin remodelling complexes, while the QPGY repeat domain forms homo-oligomers. Oncogene 22, 8156-8167.
Poteat, H. T., Corson, J. M., and Fletcher, J. A. (1995). Detection of chromosome 18 rearrangement in synovial sarcoma by fluorescence in situ hybridization. Cancer Genet. Cytogenet. 84, 76-81.
Pownall, M. E., Gustafsson, M. K., and Emerson, C. P., Jr. (2002). Myogenic regulatory factors and the specification of muscle progenitors in vertebrate embryos. Annu Rev. Cell Dev. Biol. 18, 747-783.
Renwick, P. J., Reeves, B. R., Dal Cin, P., Fletcher, C. D., Kempski, H., Sciot, R., Kazmierczak, B., Jani, K., Sonobe, H., and Knight, J. C. (1995). Two categories of synovial sarcoma defined by divergent chromosome translocation breakpoints in Xp11.2, with implications for the histologic sub-classification of synovial sarcoma. Cytogenet. Cell Genet. 70, 58-63.
Skytting, B., Nilsson, G., Brodin, B., Xie, Y., Lundeberg, J., Uhlen, M., and Larsson, O. (1999). A novel fusion gene, SYT-SSX4, in synovial sarcoma. J. Natl. Cancer Inst. 91, 974-975.
Smith, S., Reeves, B. R., Wong, L., and Fisher, C. (1987). A consistent chromosome translocation in synovial sarcoma. Cancer Genet. Cytogenet. 26, 179-180.
Smith, M. E., Fisher, C., Wilkinson, L. S., and Edwards, J. C. (1995). Synovial sarcoma lack synovial differentiation. Histopathology 26, 279-281.
Soriano, P. (1999). Generalized lacZ expression with the ROSA26 Cre reporter strain. Nat. Genet. 21, 70-71.
Srinivas, S., Watanabe, T., Lin, C. S., William, C. M., Tanabe, Y., Jessell, T. M., and Costantini, F. (2001). Cre reporter strains produced by targeted insertion of EYFP and ECFP into the ROSA26 locus. BMC Dev. Biol. 1, 4.
Sweet-Cordero, A., Mukherjee, S., Subramanian, A., You, H., Roix, J. J., Ladd-Acosta, C., Mesirov, J., Golub, T. R., and Jacks, T. (2005). An oncogenic KRAS2 expression signature identified by cross-species gene-expression analysis. Nat. Genet. 37, 48-55.
Tang, S. H., Silva, F. J., Tsark, W. M., and Mann, J. R. (2002). A Cre/loxPdeleter transgenic line in mouse strain 129S1/SvImJ. Genesis 32, 199-202.
Thaete, C., Brett, D., Monaghan, P., Whitehouse, S., Rennie, G., Rayner, E., Cooper, C. S., and Goodwin, G. (1999). Functional domains of the SYT and SYT-SSX synovial sarcoma translocation proteins and co-localization with the SNF protein BRM in the nucleus. Hum. Mol. Genet. 8, 585-591.
Torchia, E. C., Boyd, K., Rehg, J. E., Qu, C., and Baker, S. J. (2007). EWS/FLI-1 induces rapid onset of myeloid/erythroid leukemia in mice. Mol Cell Biol 27, 7918-7934.
Weiss, S. W., and Goldblum, J. R. (2001). Enzinger and Weiss's Soft Tissue Tumors, Fourth Edition (St. Louis, Mo.:: Mosby, Inc.).
Willeke, F., Mechtersheimer, G., Schwarzbach, M., Weitz, J., Zimmer, D., Lehnert, T., Herfarth, C., von Knebel Doeberitz, M., and Ridder, R. (1998). Detection of SYT-SSX1/2 fusion transcripts by reverse transcriptase-polymerase chain reaction (RT-PCR) is a valuable diagnostic tool in synovial sarcoma. Eur. J. Cancer 34, 2087-2093.
Wu, S., Wu, Y., and Capecchi, M. R. (2006). Motoneurons and oligodendrocytes are sequentially generated from neural stem cells but do not appear to share common lineage-restricted progenitors in vivo. Development 133, 581-590.
Yang, K.; Lui, W.-O.; Xie, Y.; Zhang, A.; Skytting, B.; Mandahl, N.; Larsson, C.; Larsson, O.: Co-existence of SYT-SSX1 and SYT-SSX2 fusions in synovial sarcomas. Oncogene 21:4181-4190, 2002.
Zambrowicz, B. P., Imamoto, A., Fiering, S., Herzenberg, L. A., Kerr, W. G., and Soriano, P. (1997). Disruption of overlapping transcripts in the ROSA beta geo 26 gene trap strain leads to widespread expression of beta-galactosidase in mouse embryos and hematopoietic cells. Proc. Natl. Acad. Sci. USA 94, 3789-3794.

### G. SEQUENCES

```
1. SEQ ID NO: 1 - SYT-SSX2
atgtctgtggctttcgcggccccgaggcagcgaggcaagggggagatcactcccgctgcgattcagaagatg
ttggatgacaataaccatcttattcagtgtataatggactctcagaataaaggaaagacctcagagtgttct
cagtatcagcagatgttgcacacaaacttggtataccttgctacaatagcagattctaatcaaaatatgcag
tctcttttaccagcaccacccacacagaatatgcctatgggtcctggagggatgaatcagagcggccctccc
ccacctccacgctctcacaacatgccttcagatggaatggtaggtgggggtcctcctgcaccgcacatgcag
aaccagatgaacggccagatgcctgggcctaaccatatgcctatgcagggacctggacccaatcaactcaat
atgacaaacagttccatgaatatgccttcaagtagccatggatccatgggaggttacaaccattctgtgcca
tcatcacagagcatgccagtacagaatcagatgacaatgagtcagggacaaccaatgggaaactatggtccc
agaccaaatatgagtatgcagccaaaccaaggtccaatgatgcatcagcagcctccttctcagcaatacaat
atgccacagggaggcggacagcattaccaaggacagcagccacctatgggaatgatgggtcaagttaaccaa
ggcaatcatatgatgggtcagagacagattcctccctatagacctcctcaacagggcccaccacagcagtac
tcaggccaggaagactattacggggaccaatacagtcatggtggacaaggtcctccagaaggcatgaaccag
caatattaccctgatggtcataatgattacggttatcagcaaccgtcgtatcctgaacaaggctacgatagg
ccttatgaggattcctcacaacattactacgaaggaggaaattcacagtatggccaacagcaagatgcatac
cagggaccacctccacaacagggatatccaccccagcagcagcagtacccagggcagcaaggttacccagga
cagcagcagggctacggtccttcacagggtggtccaggtcctcagtatcctaactacccacagggacaaggt
cagcagtatggaggatatagaccaacacagcctggaccaccacagccaccccagcagaggccttatggatat
gaccagatcatgcccaagaagccagcagaggaaggaaatgattcggaggaagtgccagaagcatctggccca
caaaatgatgggaaagagctgtgccccccgggaaaaccaactacctctgagaagattcacgagagatctgga
cccaaaaggggggaacatgcctggacccacagactgcgtgagagaaaacagctggtgatttatgaagagatc
agcgaccctgaggaagatgacgagtaa
2. SEQ ID NO: 2 - SYT-SSX2
MSVAFAAPRQRGKGEITPAAIQKMLDDNNHLIQCIMDSQNKGKTSECSQYQQMLHTNLVYLATIADSNQNMQ
SLLPAPPTQNMPMGPGGMNQSGPPPPPRSHNMPSDGMVGGGPPAPHMQNQMNGQMPGPNHMPMQGPGPNQLN
MTNSSMNMPSSSHGSMGGYNHSVPSSQSMPVQNQMTMSQGQPMGNYGPRPNMSMQPNQGPMMHQQPPSQQYN
MPQGGGQHYQGQQPPMGMMGQVNQGNHMMGQRQIPPYRPPQQGPPQQYSGQEDYYGDQYSHGGQGPPEGMNQ
QYYPDGHNDYGYQQPSYPEQGYDRPYEDSSQHYYEGGNSQYGQQQDAYQGPPPQQGYPPQQQQYPGQQGYPG
QQQGYGPSQGGPGPQYPNYPQGQGQQYGGYRPTQPGPPQPPQQRPYGYDQIMPKKPAEEGNDSEEVPEASGP
QNDGKELCPPGKPTTSEKIHERSGPKRGEHAWTHRLRERKQLVIYEEISDPEEDDE
3. SEQ ID NO: 3 - SYT cDNA (NM_001007559)
      1 gagaggccgg cgtctctccc ccagtttgcc gttcacccgg agcgctcggg acttgccgat
    61 agtggtgacg gcggcaacat gtctgtggct ttcgcggccc cgaggcagcg aggcaagggg
  121 gagatcactc ccgctgcgat tcagaagatg ttggatgaca ataaccatct tattcagtgt
  181 ataatggact ctcagaataa aggaaagacc tcagagtgtt ctcagtatca gcagatgttg
  241 cacacaaact tggtatacct tgctacaata gcagattcta atcaaaatat gcagtctctt
  301 ttaccagcac cacccacaca gaatatgcct atgggtcctg gagggatgaa tcagagcggc
  361 cctcccccac ctccacgctc tcacaacatg ccttcagatg gaatggtagg tgggggtcct
  421 cctgcaccgc acatgcagaa ccagatgaac ggccagatgc ctgggcctaa ccatatgcct
  481 atgcagggac ctggacccaa tcaactcaat atgacaaaca gttccatgaa tatgccttca
  541 agtagccatg gatccatggg aggttacaac cattctgtgc catcatcaca gagcatgcca
  601 gtacagaatc agatgacaat gagtcaggga caaccaatgg gaaactatgg tcccagacca
  661 aatatgagta tgcagccaaa ccaaggtcca atgatgcatc agcagcctcc ttctcagcaa
  721 tacaatatgc cacagggagg cggacagcat taccaaggac agcagccacc tatgggaatg
  781 atgggtcaag ttaaccaagg caatcatatg atgggtcaga gacagattcc tccctataga
  841 cctcctcaac agggcccacc acagcagtac tcaggccagg aagactatta cggggaccaa
  901 tacagtcatg gtggacaagg tcctccagaa ggcatgaacc agcaatatta ccctgatggt
  961 cataatgatt acggttatca gcaaccgtcg tatcctgaac aaggctacga taggccttat
1021 gaggattcct cacaacatta ctacgaagga ggaaattcac agtatggcca acagcaagat
1081 gcataccagg gaccacctcc acaacaggga tatccacccc agcagcagca gtacccaggg
1141 cagcaaggtt acccaggaca gcagcagggc tacggtcctt cacagggtgg tccaggtcct
1201 cagtatccta actacccaca gggacaaggt cagcagtatg gaggatatag accaacacag
1261 cctggaccac cacagccacc ccagcagagg ccttatggat atgaccaggg acagtatgga
1321 aattaccagc agtgaaaaag tacttacatt ccagtagcca gtatctatta gcagccatat
1381 tgtcacctca gcactgtgga cacctccctg tgaagagatc cttccattcc atctagtttt
1441 tggaaaaacc ttgtggataa gtggctgttt catcagtaag cagcctttgt ggtttagtta
1501 taaaaggctt tagtagctca aaaatactct tgatttcaca tttctactct agatggcaac
1561 attggacaga aaatgcaatg acataaccaa tttgtaatga ttttggaact gtgtttcaaa
1621 tggactgtta cagactgaaa ggtgtgaaca gctttgtatg tttatgaagg gtaagggaat
1681 ttaatacttt tccacagatt tttttgtaag gggaagaggg aaatgtacac tttttacagc
1741 agcaatattt tgtatattat gtttatttca tgtggtgaat atgcaaggcg gtacactacg
1801 cactggacag catcagaaat cctctgttaa tgtggactgg agcatggtag atgcttgatt
1861 gttttggtct caaaatggtg tgctataaag ataaaggtga ggggaagaca aagcacacca
1921 tatgtccact gttctgttct catagaggaa attcaaatcc cttttatcta ttagataatc
1981 aagggcactg tgatacagtt ttgagtaaaa agacattttt taaaagcctt ccagttttgt
2041 ggattaaacc tttttataaa gatcatttat aatactgttt taaaatgtga ggcaataaga
2101 attactttgt gttggatctg aggaggcttt ggtaaaacag tttcatctaa atgaaagtgg
2161 taatcctctt ctaaaatagc aataactgaa aatgaaagtg ttaattttac cttgtttgag
2221 ttatcaggga acttagtaag taatatcaaa gcattttata aatgatatca aagaagagtc
2281 aacattgatc cagtcatttt attttgtaat attgagggat aattggttat taaactgaat
2341 agttcaggag actttacaaa cctttgtttc aactttctta tctggaaata atatcattta
2401 taaagggaca cttttatgtt tttccctttt ttatgttggt tgatataaca caaagagata
2461 tttaggaaaa tgcttattga tgaggtttat tctatctgtt tttaaagcac cgaggttgca
2521 ttctagataa ccttgtttat tagcatggca tattttaatc attatttgag actgtcctgt
2581 gcctgattat tttagctaaa ttcagggaga ttgcgtgggg caggaaagca tgcattgaaa
2641 aatttctaac cacggttatt taagcataat ctgaaaacat ctagcccaaa ggtaagttgc
2701 tattttcatc acagttgcct atgcccaggg aataagatgt attctttata attgaattgg
2761 tttttcccac gtctaactgg aaacaaaaca gaaggggcgt cataaatttg aataagcaga
2821 acatactgtt ctcaacatac tgtaatcaaa aggaggaatt tcagtgggtc tctgtgtgtg
2881 tatgagagag agagtgtgtg tttgtgtgtt tcaaggtcag aacaggtttt tttgtttttg
2941 ttttttgttc tttgtttttt tttttgagat ggagtcttgc tcttgtcgcc caggctggag
3001 tgcagtggcg caatctcagc tcactgcaac ctccgcctcc caggttcaag cagttctcct
3061 gcctcagcct cctgagtagc tgggatgaca ggcacccgcc accacaccca gctaattttt
3121 gtacttttag tagagacgag gtttcgccat gttggccagg ctggtctcga actcctgacc
3181 tcaggtgatc cacccgcctc ggccttccaa agtgctggga ttacaggcgt gagccaccgt
3241 gcctggccag aataggtttt ttctttcaac ttgatcagta gaaaatggac atcaagtttg
3301 aacagataaa tcatggacag ccttattgtg attgaaatgc ttgtaggttc tgtgccaatt
3361 ttccaccact gtgtactttg ttgctattta aaactgtatc aactctaacg gaagaataaa
3421 ttatttgtga ttttaaaaaa
4. SEQ ID NO: 4 - SYT coding sequence
atgtctgtggctttcgcggccccgaggcagcgaggcaagggggagatcactcccgctgcgattcagaagatg
ttggatgacaataaccatcttattcagtgtataatggactctcagaataaaggaaagacctcagagtgttct
cagtatcagcagatgttgcacacaaacttggtataccttgctacaatagcagattctaatcaaaatatgcag
tctcttttaccagcaccacccacacagaatatgcctatgggtcctggagggatgaatcagagcggccctccc
ccacctccacgctctcacaacatgccttcagatggaatggtaggtgggggtcctcctgcaccgcacatgcag
aaccagatgaacggccagatgcctgggcctaaccatatgcctatgcagggacctggacccaatcaactcaat
atgacaaacagttccatgaatatgccttcaagtagccatggatccatgggaggttacaaccattctgtgcca
tcatcacagagcatgccagtacagaatcagatgacaatgagtcagggacaaccaatgggaaactatggtccc
agaccaaatatgagtatgcagccaaaccaaggtccaatgatgcatcagcagcctccttctcagcaatacaat
atgccacagggaggcggacagcattaccaaggacagcagccacctatgggaatgatgggtcaagttaaccaa
ggcaatcatatgatgggtcagagacagattcctccctatagacctcctcaacagggcccaccacagcagtac
tcaggccaggaagactattacggggaccaatacagtcatggtggacaaggtcctccagaaggcatgaaccag
caatattaccctgatggtcataatgattacggttatcagcaaccgtcgtatcctgaacaaggctacgatagg
ccttatgaggattcctcacaacattactacgaaggaggaaattcacagtatggccaacagcaagatgcatac
cagggaccacctccacaacagggatatccaccccagcagcagcagtacccagggcagcaaggttacccagga
cagcagcagggctacggtccttcacagggtggtccaggtcctcagtatcctaactacccacagggacaaggt
cagcagtatggaggatatagaccaacacagcctggaccaccacagccaccccagcagaggccttatggatat
gaccagggacagtatggaaattaccagcagtga
5. SEQ ID NO: 5 - SYT (NM_001007559)
MSVAFAAPRQRGKGEITPAAIQKMLDDNNHLIQCIMDSQNKGKTSECSQYQQMLHTNLVYLATIADSNQNMQ
SLLPAPPTQNMPMGPGGMNQSGPPPPPRSHNMPSDGMVGGGPPAPHMQNQMNGQMPGPNHMPMQGPGPNQLN
MTNSSMNMPSSSHGSMGGYNHSVPSSQSMPVQNQMTMSQGQPMGNYGPRPNMSMQPNQGPMMHQQPPSQQYN
MPQGGGQHYQGQQPPMGMMGQVNQGNHMMGQRQIPPYRPPQQGPPQQYSGQEDYYGDQYSHGGQGPPEGMNQ
QYYPDGHNDYGYQQPSYPEQGYDRPYEDSSQHYYEGGNSQYGQQQDAYQGPPPQQGYPPQQQQYPGQQGYPG
QQQGYGPSQGGPGPQYPNYPQGQGQQYGGYRPTQPGPPQPPQQRPYGYDQGQYGNYQQ
6. SEQ ID NO: 6 - SYT N-Terminal (SNH) domain
cgaggcaagggggagatcactcccgctgcgattcagaagatgttggatgacaataaccatcttattcagtgt
ataatggactctcagaataaaggaaagacctcagagtgttctcagtatcagcagatgttgcacacaaacttg
gtataccttgctacaatagcagattctaatcaaaatatgcagtctcttttacca
7. SEQ ID NO: 7 - SNH domain
RGKGEITPAAIQKMLDDNNHLIQCIMDSQNKGKTSECSQYQQMLHTNLVYLATIADSNQNMQSLLP
8. SEQ ID NO: 8 - QPGY domain (547-711 dispensible)
Cagggacaaccaatgggaaactatggtcccagaccaaatatgagtatgcagccaaaccaaggtccaatgatg
catcagcagcctccttctcagcaatacaatatgccacagggaggcggacagcattaccaaggacagcagcca
cctatgggaatgatgggtcaagttaaccaaggcaatcatatgatgggtcagagacagattcctccctataga
cctcctcaacagggcccaccacagcagtactcaggccaggaagactattacggggaccaatacagtcatggt
ggacaaggtcctccagaaggcatgaaccagcaatattaccctgatggtcataatgattacggttatcagcaa
ccgtcgtatcctgaacaaggctacgataggccttatgaggattcctcacaacattactacgaaggaggaaat
tcacagtatggccaacagcaagatgcataccagggaccacctccacaacagggatatccaccccagcagcag
cagtacccagggcagcaaggttacccaggacagcagcagggctacggtccttcacagggtggtccaggtcct
cagtatcctaactacccacagggacaaggtcagcagtatggaggatatagaccaacacagcctggaccacca
cagccaccccagcagaggccttatggatatgaccagggacagtatggaaattaccagcagtga
9. SEQ ID NO: 9 - QPGY domain (183-236 dispensible)
QGQPMGNYGPRPNMSMQPNQGPMMHQQPPSQQYNMPQGGGQHYQGQQPPMGMMGQVNQGNHMMGQRQIPPYR
PPQQGPPQQYSGQEDYYGDQYSHGGQGPPEGMNQQYYPDGHNDYGYQQPSYPEQGYDRPYEDSSQHYYEGGN
SQYGQQQDAYQGPPPQQGYPPQQQQYPGQQGYPGQQQGYGPSQGGPGPQYPNYPQGQGQQYGGYRPTQPGPP
QPPQQRPYGYDQGQYGNYQQ
10. SEQ ID NO: 10 - QPGY domain 1-182
QGQPMGNYGPRPNMSMQPNQGPMMHQQPPSQQYNMPQGGGQHYQGQQPPMGMMGQVNQGNHMMGQRQIPPYR
PPQQGPPQQYSGQEDYYGDQYSHGGQGPPEGMNQQYYPDGHNDYGYQQPSYPEQGYDRPYEDSSQHYYEGGN
SQYGQQQDAYQGPPPQQGYPPQQQQYPGQQGYPGQQQG
11. SEQ ID NO: 11 - SSX2 cDNA (NM_175698)
      1 gcatgctctg actttctctc tctttcgatt cttccatact cagagtacgc acggtctgat
    61 tttctctttg gattcttcca aaatcagagt cagactgctc ccggtgccat gaacggagac
  121 gacgcctttg caaggagacc cacggttggt gctcaaatac cagagaagat ccaaaaggcc
  181 ttcgatgata ttgccaaata cttctctaag gaagagtggg aaaagatgaa agcctcggag
  241 aaaatcttct atgtgtatat gaagagaaag tatgaggcta tgactaaact aggtttcaag
  301 gccaccctcc cacctttcat gtgtaataaa cgggccgaag acttccaggg gaatgatttg
  361 gataatgacc ctaaccgtgg gaatcaggtt gaacgtcctc agatgacttt cggcaggctc
  421 cagggaatct ccccgaagat catgcccaag aagccagcag aggaaggaaa tgattcggag
  481 gaagtgccag aagcatctgg cccacaaaat gatgggaaag agctgtgccc cccgggaaaa
  541 ccaactacct ctgagaagat tcacgagaga tctggaccca aaagggggga acatgcctgg
  601 acccacagac tgcgtgagag aaaacagctg gtgatttatg aagagatcag cgaccctgag
  661 gaagatgacg agtaactccc ctcagggata cgacacatgc ccatgatgag aagcagaacg
  721 tggtgacctt tcacgaacat gggcatggct gcggacccct cgtcatcagg tgcatagcaa
  781 gtgaaagcaa gtgttcacaa cagtgaaaag ttgagcgtca tttttcttag tgtgccaaga
  841 gttcgatgtt agcgtttacg ttgtattttc ttacactgtg tcattctgtt agatactaac
  901 attttcattg atgagcaaga catacttaat gcatattttg gtttgtgtat ccatgcacct
  961 accttagaaa acaagtattg tcggttacct ctgcatggaa cagcattacc ctcctctctc
1021 cccagatgtg actactgagg gcagttctga gtgtttaatt tcagattttt tcctctgcat
1081 ttacacacac acgcacacaa accacaccac acacacacac acacacacac acacacacac
1141 acacacacac caagtaccag tataagcatc tgccatctgc ttttcccatt gccatgcgtc
1201 ctggtcaagc tcccctcact ctgtttcctg gtcagcatgt actcccctca tccgattccc
1261 ctgtagcagt cactgacagt taataaacct ttgcaaacgt tcaaaaaaaa aaaaaaaaaa
1321 aa
12. SEQ ID NO: 12 - SSX2 coding sequence (1-330 dispensible)
atgaacggagacgacgcctttgcaaggagacccacggttggtgctcaaataccagagaagatccaaaaggcc
ttcgatgatattgccaaatacttctctaaggaagagtgggaaaagatgaaagcctcggagaaaatcttctat
gtgtatatgaagagaaagtatgaggctatgactaaactaggtttcaaggccaccctcccacctttcatgtgt
aataaacgggccgaagacttccaggggaatgatttggataatgaccctaaccgtgggaatcaggttgaacgt
cctcagatgactttcggcaggctccagggaatctccccgaagatcatgcccaagaagccagcagaggaagga
aatgattcggaggaagtgccagaagcatctggcccacaaaatgatgggaaagagctgtgccccccgggaaaa
ccaactacctctgagaagattcacgagagatctggacccaaaaggggggaacatgcctggacccacagactg
cgtgagagaaaacagctggtgatttatgaagagatcagcgaccctgaggaagatgacgagtaa
13. SEQ ID NO: 13 - SSX2 (NM_175698)
MNGDDAFARRPTVGAQIPEKIQKAFDDIAKYFSKEEWEKMKASEKIFYVYMKRKYEAMTKLGFKATLPPFMC
NKRAEDFQGNDLDNDPNRGNQVERPQMTFGRLQGISPKIMPKKPAEEGNDSEEVPEASGPQNDGKELCPPGK
PTTSEKIHERSGPKRGEHAWTHRLRERKQLVIYEEISDPEEDDE
14. SEQ ID NO: 14 - SSXRD domain
ggacccaaaaggggggaacatgcctggacccacagactgcgtgagagaaaacagctggtgatttatgaagag
atcagcgaccctgaggaagatgacgagtaa
15. SEQ ID NO: 15 - SSXRD domain
IMPKKPAEEGNDSEEVPEASGPQNDGKELCPPGKPTTSEKIHERSGPKRGEHAWTHRLRERKQLVIYEEISD
PEEDDE
16. SEQ ID NO: 16 - loxP
ataacttcgtataatgtatgctatacgaagttat
17. SEQ ID NO:17
tggatgggcggcaacatgtctgtgg
18. SEQ ID NO: 18
gtgaggggggcttgaccaggacgca
19. SEQ ID NO: 19
gttatcagtaagggagctgcagtgg
20. SEQ ID NO: 20
aagaccgcgaagagtttgtcctc
21. SEQ ID NO: 21
ggcggatcacaagcaataataacc
22. SEQ ID NO: 22
ggatttccgtctctggtgtagc
23. SEQ ID NO: 23
accattgcccctgttcactatc
24. SEQ ID NO: 24
accctccagctccagacttatc
25. SEQ ID NO: 25
ccctgtaatggattccaagctg
26. SEQ ID NO: 26
aaagacccctaggaatgctc
```

1. A non-human animal model of synovial sarcoma, wherein one or more cells of the animal express recombinant SYT-SSX fusion polypeptide.

2. The non-human mammal of claim 1, wherein SYT-SSX fusion polypeptide is SYT-SSX1, SYT-SSX2, or SYT-SSX4.

3. The non-human mammal of claim 1, wherein the SYT-SSX fusion polypeptide has at least 95% sequence identity to the amino acid sequence SEQ ID NO:2.

4. The non-human mammal of claim 1, wherein the SYT-SSX fusion polypeptide comprises a SYT N-terminal domain.

5. The non-human mammal of claim 4, wherein the SYT N-terminal domain has at least 95% sequence identity to the amino acid sequence SEQ ID NO:7.

6. The non-human mammal of claim 1, wherein the SYT-SSX fusion polypeptide comprises a QPGY domain.

7. The non-human mammal of claim 4, wherein the QPGY domain has at least 95% sequence identity to the amino acid sequence SEQ ID NO:10.

8. The non-human mammal of claim 1, wherein the SYT-SSX fusion polypeptide comprises an SSX Repression domain (SSXRD domain).

9. The non-human mammal of claim 4, wherein the SSXRD domain has at least 95% sequence identity to the amino acid sequence SEQ ID NO:15.

10. The non-human mammal of claim 1, wherein the one or more cells of the mammal comprise a nucleic acid encoding a SYT-SSX fusion polypeptide operably linked to a myogenic-specific expression control sequence.

11. The non-human mammal of claim 10, wherein the myogenic-specific expression control sequence is the Myf5 promoter.

12. The non-human mammal of claim 1, wherein the one or more cells of the mammal comprise a first and second polynucleotide, wherein
(e) the first polynucleotide comprises a nucleic acid sequence encoding a SYT-SSX fusion polypeptide operably linked to a first expression control sequence and a transcriptional termination signal, wherein the transcription termination signal substantially prevents expression of the SYT-SSX fusion polypeptide, and
(f) the second polynucleotide comprises a nucleic acid encoding a transactivator polypeptide operably linked to a second expression control sequence,
wherein expression of the transactivator polypeptide abolishes the effect of the transcription termination signal to substantially prevent expression of the SYT-SSX fusion polypeptide, wherein the non-human animal comprises synovial sarcomas.

13. The non-human mammal of claim 12, wherein the first expression control sequence is 5′ to the transcriptional termination signal, and wherein transcriptional termination signal is 5′ to the nucleic acid sequence encoding a SYT-SSX fusion polypeptide.

14. The non-human mammal of claim 12, wherein the transactivator polypeptide excises the transcriptional termination signal from the first polynucleotide.

15. The non-human mammal of claim 12, wherein the first expression control sequence is ROSA26.

16. The non-human mammal of claim 12, wherein the second expression control sequence is a myogenic-specific expression control sequence.

17. The non-human mammal of claim 16, wherein the myogenic-specific expression control sequence is the Myf5 promoter, MyoD promoter, or MyoG promoter.

18. The non-human mammal of claim 17, wherein the second nucleic acid comprises a nucleic acid encoding Myf5 that is 5′ to an internal ribosome entry site (IRES) that is 5′ to the nucleic acid encoding the transactivator polypeptide.

19. The non-human mammal of claim 12, wherein the transactivator polypeptide is Cre recombinase, wherein the transcription termination signal is flanked by LoxP.

20. The non-human mammal of claim 12, wherein the transactivator polypeptide is inducible.

21. The non-human mammal of claim 20, wherein the transactivator polypeptide is a fusion protein comprising Cre recombinase and an estrogen receptor, wherein the transcription termination signal is flanked by LoxP, wherein the inducer is tamoxifin.

22. The non-human mammal of claim of claim 12, wherein the transcription termination signal is a nucleic acid comprising a polyadenylation signal (PolyA).

23. The non-human mammal of claim 22, wherein nucleic acid comprising the PolyA encodes a neomycin resistance coding sequence.

24. The non-human mammal of claim 12, wherein the transcription termination signal is a stop codon.

25. The non-human mammal of claim 12, wherein the second polynucleotide further comprises a nucleic acid encoding a detection marker.

26. The non-human mammal of claim 25, wherein the detection marker is a fluorescent protein.

27. A non-human mammal, wherein one or more cells of the mammal comprise a nucleic acid sequence encoding a SYT-SSX fusion polypeptide operably linked to a first expression control sequence and a transcriptional termination signal, wherein the transcription termination signal substantially prevents expression of the SYT-SSX fusion polypeptide, wherein expression of Cre recombinase by the cell alters the transcription termination signal whereby the SYT-SSX fusion polypeptide is expressed.

28. The non-human mammal of claim 1, wherein the non-human mammal is not immunocompromised.

29. The non-human mammal of claim 1, wherein the non-human mammal is a rodent.

30. A cell comprising a first and second polynucleotide, wherein
(g) the first polynucleotide comprises a nucleic acid sequence encoding a SYT-SSX fusion polypeptide operably linked to a first expression control sequence and a transcriptional termination signal, wherein the transcription termination signal substantially prevents expression of the SYT-SSX fusion polypeptide, and
(h) the second polynucleotide comprises a nucleic acid encoding a transactivator polypeptide operably linked to a second expression control sequence, wherein expression of the transactivator polypeptide abolishes the effect of the transcription termination signal to substantially prevent expression of the SYT-SSX fusion polypeptide.

31. The cell of claim 30, wherein SYT-SSX fusion polypeptide is SYT-SSX1, SYT-SSX2, or SYT-SSX4.

32. The cell of claim 31, wherein the SYT-SSX fusion polypeptide has at least 95% sequence identity to the amino acid sequence SEQ ID NO:2.

33. The cell of claim 30, wherein the SYT-SSX fusion polypeptide comprises a SYT N-terminal domain.

34. The cell of claim 33, wherein the SYT N-terminal domain has at least 95% sequence identity to the amino acid sequence SEQ ID NO:7.

35. The cell of claim 30, wherein the SYT-SSX fusion polypeptide comprises a QPGY domain.

36. The cell of claim 35, wherein the QPGY domain has at least 95% sequence identity to the amino acid sequence SEQ ID NO:10.

37. The cell of claim 30, wherein the SYT-SSX fusion polypeptide comprises an SSX Repression domain (SSXRD domain).

38. The cell of claim 37, wherein the SSXRD domain has at least 95% sequence identity to the amino acid sequence SEQ ID NO:15.

39. The cell of claim 30, wherein the first expression control sequence is 5′ to the transcriptional termination signal, and wherein transcriptional termination signal is 5′ to the nucleic acid sequence encoding a SYT-SSX fusion polypeptide.

40. The cell of claim 30, wherein the transactivator polypeptide excises the transcriptional termination signal from the first polynucleotide.

41. The cell of claim 30, wherein the first expression control sequence is ROSA26.

42. The cell of claim 30, wherein the second expression control sequence is a myogenic-specific expression control sequence.

43. The cell of claim 42, wherein the myogenic-specific expression control sequence is the Myf5 promoter, MyoD promoter, or MyoG promoter.

44. The cell of claim 43, wherein the second nucleic acid comprises a nucleic acid encoding Myf5 that is 5′ to an internal ribosome entry site (IRES) that is 5′ to the nucleic acid encoding the transactivator polypeptide.

45. The cell of claim 30, wherein the transactivator polypeptide is Cre recombinase, wherein the transcription termination signal is flanked by LoxP.

46. The cell of claim 30, wherein the transactivator polypeptide is inducible.

47. The cell of claim 46, wherein the transactivator polypeptide is a fusion protein comprising Cre recombinase and an estrogen receptor, wherein the transcription termination signal is flanked by LoxP, wherein the inducer is tamoxifin.

48. The cell of claim 30, wherein the transcription termination signal is a nucleic acid comprising a polyadenylation signal (PolyA).

49. The cell of claim 48, wherein nucleic acid comprising the PolyA encodes a neomycin resistance coding sequence.

50. The cell of claim 30, wherein the transcription termination signal is a stop codon.

51. The cell of claim 30, wherein the second polynucleotide further comprises a nucleic acid encoding a detection marker.

52. The cell of claim 51, wherein the detection marker is a fluorescent protein.


SEQUENCE LISTING (26 sequences)

SEQ ID NO: 1 - DNA, 1467 bp, Artificial Sequence
  Description of Artificial Sequence Note = Synthetic Construct
atgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggtcataatgattacggttatcagcaaccgtcgtatcctgaacaaggctacgataggccttatgaggattcctcacaacattactacgaaggaggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaa

SEQ ID NO: 2 - protein, 488 aa, Artificial Sequence
  Description of Artificial Sequence Note = Synthetic Construct
MSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGHNDYGYQQPSYPEQGYDRPYEDSSQHYYEGGNSQYGQQQDAYQGPPPQQGYPPQQQQYPGQQGYPGQQQGYGPSQGGPGPQYPNYPQGQGQQYGGYRPTQPGPPQPPQQRPYGYDQIMPKKPAEEGNDSEEVPEASGPQNDGKELCPPGKPTTSEKIHERSGPKRGEHAWTHRLRERKQLVIYEEISDPEEDDE

SEQ ID NO: 3 - DNA, 3440 bp, Artificial Sequence
  Description of Artificial Sequence Note = Synthetic Construct
gagaggccggcgtctctcccccagtttgccgttcacccggagcgctcgggacttgccgatagtggtgacggcggcaacatgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggtcataatgattacggttatcagcaaccgtcgtatcctgaacaaggctacgataggccttatgaggattcctcacaacattactacgaaggaggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcagtgaaaaagtacttacattccagtagccagtatctattagcagccatattgtcacctcagcactgtggacacctccctgtgaagagatccttccattccatctagtttttggaaaaaccttgtggataagtggctgtttcatcagtaagcagcctttgtggtttagttataaaaggctttagtagctcaaaaatactcttgatttcacatttctactctagatggcaacattggacagaaaatgcaatgacataaccaatttgtaatgattttggaactgtgtttcaaatggactgttacagactgaaaggtgtgaacagctttgtatgtttatgaagggtaagggaatttaatacttttccacagatttttttgtaaggggaagagggaaatgtacactttttacagcagcaatattttgtatattatgtttatttcatgtggtgaatatgcaaggcggtacactacgcactggacagcatcagaaatcctctgttaatgtggactggagcatggtagatgcttgattgttttggtctcaaaatggtgtgctataaagataaaggtgaggggaagacaaagcacaccatatgtccactgttctgttctcatagaggaaattcaaatcccttttatctattagataatcaagggcactgtgatacagttttgagtaaaaagacattttttaaaagccttccagttttgtggattaaacctttttataaagatcatttataatactgttttaaaatgtgaggcaataagaattactttgtgttggatctgaggaggctttggtaaaacagtttcatctaaatgaaagtggtaatcctcttctaaaatagcaataactgaaaatgaaagtgttaattttaccttgtttgagttatcagggaacttagtaagtaatatcaaagcattttataaatgatatcaaagaagagtcaacattgatccagtcattttattttgtaatattgagggataattggttattaaactgaatagttcaggagactttacaaacctttgtttcaactttcttatctggaaataatatcatttataaagggacacttttatgtttttcccttttttatgttggttgatataacacaaagagatatttaggaaaatgcttattgatgaggtttattctatctgtttttaaagcaccgaggttgcattctagataaccttgtttattagcatggcatattttaatcattatttgagactgtcctgtgcctgattattttagctaaattcagggagattgcgtggggcaggaaagcatgcattgaaaaatttctaaccacggttatttaagcataatctgaaaacatctagcccaaaggtaagttgctattttcatcacagttgcctatgcccagggaataagatgtattctttataattgaattggtttttcccacgtctaactggaaacaaaacagaaggggcgtcataaatttgaataagcagaacatactgttctcaacatactgtaatcaaaaggaggaatttcagtgggtctctgtgtgtgtatgagagagagagtgtgtgtttgtgtgtttcaaggtcagaacaggtttttttgtttttgttttttgttctttgtttttttttttgagatggagtcttgctcttgtcgcccaggctggagtgcagtggcgcaatctcagctcactgcaacctccgcctcccaggttcaagcagttctcctgcctcagcctcctgagtagctgggatgacaggcacccgccaccacacccagctaatttttgtacttttagtagagacgaggtttcgccatgttggccaggctggtctcgaactcctgacctcaggtgatccacccgcctcggccttccaaagtgctgggattacaggcgtgagccaccgtgcctggccagaataggttttttctttcaacttgatcagtagaaaatggacatcaagtttgaacagataaatcatggacagccttattgtgattgaaatgcttgtaggttctgtgccaattttccaccactgtgtactttgttgctatttaaaactgtatcaactctaacggaagaataaattatttgtgattttaaaaaa

SEQ ID NO: 4 - DNA, 1257 bp, Artificial Sequence
  Description of Artificial Sequence Note = Synthetic Construct
atgtctgtggctttcgcggccccgaggcagcgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttaccagcaccacccacacagaatatgcctatgggtcctggagggatgaatcagagcggccctcccccacctccacgctctcacaacatgccttcagatggaatggtaggtgggggtcctcctgcaccgcacatgcagaaccagatgaacggccagatgcctgggcctaaccatatgcctatgcagggacctggacccaatcaactcaatatgacaaacagttccatgaatatgccttcaagtagccatggatccatgggaggttacaaccattctgtgccatcatcacagagcatgccagtacagaatcagatgacaatgagtcagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggtcataatgattacggttatcagcaaccgtcgtatcctgaacaaggctacgataggccttatgaggattcctcacaacattactacgaaggaggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcagtga

SEQ ID NO: 5 - protein, 418 aa, Artificial Sequence
  Description of Artificial Sequence  Note = Synthetic Construct
MSVAFAAPRQRGKGEITPAAIQKMLDDNNHLIQCIMDSQNKGKTSECSQYQQMLHTNLVYLATIADSNQNMQSLLPAPPTQNMPMGPGGMNQSGPPPPPRSHNMPSDGMVGGGPPAPHMQNQMNGQMPGPNHMPMQGPGPNQLNMTNSSMNMPSSSHGSMGGYNHSVPSSQSMPVQNQMTMSQGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGHNDYGYQQPSYPEQGYDRPYEDSSQHYYEGGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 6 - DNA, 198 bp, Artificial Sequence
  Description of Artificial Sequence Note = Synthetic Construct
cgaggcaagggggagatcactcccgctgcgattcagaagatgttggatgacaataaccatcttattcagtgtataatggactctcagaataaaggaaagacctcagagtgttctcagtatcagcagatgttgcacacaaacttggtataccttgctacaatagcagattctaatcaaaatatgcagtctcttttacca

SEQ ID NO: 7 - protein, 66 aa, Artificial Sequence
  Description of Artificial Sequence Note = Synthetic Construct
RGKGEITPAAIQKMLDDNNHLIQCIMDSQNKGKTSECSQYQQMLHTNLVYLATIADSNQNMQSLLP

SEQ ID NO: 8 - DNA, 711 bp, Artificial Sequence
  Description of Artificial Sequence Note = Synthetic Construct
cagggacaaccaatgggaaactatggtcccagaccaaatatgagtatgcagccaaaccaaggtccaatgatgcatcagcagcctccttctcagcaatacaatatgccacagggaggcggacagcattaccaaggacagcagccacctatgggaatgatgggtcaagttaaccaaggcaatcatatgatgggtcagagacagattcctccctatagacctcctcaacagggcccaccacagcagtactcaggccaggaagactattacggggaccaatacagtcatggtggacaaggtcctccagaaggcatgaaccagcaatattaccctgatggtcataatgattacggttatcagcaaccgtcgtatcctgaacaaggctacgataggccttatgaggattcctcacaacattactacgaaggaggaaattcacagtatggccaacagcaagatgcataccagggaccacctccacaacagggatatccaccccagcagcagcagtacccagggcagcaaggttacccaggacagcagcagggctacggtccttcacagggtggtccaggtcctcagtatcctaactacccacagggacaaggtcagcagtatggaggatatagaccaacacagcctggaccaccacagccaccccagcagaggccttatggatatgaccagggacagtatggaaattaccagcagtga

SEQ ID NO: 9 - protein, 236 aa, Artificial Sequence
  Description of Artificial Sequence Note = Synthetic Construct
QGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGHNDYGYQQPSYPEQGYDRPYEDSSQHYYEGGNSQYGQQQDAYQGPPPQQGYPPQQQQYPGQQGYPGQQQGYGPSQGGPGPQYPNYPQGQGQQYGGYRPTQPGPPQPPQQRPYGYDQGQYGNYQQ

SEQ ID NO: 10 - protein, 182 aa, Artificial Sequence
  Description of Artificial Sequence Note = Synthetic Construct
QGQPMGNYGPRPNMSMQPNQGPMMHQQPPSQQYNMPQGGGQHYQGQQPPMGMMGQVNQGNHMMGQRQIPPYRPPQQGPPQQYSGQEDYYGDQYSHGGQGPPEGMNQQYYPDGHNDYGYQQPSYPEQGYDRPYEDSSQHYYEGGNSQYGQQQDAYQGPPPQQGYPPQQQQYPGQQGYPGQQQG

SEQ ID NO: 11 - DNA, 1322 bp, Artificial Sequence
  Description of Artificial Sequence Note = Synthetic Construct
gcatgctctgactttctctctctttcgattcttccatactcagagtacgcacggtctgattttctctttggattcttccaaaatcagagtcagactgctcccggtgccatgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaactcccctcagggatacgacacatgcccatgatgagaagcagaacgtggtgacctttcacgaacatgggcatggctgcggacccctcgtcatcaggtgcatagcaagtgaaagcaagtgttcacaacagtgaaaagttgagcgtcatttttcttagtgtgccaagagttcgatgttagcgtttacgttgtattttcttacactgtgtcattctgttagatactaacattttcattgatgagcaagacatacttaatgcatattttggtttgtgtatccatgcacctaccttagaaaacaagtattgtcggttacctctgcatggaacagcattaccctcctctctccccagatgtgactactgagggcagttctgagtgtttaatttcagattttttcctctgcatttacacacacacgcacacaaaccacaccacacacacacacacacacacacacacacacacacacacacaccaagtaccagtataagcatctgccatctgcttttcccattgccatgcgtcctggtcaagctcccctcactctgtttcctggtcagcatgtactcccctcatccgattcccctgtagcagtcactgacagttaataaacctttgcaaacgttcaaaaaaaaaaaaaaaaaaaa

SEQ ID NO: 12 - DNA, 567 bp, Artificial Sequence
  Description of Artificial Sequence Note = Synthetic Construct
atgaacggagacgacgcctttgcaaggagacccacggttggtgctcaaataccagagaagatccaaaaggccttcgatgatattgccaaatacttctctaaggaagagtgggaaaagatgaaagcctcggagaaaatcttctatgtgtatatgaagagaaagtatgaggctatgactaaactaggtttcaaggccaccctcccacctttcatgtgtaataaacgggccgaagacttccaggggaatgatttggataatgaccctaaccgtgggaatcaggttgaacgtcctcagatgactttcggcaggctccagggaatctccccgaagatcatgcccaagaagccagcagaggaaggaaatgattcggaggaagtgccagaagcatctggcccacaaaatgatgggaaagagctgtgccccccgggaaaaccaactacctctgagaagattcacgagagatctggacccaaaaggggggaacatgcctggacccacagactgcgtgagagaaaacagctggtgatttatgaagagatcagcgaccctgaggaagatgacgagtaa

SEQ ID NO: 13 - protein, 188 aa, Artificial Sequence
  Description of Artificial Sequence Note = Synthetic Construct
MNGDDAFARRPTVGAQIPEKIQKAFDDIAKYFSKEEWEKMKASEKIFYVYMKRKYEAMTKLGFKATLPPFMCNKRAEDFQGNDLDNDPNRGNQVERPQMTFGRLQGISPKIMPKKPAEEGNDSEEVPEASGPQNDGKELCPPGKPTTSEKIHERSGPKRGEHAWTHRLRERKQLVIYEEISDPEEDDE

SEQ ID NO: 14 - DNA, 102 bp, Artificial Sequence
  Description of Artificial Sequence Note = Synthetic Construct
ggacccaaaaggggggaacatgcctggacccacagactgcgtgagagaaaacagctggtgatttatgaagagatcagcgaccctgaggaagatgacgagtaa

SEQ ID NO: 15 - protein, 78 aa, Artificial Sequence
  Description of Artificial Sequence Note = Synthetic Construct
IMPKKPAEEGNDSEEVPEASGPQNDGKELCPPGKPTTSEKIHERSGPKRGEHAWTHRLRERKQLVIYEEISDPEEDDE

SEQ ID NO: 16 - DNA, 34 bp, Artificial Sequence
  Description of Artificial Sequence Note = Synthetic Construct
ataacttcgtataatgtatgctatacgaagttat

SEQ ID NO: 17 - DNA, 25 bp, Artificial Sequence
  Description of Artificial Sequence Note = Synthetic Construct
tggatgggcggcaacatgtctgtgg

SEQ ID NO: 18 - DNA, 25 bp, Artificial Sequence
  Description of Artificial Sequence Note = Synthetic Construct
gtgaggggggcttgaccaggacgca

SEQ ID NO: 19 - DNA, 25 bp, Artificial Sequence
  Description of Artificial Sequence Note = Synthetic Construct
gttatcagtaagggagctgcagtgg

SEQ ID NO: 20 - DNA, 23 bp, Artificial Sequence
  Description of Artificial Sequence Note = Synthetic Construct
aagaccgcgaagagtttgtcctc

SEQ ID NO: 21 - DNA, 24 bp, Artificial Sequence
  Description of Artificial Sequence Note = Synthetic Construct
ggcggatcacaagcaataataacc

SEQ ID NO: 22 - DNA, 22 bp, Artificial Sequence
  Description of Artificial Sequence Note = Synthetic Construct
ggatttccgtctctggtgtagc

SEQ ID NO: 23 - DNA, 22 bp, Artificial Sequence
  Description of Artificial Sequence Note = Synthetic Construct
accattgcccctgttcactatc

SEQ ID NO: 24 - DNA, 22 bp, Artificial Sequence
  Description of Artificial Sequence Note = Synthetic Construct
accctccagctccagacttatc

SEQ ID NO: 25 - DNA, 22 bp, Artificial Sequence
  Description of Artificial Sequence Note = Synthetic Construct
ccctgtaatggattccaagctg

SEQ ID NO: 26 - DNA, 20 bp, Artificial Sequence
  Description of Artificial Sequence Note = Synthetic Construct
aaagacccctaggaatgctc